(19) United States
(12) Patent Application Publication
(10) Pub. No.: US 2024/0423797 A1
(43) Pub. Date: Dec. 26, 2024

(54) METHODS AND APPARATUS FOR REMOVAL OF VALVE REPAIR DEVICES

(71) Applicant: EDWARDS LIFESCIENCES CORPORATION, Irvine, CA (US)

(72) Inventors: Eric Michael Oberwise, Newport Beach, CA (US); Sydney Jade Fletcher, Huntington Beach, CA (US); Aviv Galon, Tel Aviv (IL); Lior Harush, Herzliya (IL); Alexander H. Cooper, Costa Mesa, CA (US); Nikolai Brent Poulsen, Tustin, CA (US); Cooper Ryan Rickerson, Huntington Beach, CA (US); Hiroshi Okabe, Tustin, CA (US); Sam Shafigh, Irvine, CA (US); Travis Zenyo Oba, Yorba Linda, CA (US)

(21) Appl. No.: 18/821,354

(22) Filed: Aug. 30, 2024

Related U.S. Application Data
(63) Continuation of application No. PCT/US2023/018018, filed on Apr. 10, 2023.
(60) Provisional application No. 63/399,360, filed on Aug. 19, 2022.
(60) Provisional application No. 63/331,762, filed on Apr. 15, 2022.

(51) Int. Cl. A61F 2/24; A61B 17/00; A61B 17/3205; A61B 18/00; A61B 18/14; A61B 18/18

(52) U.S. Cl. CPC A61F 2/2436; A61B 17/32056; A61B 18/1492; A61B 18/18; A61F 2/2418; A61B 2017/00783; A61B 2018/00369; A61B 2018/00577; A61B 2018/00595; A61B 2018/00601; A61B 2018/141; A61B 2018/1807

(57) ABSTRACT

An implantable device or implant is configured to be positioned within a native heart valve to allow the native heart valve to form a more effective seal. The implantable device can be removed using one or more of a cutting device, a capture device, and a stabilization component. The stabilization component can control the position of the implantable device while the cutting device resects one or more of the leaflets to which the implantable device is engaged.

### RELATED APPLICATIONS

The present application is a continuation of Patent Cooperation Treaty application no. PCT/US2023/018018, filed on Apr. 10, 2023, which claims the benefit of U.S. Provisional Patent Application No. 63/399,360, filed on Aug. 19, 2022, and the benefit of U.S. Provisional Patent Application No. 63/331,762, filed on Apr. 15, 2022, which are all incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While sometimes described as an implantable device for illustration purposes in various examples herein, similar configurations can be used on other devices, e.g., valve repair devices, etc., that are not necessarily implanted and may be removed after treatment.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

Certain conditions or circumstances may necessitate the removal of the implantable device from the native heart valve. In some implementations, the implantable device can be removed using a retrieval catheter through which a cutting device and/or an optional stabilization component are delivered to the device. The optional stabilization component can control the position of the implantable device while the cutting device resects one or more of the leaflets to which the implantable device is engaged. In some implementations, the stabilization component retracts the implantable device into the retrieval catheter. In some implementations, the same element functions as the stabilization method and the cutting device. In some implementations, the retrieval catheter can also optionally deploy an indicator and/or gauge to guide the cutting device.

In some implementations, a device for resecting a native leaflet includes a catheter, a cutting device, and a stabilization component. The cutting device is disposed within the catheter. The cutting device comprises a snare capable of severing or ablating the native leaflet. The stabilization component comprises an element for grasping an implantable device.

In some implementations, the cutting device can be formed of electrodes that can be comprised of a metallic element allowing current to flow. The cutting device can be made of nitinol. The cutting device can comprise a surface which allows for radiofrequency energy to ablate the native leaflet. The cutting device can serve as the stabilization component. The element for grasping the implantable device can be a snare. The element for grasping the implantable device can be a pincer, a grasper, or a vacuum suction device. A second cutting device can comprise a second snare capable of severing or ablating the native leaflet.

In some implementations, a device for resecting a native leaflet includes a catheter, a cutting device, and a stabilization component. The cutting device comprises at least one coring element disposed proximate to the catheter. The at least one coring element comprises a feature capable of severing or ablating the native leaflet. The stabilization component has an element for grasping an implantable device.

In some implementations, the at least one coring element is disposed along an external surface of the catheter. A single coring element can surround an external surface of the catheter. The device comprises a first coring element and a second coring element. The at least one coring element can be arc shaped. The feature capable of severing or ablating the native leaflet can be a blade, can be a cutting tip formed of electrodes that can be comprised of a metallic element allowing current to flow, and/or can be a surface which conducts radiofrequency energy. The at least one coring element can be made of nitinol. The element for grasping the implantable device can be a snare, a pincer, a grasper, or a vacuum suction device.

In some implementations, a device for resecting a native leaflet includes a catheter, a cutting device, and an indicator or gauge. The cutting device includes at least one electrosurgical element. The stabilization component is configured to grasp an implantable device.

In some implementations, the cutting device can be made of nitinol. The electrosurgical element can be a cutting tip or blade or can be a ring. The element for grasping the implantable device can be a snare, a pincer, a grasper, or a vacuum suction device. The device can include a second cutting device. The indicator or gauge can comprise a radiopaque feature. The indicator or gauge can be a depth gauge. The indicator or gauge can be a long, compliant positioning wire or rod. The indicator or gauge can be configured to lead the cutting device and engage tissue to be cut before the cutting device.

In some implementations, a method of resecting a native leaflet includes deploying a catheter to an implantable device secured to at least one leaflet of a native heart valve. A cutting device is deployed from the catheter to the at least one leaflet. At least one of the cutting device and a stabilization component are secured to a portion of the implantable device. The native leaflet is resected with the cutting device. A second native leaflet is resected with the cutting device. The implantable device is removed from the native heart valve using at least one of the cutting device and the stabilization component via the catheter.

The above method(s) can be performed on a living animal or on a simulation, such as on a cadaver, cadaver heart, anthropomorphic ghost, simulator (e.g., with the body parts, heart, tissue, etc. being simulated), etc.

In some implementations, a device for resecting a native leaflet includes a catheter and a clamp. The clamp includes a first grasping arm and a second grasping arm. The clamp includes an element configured for cutting, severing, or resecting the native leaflet.

In some implementations, upon closure of the first and second grasping arms of the clamp, the first and second grasping arms form a complete encapsulation with a void in a center thereof to secure an implantable device. The first and second grasping arms can each include serrated edges or blades and/or an electrocautery element.

In some implementations, a device for resecting a native leaflet includes a catheter, a cutting device, and a stabilization component. The cutting device includes a central wire, a first prong, and a second prong. The first and second prongs are configured to cut, sever, and/or ablate the native leaflet. The stabilization component is configured to grasp an implantable device.

In some implementations, the first and second prongs comprise sharp blades and/or an electrocautery element. The cutting device can be connected to an infrared generator, such that heat can be used to sever the native leaflet. The first and second prongs can comprise a surface that allows radiofrequency energy to ablate the native leaflet. The cutting device can be made of nitinol. The device can comprise a comprising a balloon. The balloon can include cutting structures capable of cutting, severing and/or ablating the native leaflet such as through the use of electrocautery, vibration, blades, or heat.

In some implementations, a device for resecting a native leaflet can include a hook and a loop that are configured to be joined around the native leaflet and to cut the native leaflet.

In some implementations, the hook can extend from a first catheter and the loop can extend from a second catheter. The device can include a stabilization component configured to grasp an implantable device. The hook and the loop can be capable of cutting, severing and/or ablating the native leaflet, such as through the use of friction, heat, electrocautery, vibration, blades, and/or serration. The hook and the loop can be formed of electrodes that can be comprised of a metallic element allowing current to flow. The hook and the loop can be connected to an infrared generator, such that heat can be used to sever the native leaflet. The hook and the loop can be made of nitinol or spring wire. The loop can align perpendicularly to the loop when it deploys. The hook can be aligned to enter the loop creating a lasso that can be used to sever the native leaflet.

In some implementations, a device for resecting a native leaflet includes a catheter and a retrieval device. The retrieval device can include a positioning element (e.g., a wire, line, etc.), a snare, and/or a bag. The snare is capable of cutting, severing, and/or ablating the native leaflet such as through the use of friction, heat, electrocautery, vibration, blades, serration. In some implementations, the bag is connected to the snare and is configured to contain and retrieve a valve repair device and severed leaflet portions.

In some implementations, the retrieval device (e.g., snare, etc.) can be comprised of an electrocautery element. The retrieval device (e.g., snare, etc.) can be connected to an infrared generator, such that the heat can be used to sever the native leaflet. The retrieval device, snare, and/or the bag can be made of nitinol or spring wire to allow for shape-memory characteristics. The retrieval device (e.g., snare, etc.) can be comprised of surfaces which allow for radiofrequency energy to ablate the native leaflet.

In some implementations, a device for resecting a native leaflet includes a catheter, a cap, a cutting element, and/or an actuation element. The cap is attached to the catheter such that the cap is movable between an open position and a closed position relative to the catheter. The actuation element can be configured to move the cap between the open and closed positions. The cutting element is capable of cutting, severing, or ablating the native leaflet. The cutting element can be attached to the cap and/or the catheter. The cap is configured to capture a valve repair device.

In some implementations, the cutting element can be comprised of an electrocautery element. The cutting element can be connected to an infrared generator, such that the heat can be used to sever the native leaflet. The cutting element and/or the cap can be made of nitinol or spring wire to allow for shape-memory characteristics. The cutting element can be comprised of surfaces which allow for radiofrequency energy to ablate the native leaflet.

In some implementations, the cap is biased in a closed position, and the actuation element is used to move the cap from the closed position to the open position. In some implementations, the cap is biased in an open position, and the actuation element is used to move the cap from the open position to the closed position.

In some implementations a method for resecting one or more native leaflets of a native valve and implanting a replacement valve in the native valve includes deploying a cutting device such that the cutting device is positioned proximate the native valve. The method can further include cutting one or more native leaflets with the cutting device such that a valve repair device is removed from the one or more native leaflets, where the valve repair device remains connected to at least one other native leaflet of the native valve. The method can further include deploying a replacement valve such that the replacement valve is positioned proximate the native valve, where the replacement valve has a body and one or more anchors. The method can further include attaching the replacement valve to at least a portion of the one or more native leaflets and the at least one other native leaflet such that the valve repair device is captured by the replacement valve between the body and the one or more anchors.

In some implementations, the one or more native leaflets are engaged with the cutting device prior to cutting the one or more native leaflets, and the cutting device is repositioned relative to the one or more native leaflets if tenting of the one or more native leaflets is not detected.

In some implementations, cutting comprises the use of at least one of friction, electrocautery, vibration, and serration.

In some implementations, the cutting device comprises one or more blades.

In some implementations, the cutting device comprises an electrosurgical tip formed of electrodes that includes a metallic element configured to allow current to flow.

In some implementations, the cutting device comprises an electrosurgical tip formed of electrodes that includes a metallic element configured to allow current to flow.

In some implementations, the cutting device is connected to an infrared generator such that heat can be used to sever the one or more native leaflets.

In some implementations, the cutting device comprises one or more surfaces that allow for radiofrequency energy to ablate the one or more native leaflets.

In some implementations, a delivery system is positioned proximate the native valve and the delivery system is configured to deploy the cutting device and the replacement valve.

In some implementations, a stabilization component connects the valve repair device to the delivery system prior to cutting the one or more native leaflets.

In some implementations, the cutting device is deployed from a first catheter of the delivery system and the replacement valve is deployed from a second catheter of the delivery system.

In some implementations, the body comprises an inner body or frame and an outer body or frame.

In some implementations, a system for resecting a native leaflet includes a catheter having a first lumen and a second lumen, a first cutter delivery catheter configured to be delivered through the first lumen, a second cutter delivery catheter configured to be delivered through the second lumen, and a cutting element configured to extend through the third lumen.

In some implementations, a first distal tip of the first cutter delivery catheter includes a first coupling element configured to connect to a second coupling element on a second distal tip of the second cutter delivery catheter such that the cutting element is advanceable through the second lumen.

In some implementations, the first coupling element is configured to magnetically couple to the second coupling element. In some implementations, the second cutter delivery catheter includes a fourth lumen and, when the first distal tip is connected to the second distal tip, the third lumen is aligned with the fourth lumen. In some implementations, the cutting element is advanceable through the second lumen via the fourth lumen.

In some implementations, the first coupling element and/or the second coupling element is a magnet. In some implementations, the first coupling element is a first annular magnet, and the second coupling element is a second annular magnet.

In some implementations, the first coupling element is configured to mechanically couple to the second coupling element. In some implementations, the first coupling element is configured as a female connector and the second coupling element is configured as a male connector. In some implementations, the first coupling element is configured to be received in the third lumen at the first distal tip. In some implementations, the first coupling element has a first exterior surface having a complementary shape to an interior surface of the first distal tip.

In some implementations, the first coupling element includes a distal end and a proximal end, wherein the cutting element is attached to the proximal end. In some implementations, the distal end is configured to receive the second coupling element. In some implementations, the second coupling element includes one or more radially outward extending projections.

In some implementations, the first coupling element includes one or more radially inward extending projections configured to engage the one or more radially outward extending projections to resist separation of the second coupling element from the first coupling element. In some implementations, the first coupling element is configured to be removed from the first cutter delivery catheter once coupled to the second coupling element.

In some implementations, the cutting element is a conductive wire. In some implementations, an activation source is configured to energize the cutting element. In some implementations, the activation source is a radio-frequency generator.

In some implementations, the first cutter delivery catheter has a distal end portion that is steerable. In some implementations, the first cutter delivery catheter has a distal end portion that has shape-memory characteristics.

In some implementations, an inflatable balloon is attached to an exterior surface of the first cutter delivery catheter adjacent the first distal tip. In some implementations, an inflatable balloon is attached to an exterior surface of the second cutter delivery catheter adjacent the second distal tip.

In some implementations, a system for resecting a native leaflet includes a delivery catheter and a cutting device configured for delivery through the delivery catheter. In some implementations, the cutting element includes a first arm having a first distal end, a second arm having a second distal end spaced apart from the first distal end, and a cutting element extending between the first distal end and the second distal end.

In some implementations, the cutting element is a conductive wire. In some implementations, the conductive wire is in a slack condition between the first distal end and the second distal end. In some implementations, the first arm and the second arm form a V-shape.

In some implementations, the first arm and the second arm are insulated conductive wire and the cutting element is uninsulated conductive wire. In some implementations, an activation source is configured to energize the cutting element. In some implementations, the activation source is a radio-frequency generator.

In some implementations, a method of resecting a native valve leaflet captured by an implantable device includes extending a first cutter delivery catheter from an atrial side of the native valve leaflet to a ventricular side on a first side of the implantable device, extending a second cutter delivery catheter from the atrial side of the native valve leaflet to the ventricular side on a second side of the implantable device opposite the first side, and/or connecting the first cutter delivery catheter to the second cutter delivery catheter on the ventricular side of the native valve leaflet.

In some implementations, the method further includes extending a cutting element through a first lumen in the first cutter delivery catheter and a second lumen in the second cutter delivery catheter, withdrawing the first cutter delivery catheter to expose the cutting element adjacent the native valve leaflet, and/or moving cutting element through the native valve leaflet from the ventricular side to the atrial side to detach the native valve leaflet from the implantable device.

In some implementations, connecting the first cutter delivery catheter to the second cutter delivery catheter further includes magnetically coupling the first cutter delivery catheter to the second cutter delivery catheter.

In some implementations, connecting the first cutter delivery catheter to the second cutter delivery catheter further includes aligning the first lumen in the first cutter delivery catheter with the second lumen in the second cutter delivery catheter. In some implementations, the method includes steering a first distal tip of the first cutter delivery catheter toward a second distal tip of the second cutter delivery catheter in the ventricular side.

In some implementations, the method includes connecting the cutting element to an activation source. In some implementations, the activation source is a radio frequency generator. In some implementations, the method includes activating the cutting element with radiofrequency energy. In some implementations, the cutting element is a conductive wire.

In some implementations, extending the first cutter delivery catheter from the atrial side of the native valve leaflet to the ventricular side further includes inflating a balloon on an exterior surface of the first cutter delivery catheter.

In some implementations, extending the second cutter delivery catheter from the atrial side of the native valve leaflet to the ventricular side further includes inflating a second balloon on an exterior surface of the second cutter delivery catheter.

In some implementations, a method of resecting a native valve leaflet captured by an implantable device includes extending a first cutter delivery catheter from an atrial side of the native valve leaflet to a ventricular side on a first side of the implantable device, extending a second cutter delivery catheter from the atrial side of the native valve leaflet to the ventricular side on a second side of the implantable device opposite the first side, and connecting the first cutter delivery catheter to the second cutter delivery catheter on the ventricular side of the native valve leaflet.

In some implementations, a cutting element is connected to a first coupling element associated with the first cutter delivery catheter. In some implementations, the method further includes withdrawing the first cutter delivery catheter to expose the cutting element adjacent the native valve leaflet and moving cutting element through the native valve leaflet from the ventricular side to the atrial side to detach the native valve leaflet from the implantable device.

In some implementations, connecting the first cutter delivery catheter to the second cutter delivery catheter further includes mechanically coupling the first cutter delivery catheter to the second cutter delivery catheter. In some implementations, mechanically coupling the first cutter delivery catheter to the second cutter delivery catheter further includes receiving a male connector into a female connector.

In some implementations, withdrawing the first cutter delivery catheter further includes detaching the female connector from the first cutter delivery catheter. In some implementations, detaching the female connector from the first cutter delivery catheter further includes applying a tensile force to one or both of the first cutter delivery catheter and the second cutter delivery catheter.

In some implementations, the method includes connecting the cutting element to an activation source. In some implementations, the activation source is a radio frequency generator. In some implementations, the method includes activating the cutting element with radiofrequency energy. In some implementations, the cutting element is a conductive wire.

In some implementations, extending the first cutter delivery catheter from the atrial side of the native valve leaflet to the ventricular side further includes inflating a balloon on an exterior surface of the first cutter delivery catheter.

In some implementations, connecting the first cutter delivery catheter to the second cutter delivery catheter on the ventricular side of the native valve leaflet further includes steering a distal end portion of the first cutter delivery catheter toward the second cutter delivery catheter.

In some implementations, a method of resecting a native valve leaflet captured by an implantable device includes delivering a distal tip of a delivery catheter to an atrial side of the native valve leaflet, supporting a cutting element adjacent the atrial side of the native valve leaflet, and moving cutting element through the native valve leaflet from the atrial side to a ventricular side to detach the native valve leaflet from the implantable device. In some implementations, the cutting element is supported adjacent the atrial side of the native valve leaflet in a slack condition.

In some implementations, the method further includes connecting the cutting element to an activation source. In some implementations, the activation source is a radio frequency generator. In some implementations, the method further includes activating the cutting element with radiofrequency energy. In some implementations, the cutting element is a conductive wire.

In some implementations, supporting the cutting element adjacent the atrial side of the native valve leaflet further includes extending the cutting element between a first distal end of a first arm and a second distal end of a second arm. In some implementations, the method further includes extending the first arm and the second arm from the distal tip of the delivery catheter.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example device or implant of FIGS. 8-14 being delivered and deployed within a native valve;

FIG. 22 shows a perspective view of an example device or implant in a closed position;

FIG. 23 shows a perspective view of an example device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of the connector of the implantable device and coupled to a paddle width control;

FIG. 31 illustrates a front view of an example implantable device in a closed position engaged with the anterior and posterior leaflets of a mitral valve;

FIG. 32 shows a front view of an example retrieval catheter, a cutting device, and an optional stabilization component;

FIG. 33 shows a side view of the example retrieval catheter, cutting device, and optional stabilization component of FIG. 32;

FIG. 34 shows a front view of the example retrieval catheter, cutting device, and optional stabilization component of FIG. 32, wherein the cutting device is partially retracted, and the retrieval catheter is proximate the anterior and posterior leaflets;

FIG. 35 shows a side view of the example retrieval catheter, cutting device, and optional stabilization component as illustrated in FIG. 34;

FIG. 36 shows a front view of an example retrieval catheter, two cutting devices, and an optional stabilization component;

FIG. 37 shows a side view of the example retrieval catheter, two cutting devices, and optional stabilization component of FIG. 36;

FIG. 38 shows a front view of the example retrieval catheter, two cutting devices, and optional stabilization component of FIG. 36, wherein the cutting device is partially retracted, and the retrieval catheter is proximate the anterior and posterior leaflets;

FIG. 39 shows a side view of the example retrieval catheter, cutting device, and optional stabilization component as illustrated in FIG. 38;

FIG. 40 illustrates an example retrieval catheter, a cutting device, and a stabilization component;

FIG. 41 shows the example retrieval catheter, cutting device, and stabilization component of FIG. 40 with the cutting device and catheter advanced to the leaflets;

FIG. 42 illustrates the example retrieval catheter, cutting device, and stabilization component of FIG. 40, wherein the cutting device is cutting through the leaflet;

FIG. 43 shows the example retrieval catheter, cutting device, and stabilization component of FIG. 40, wherein the leaflet and valve repair device are being pulled into the retrieval catheter by the stabilization component;

FIG. 44 shows a top view of an example retrieval catheter, a stabilization component, a cutting device, and an indicator or gauge. The retrieval catheter is shown off-center to allow for all features of the drawing to be visible;

FIG. 45 is a front view of the example retrieval catheter, stabilization component, cutting device, and indicator or gauge of FIG. 44;

FIG. 46 is a front view of an example retrieval catheter, a stabilization component, and two cutting devices;

FIG. 47 is a top view of the example retrieval catheter, stabilization component, and two cutting devices of FIG. 46;

FIG. 48 is a front view of an example retrieval catheter, a stabilization component, and a cutting device;

FIG. 49 is a top view of the example retrieval catheter, stabilization component, and cutting device of FIG. 48;

FIGS. 50A-50B illustrate a method of removing an example implantable device using an example cutting device;

FIGS. 51A-51C illustrate a method of removing an example implantable device using an example cutting device;

FIG. 52 is a front view of an example cutting device;

FIG. 53 shows an example retrieval catheter and the cutting device of FIG. 52;

FIGS. 54A-54C show example positions of the retrieval catheter and cutting device of FIG. 52;

FIGS. 54D-54F illustrate a method of cutting a leaflet attached to an example implantable device using the retrieval catheter and cutting device of FIG. 52;

FIGS. 55A-55B illustrate a method of retracting the example cutting device into the retrieval catheter of FIG. 52;

FIGS. 56A-56B illustrate a method of removing an example implantable device from a leaflet using the retrieval catheter and cutting device of FIG. 52;

FIGS. 57A-57B illustrate a method of retrieving an example implantable device using a first retrieval catheter, a second retrieval catheter, and two cutting devices;

FIGS. 58A-58B illustrate a method of retrieving an example implantable device using a retrieval catheter, a cutting device, and a stabilization component;

FIGS. 59A-59B illustrate a method of retracting the cutting device and stabilization component of FIGS. 58A-58B into the retrieval catheter;

FIGS. 60A-60B illustrate an example retrieval catheter and a cutting device;

FIGS. 61A-61C illustrate a method of retrieving an example implantable device using the retrieval catheter and the cutting device of FIGS. 60A-60B;

FIGS. 62A-62H illustrate a method of removing an example implantable device from at least one leaflet of a native heart valve using an example cutting device;

FIGS. 63A-63D illustrate a method of implanting a replacement valve onto a native valve such that the replacement valve captures an implantable device that is attached to the native valve;

FIG. 64 illustrates an example replacement valve;

FIGS. 65A-65E illustrate a method of implanting the replacement valve of FIG. 64 onto a native valve such that the replacement valve captures an implantable device that is attached to the native valve;

FIGS. 66-71 illustrate an example cutting device for detaching an implantable device from a valve leaflet;

FIG. 72 illustrates an example cutting device for detaching an implantable device from a valve leaflet;

FIGS. 73-77 illustrate an example cutting device for detaching an implantable device from a valve leaflet;

FIGS. 78-83 illustrate an example cutting device for detaching an implantable device from a valve leaflet; and

FIG. 84 illustrates an example cutting device for detaching an implantable device from a valve leaflet.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

The treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

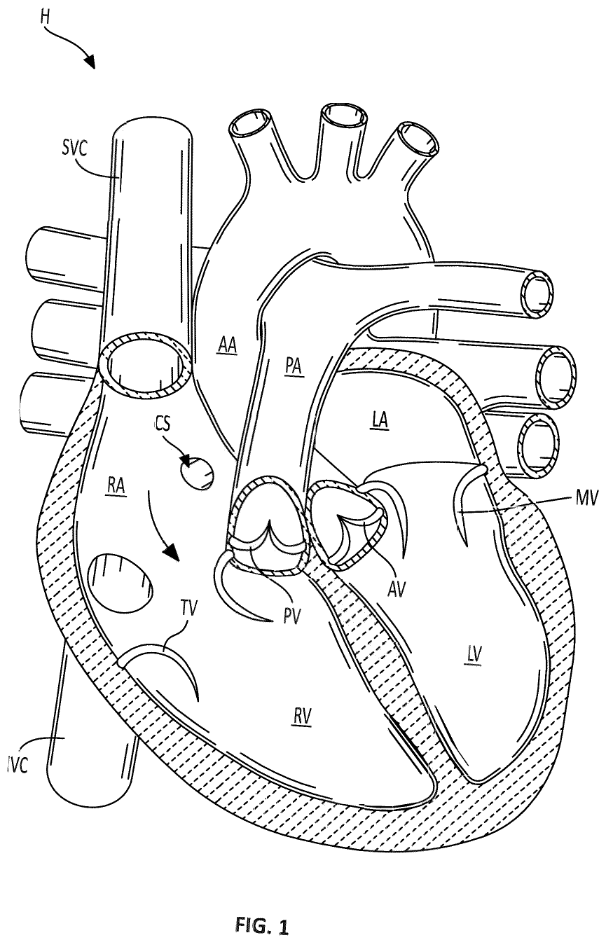

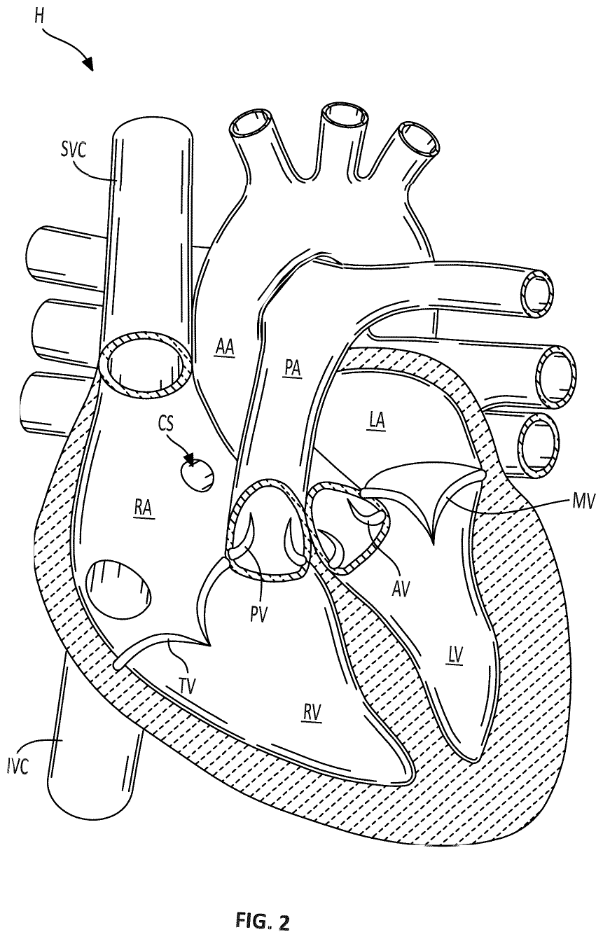

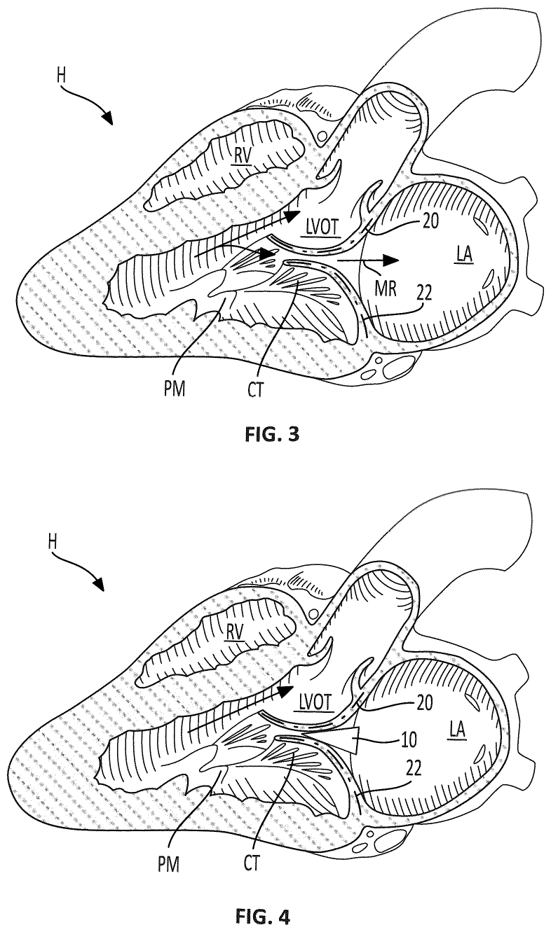

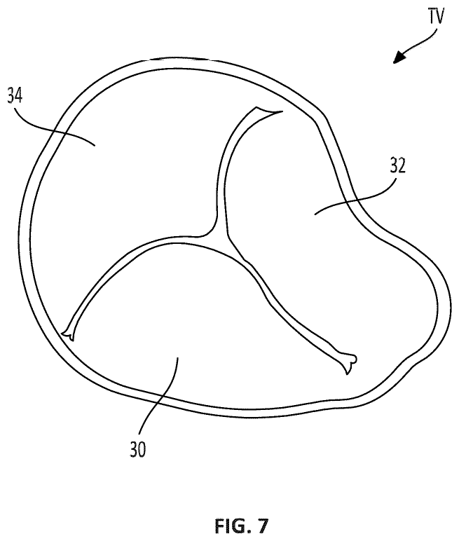

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

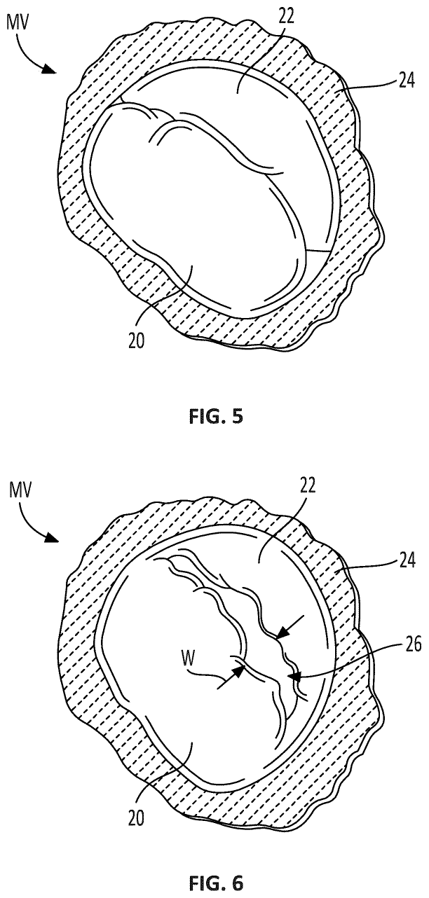

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, an implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example device or implant can optionally have a coaptation element (e.g., spacer, coaption element, gap filler, membrane, sheet, plug, wedge, balloon, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaption element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, anthropomorphic ghost, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

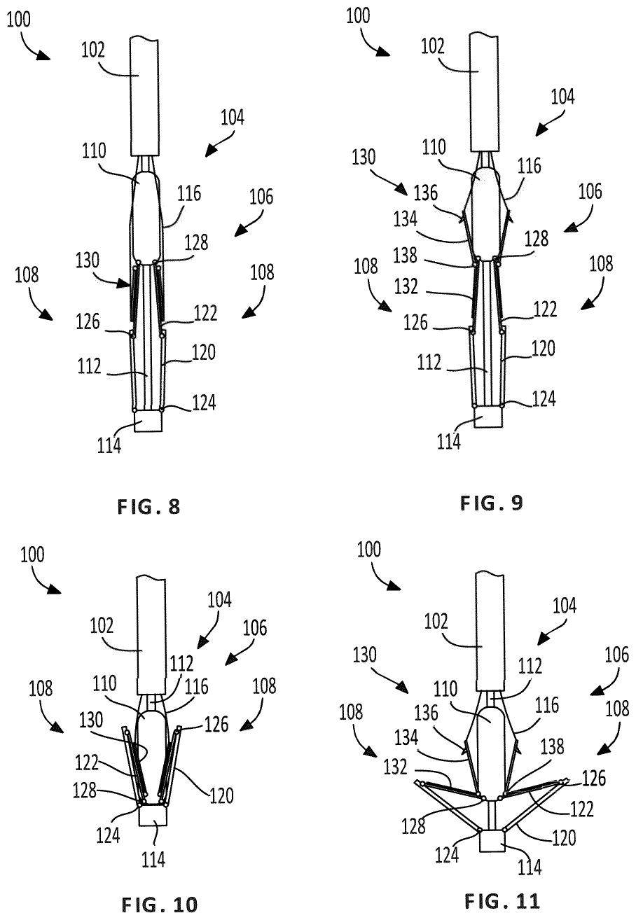

Referring now to FIGS. 8-15, a schematically illustrated device or implant 100 (e.g., a prosthetic device, a valve repair device, implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

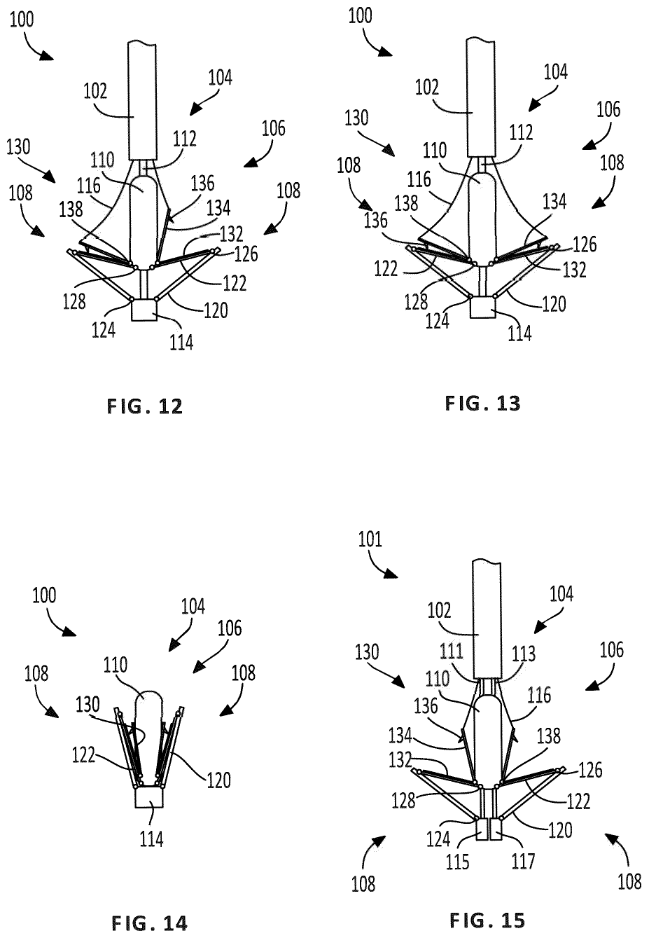

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

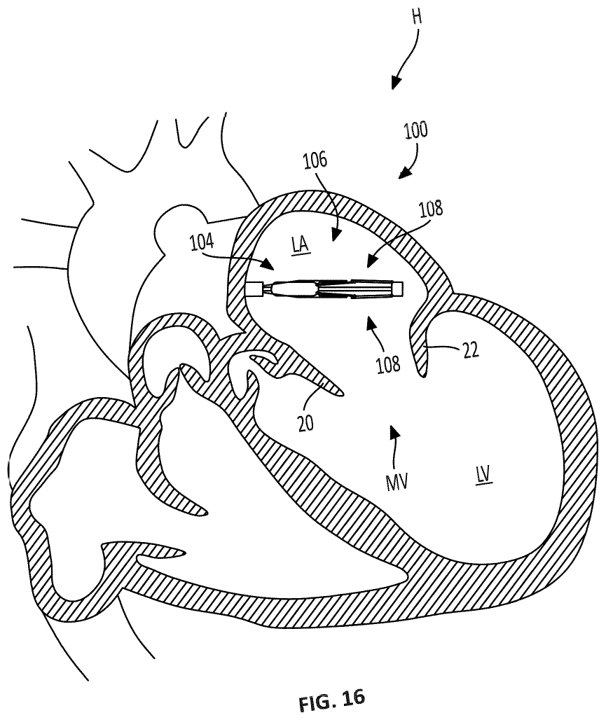

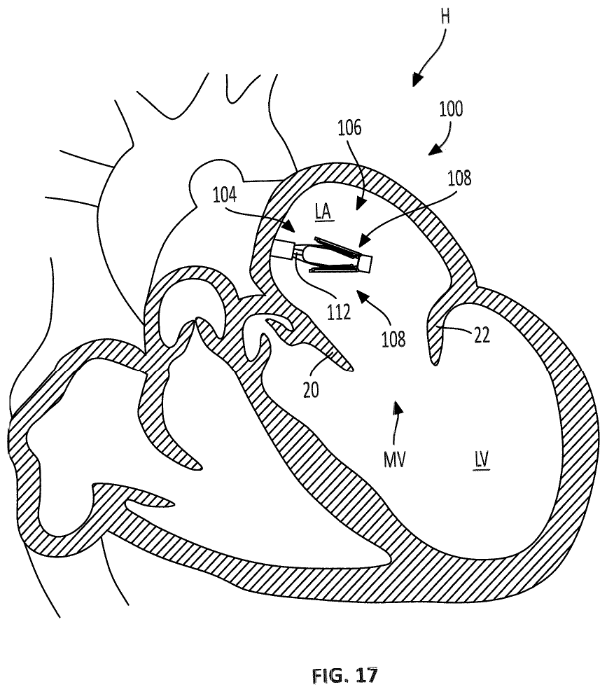

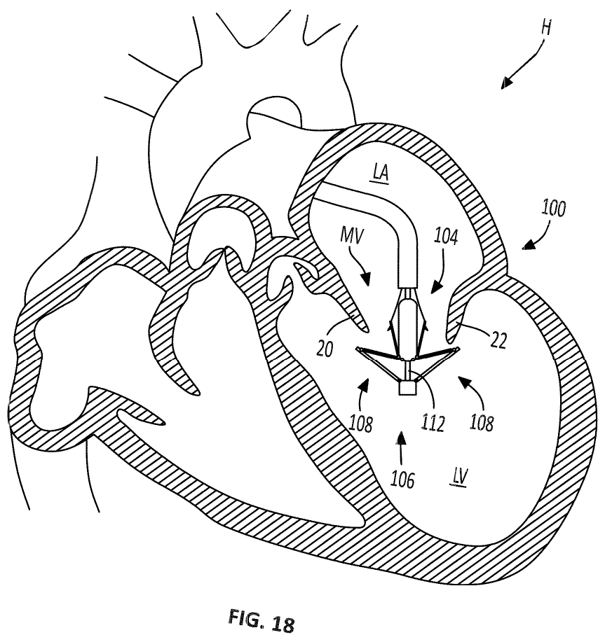

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

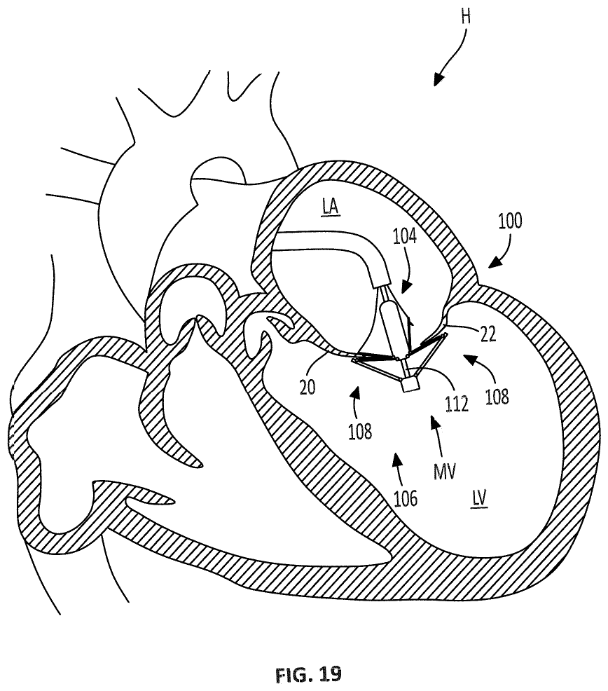

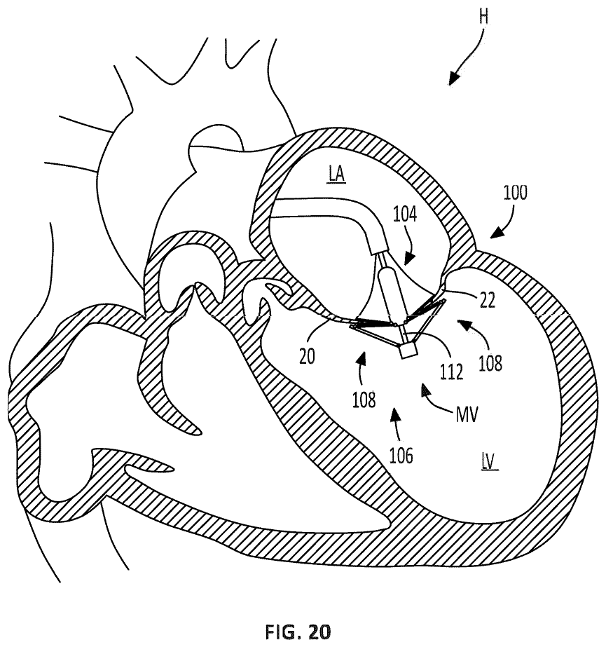

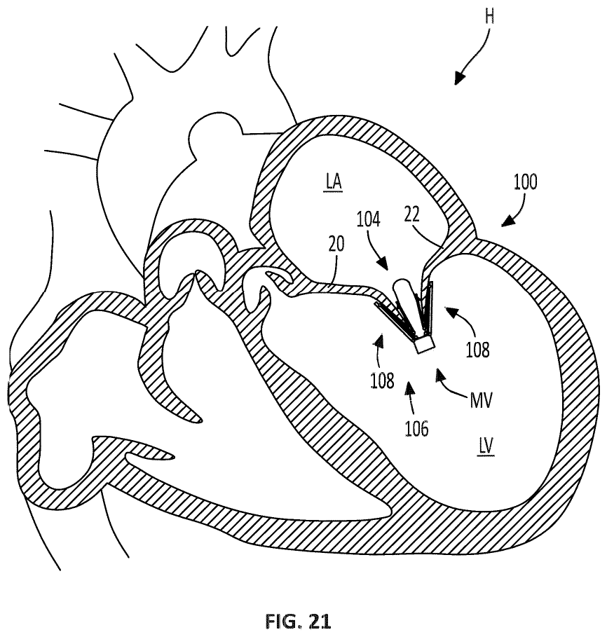

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

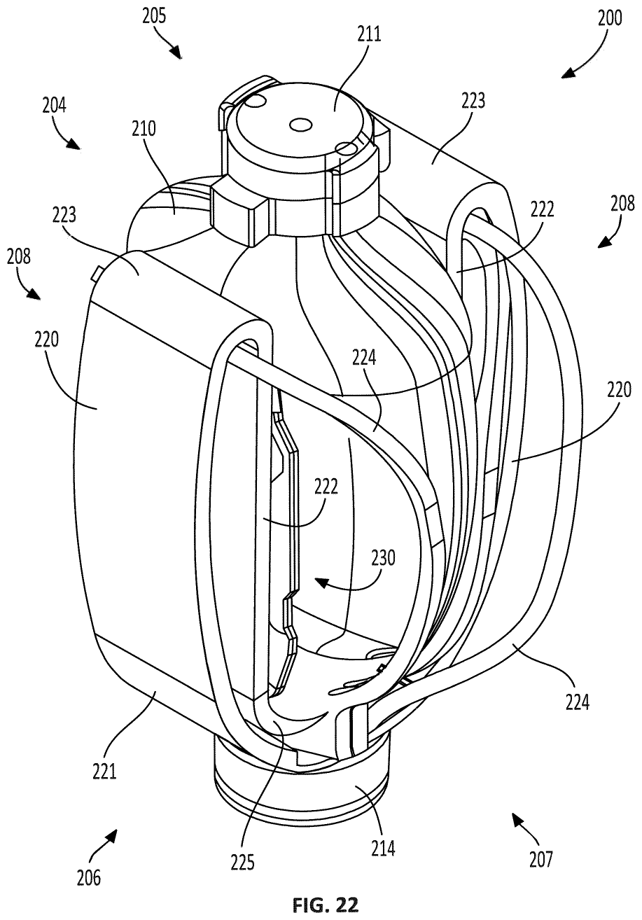

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., spacer, coaption element, gap filler, membrane, sheet, plug, wedge, balloon, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

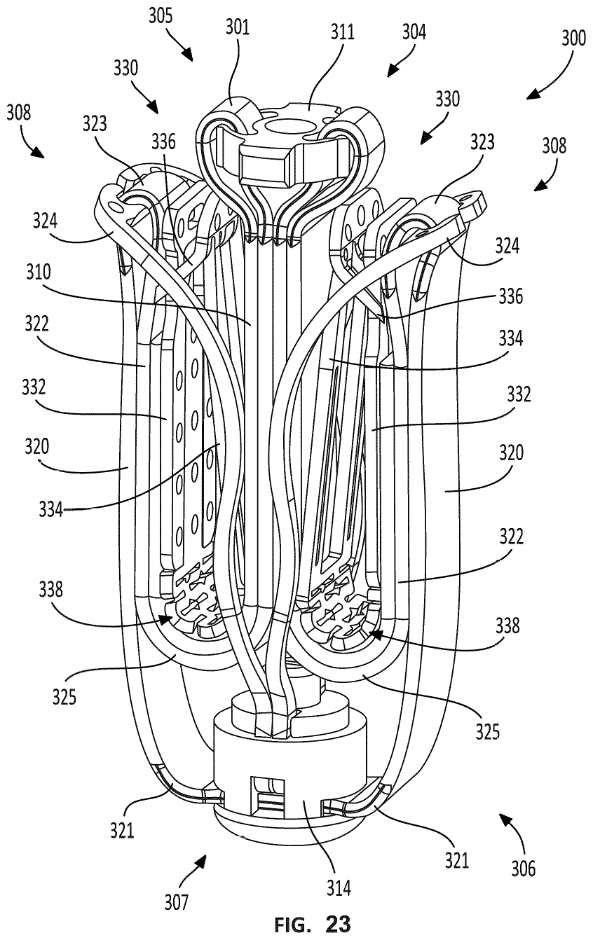

Referring now to FIG. 23, an example of a device or implant 300 is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable valve repair system (e.g., any valve repair system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose optional the optional barbs 336. The clasps 330 are opened by applying tension to actuation lines attached to the moveable arms 334, thereby causing the moveable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

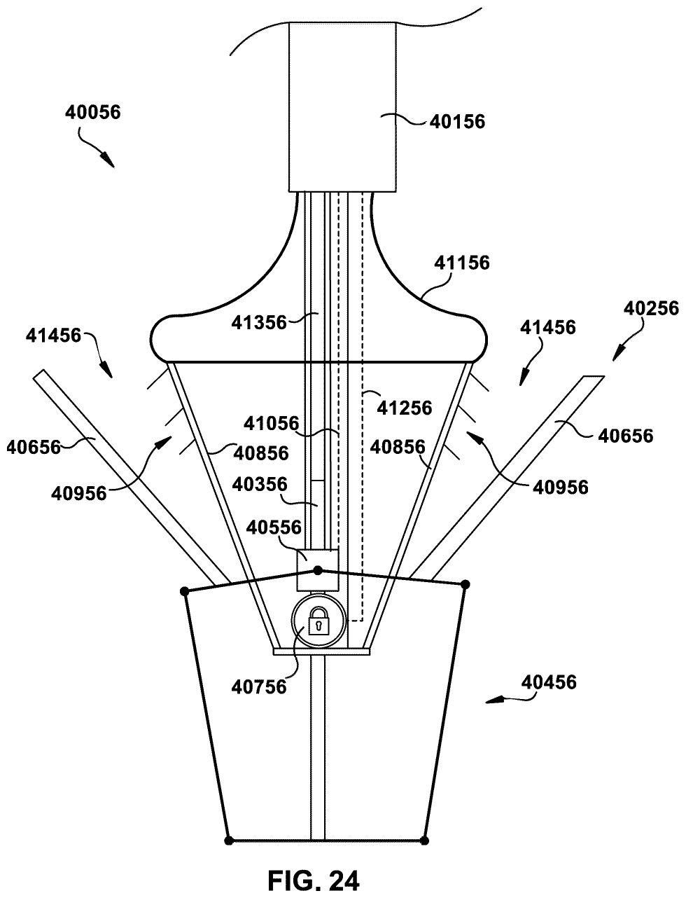

FIG. 24 illustrates an example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856 (e.g., clasps, clasp arms, grippers, gripping arms, latches, etc.). In one example, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include an optional barbed portion 40956 for attaching the gripping members to valve tissue when the valve repair device 40256 is attached to the valve tissue. When the paddles 40656 are in the closed position, the paddles engage the gripping members 40856, such that, when valve tissue is attached to the barbed portion 40956 of the gripping members, the paddles secure the valve repair device 40256 to the valve tissue. In some implementations, the gripping members 40856 are configured to engage the paddles 40656 such that the optional barbed portion 40956 engages the valve tissue member and the paddles 40656 to secure the valve repair device 40256 to the valve tissue member. For example, in certain situations, it can be advantageous to have the paddles 40656 maintain an open position and have the gripping members 40856 move outward toward the paddles 40656 to engage valve tissue and the paddles 40656.

While the example shown in FIG. 24 illustrates a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056, a gripper control mechanism 41156, and a lock control mechanism 41256. The paddle control mechanism 41056 is mechanically attached to the coupler 40556 to move the coupler along the shaft, which causes the paddles 40656 to move between the open and closed positions. The paddle control mechanism 41056 can take any suitable form and can comprise, for example, a shaft, wire, tube, hypotube, rod, suture, line, etc. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture, a wire, a rod, a catheter, a tube, a hypotube, etc.

The lock control mechanism 41256 is configured to lock and unlock the lock. The lock 40756 locks the coupler 40556 in a stationary position with respect to the shaft 40356 and can take a wide variety of different forms and the type of lock control mechanism 41256 can be dictated by the type of lock used. In examples in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position.

The valve repair device 40256 is movable from an open position to a closed position. The base assembly 40456 includes links that are moved by the coupler 40556. The coupler 40556 is movably attached to the shaft 40356. In order to move the valve repair device from the open position to the closed position, the coupler 40556 is moved along the shaft 40356, which moves the links.

The gripper control mechanism 41156 is moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripping members 40856. When the line(s) is pulled, the gripping members 40856 move inward, which causes the opening 41456 between the gripping members and the paddles 40656 to become wider.

In order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

After the paddles 40656 are moved to the closed position, the lock 40756 is moved to the locked condition by the locking control mechanism 41256 to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 is disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

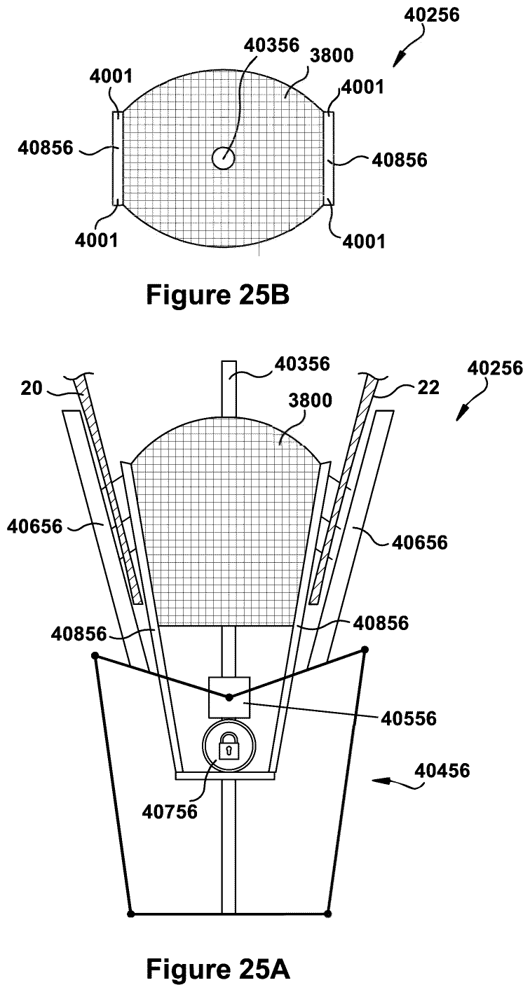

Referring to FIGS. 25A-25B, an example implementation of a valve repair device 40256 has a coaptation element 3800. The valve repair device 40256 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 40656 to adjust the size of the coaptation element. In the example illustrated by FIGS. 25A and 25B, the size of the coaptation element 3800 can be reduced by squeezing the coaptation element with the paddles 40656 and can be increased by moving the paddles 40656 away from one another. The coaptation element 3800 can extend past outer edges 4001 of the gripping members or clasps 40856 as illustrated for providing additional surface area for closing the gap of a mitral valve.

The coaptation element 3800 can be coupled to the valve repair device 40256 in a variety of different ways. For example, the coaptation element 3800 can be fixed to the shaft 40356, can be slidably disposed around the shaft, can be connected to the coupler 40556, can be connected to the lock 40755, and/or can be connected to a central portion of the clasps or gripping members 40856. In some implementations, the coupler 40656 405 can take the form of the coaptation element 3800. That is, a single element can be used as the coupler 40556 that causes the paddles 40656 to move between the open and closed positions and the coaptation element 3800 that closes the gap between the leaflets 20, 22 when the valve repair device 40256 is attached to the leaflets.

The coaptation element 3800 can be disposed around one or more of the shafts or other control elements of the valve repair system 40056. For example, the coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

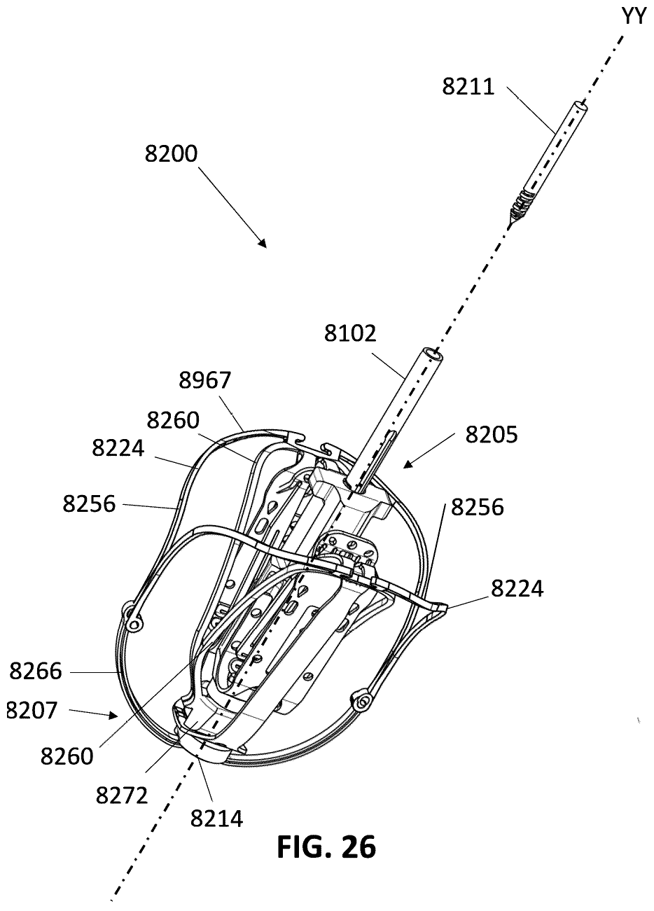

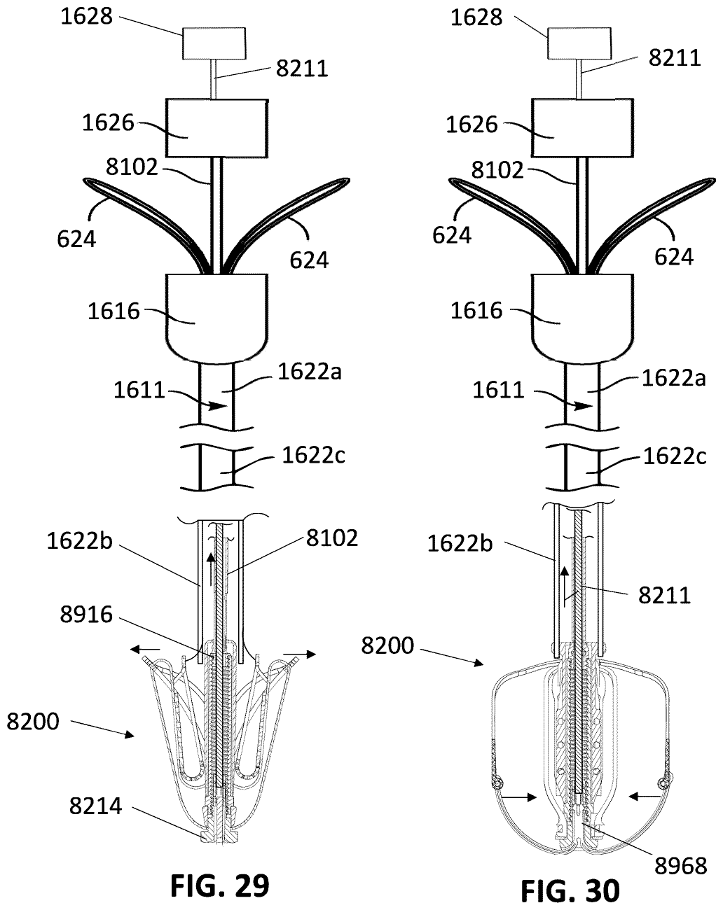

FIGS. 26-30 illustrate an example of one of the many valve repair systems for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY However, in some implementations, the paddle frames 8224 are not symmetric about the axis YY Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

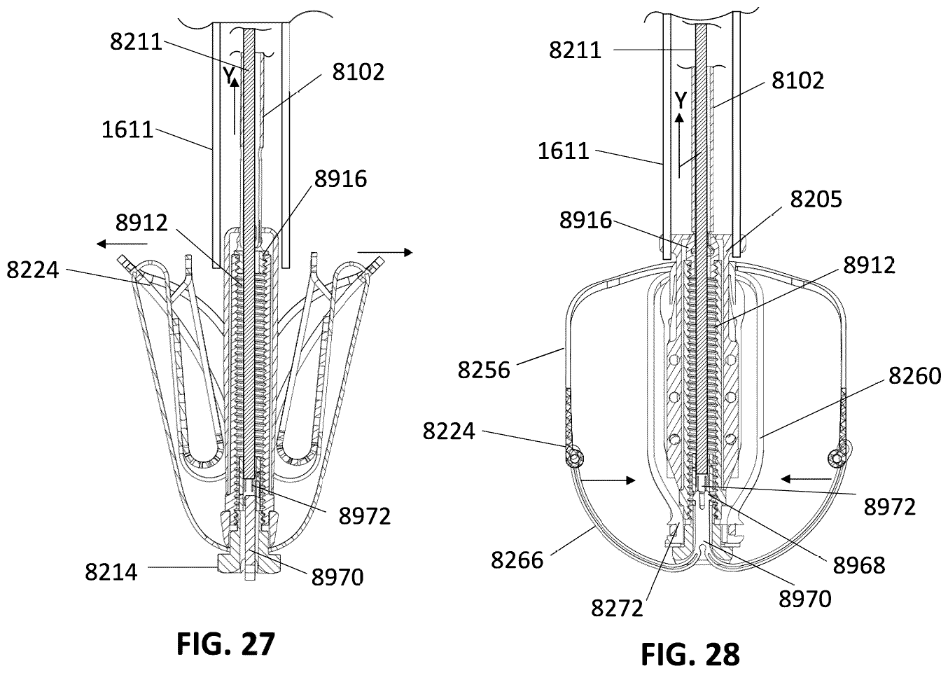

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as the other implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

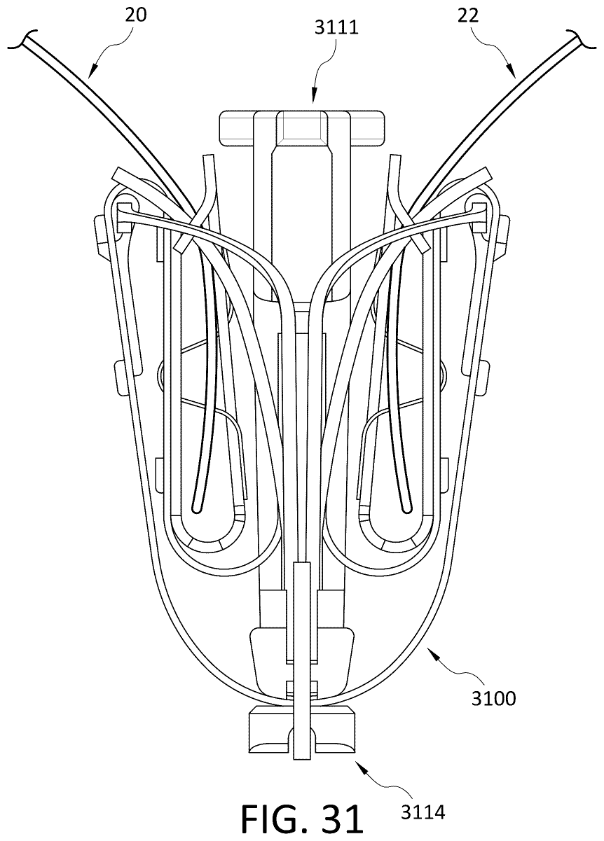

Turning now to FIGS. 31-59B, various devices and methods employed to retrieve an implantable device from the native heart valve are shown. On occasion it can be necessary to resect the native leaflets (e.g., mitral leaflets 20, 22 or tricuspid leaflets 30, 32, 34) and remove the implantable device from a patient's body. Existing solutions for the removal of the implantable device often require surgical intervention. Therefore, it is advantageous to utilize minimally invasive, transcatheter-based methods for the retrieval of an implantable device. The devices and methods described below, as shown in FIGS. 31-59B can be employed to retrieve any of the implantable devices described herein from a patient's body or any other type of implantable valve repair device. Although the anterior and posterior leaflets 20, 22 of the mitral valve are shown and described in the example implementations below, use of the devices and methods of FIGS. 31-59B is not limited to the mitral leaflets 20, 22, and can be employed on any of the native heart valves described herein. In each of the implementations described below, the various devices can be deployed using a transfemoral, transapical, or transaortic approach, such as from either the atrial side or ventricle side of the patient's heart. Further, these method(s) can be performed on a living animal or on a simulation, such as on a cadaver, cadaver heart, anthropomorphic ghost, simulator (e.g., with the body parts, heart, tissue, etc. being simulated), etc.

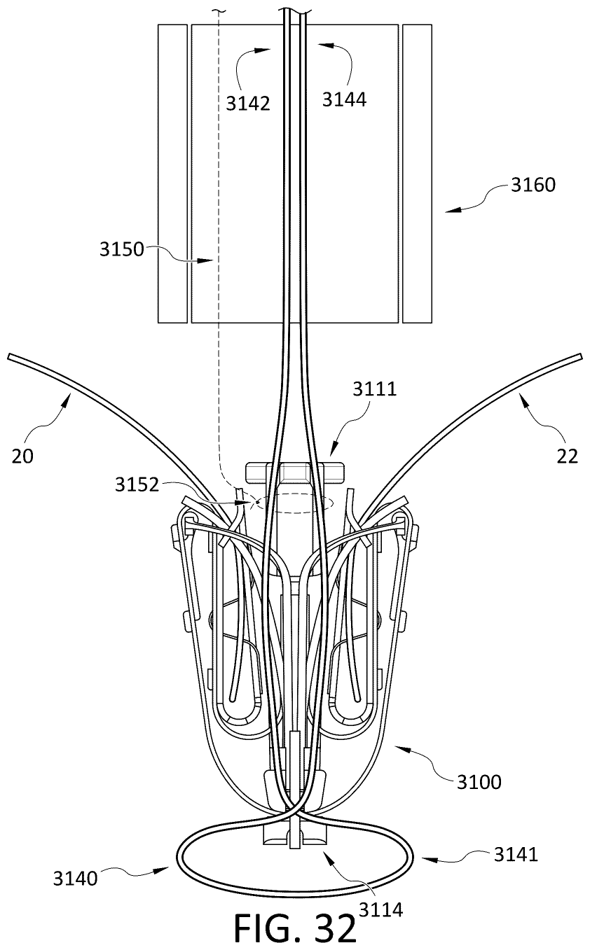

FIGS. 31-39 illustrate example devices and methods for retrieving an implantable device 3100 using at least one cutting device 3140 (See FIG. 32), wherein the cutting device 3140 comprises a snare or lasso 3141. FIG. 31 shows a front view of an implantable device 3100 attached to native leaflets (e.g., mitral valve anterior and posterior leaflets 20, 22, as shown). In some implementations, the implantable device 3100 has a collar 3111 at the proximal end and a cap 3114 at the distal end of the implantable device 3100. FIG. 32 shows a front view of an example retrieval catheter 3160, a cutting device 3140, and an optional stabilization component 3150.

The example retrieval catheter 3160 provides the conduit through which the cutting device 3140 and stabilization component 3150 are delivered to the implantable device 3100. In some implementations, the implantable device 3100 is removed from the native heart valve via retrieval catheter 3160. In some implementations, the distal end of the retrieval catheter 3160 can also be used to provide a downward force on the proximal side of the anterior and posterior leaflets 20, 22 and/or the implantable device 3100 in order to keep the anterior and posterior leaflets 20, 22 taut and/or the implantable device 3100 stable during the resecting process. In some implementations, the stabilization component 3150 and the cutting device 3140 are each deployed via separate catheters or the stabilization component 3150 and the cutting device 3140 can be deployed by a single catheter as illustrated by FIG. 32. The inside diameter of the retrieval catheter 3160 can have an increased inner diameter such that an implantable device 3100 and portions of the dissected anterior and posterior leaflets 20, 22 can be retracted therethrough.

In some implementations, the cutting device 3140 comprises a snare or lasso 3141. The cutting device 3140 can have a first end 3142 and a second end 3144, wherein at least one end, and in some implementations, both ends 3142, 3144, extend upwards through the distal end of the retrieval catheter 3160 and are secured to a retrieval component or device at the proximal end of the retrieval catheter 3160, such that the user can extend or retract the cutting device 3140 within the retrieval catheter 3160. In some implementations, the snare or lasso 3141 is equipped with a feature for cutting, severing, or ablating the anterior and posterior leaflets 20, 22, such as through the use of friction, electrocautery, vibration, serration, a sharp edge, or the like. In some implementations, the cutting device 3140 comprises an electrocautery snare or lasso 3141, wherein the snare or lasso 3141 is formed of electrodes that can be comprised of a metallic element allowing current to flow. In some implementations, the cutting device 3140 is made of nitinol to allow for shape-memory characteristics. In some implementations, the snare or lasso 3141 can be comprised of surfaces which allow for radiofrequency energy to ablate the tissue around the implantable device 3100.

The snare or lasso 3141 can serve as the cutting device 3140 and also as a stabilization component. In some implementations, a separate stabilization component 3150 can be used. In some implementations, the stabilization component 3150 can be deployed through the retrieval catheter 3160 or a separate catheter (not shown). In some implementations, the stabilization component 3150 comprises an element for grasping and stabilizing the implantable device 3100, such as a stabilizing snare 3152. The stabilizing snare 3152 can be secured around a portion of the implantable device 3100, such as the collar 3111 or the cap 3114. In some implementations, the portion of the implantable device 3100 that the stabilizing snare 3152 attaches to, such as the cap 3114 or collar 3111, can be configured (e.g., radiopaque material) to be seen under imaging (e.g., fluoroscopy, x-ray, etc.) such that it allows the user to more easily secure the stabilization component 3150 to the implantable device 3100. In some implementations, the stabilization component 3150 can comprise pincers, graspers, vacuum suction devices, or any other means of connecting or docking. In some implementations, the stabilization component 3150 stabilizes the implantable device 3100 and also allows the implantable device 3100 to be drawn up into the retrieval catheter 3160, partially into the retrieval catheter, or into abutment with the retrieval catheter.

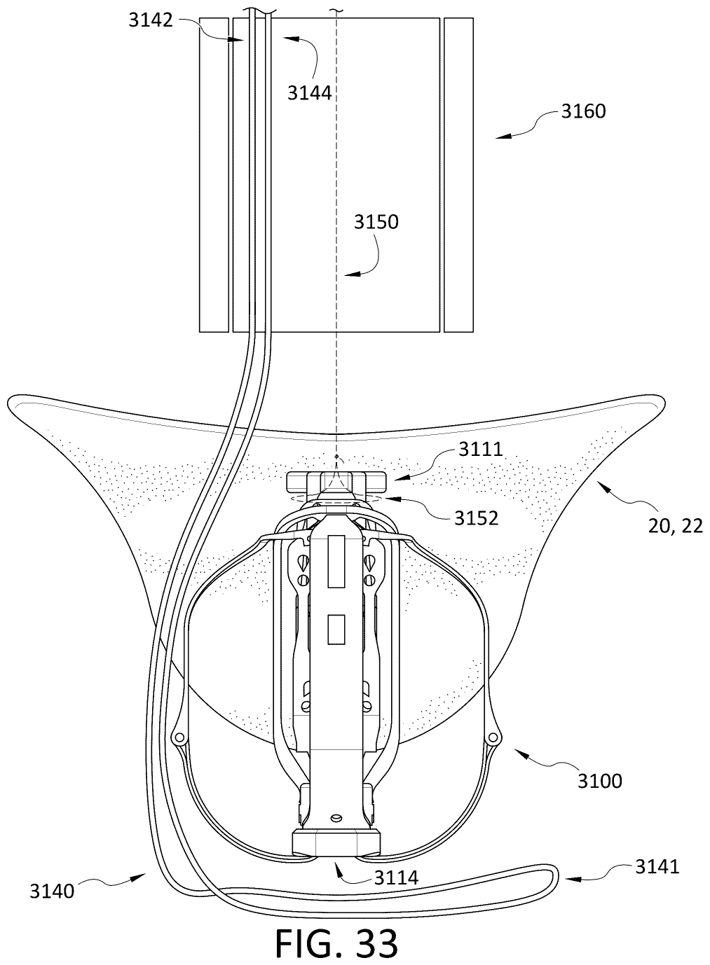

As shown in FIGS. 32-33, in some implementations the cutting device 3140 is deployed by the retrieval catheter 3160 and extended through the native heart valve between the anterior and posterior leaflets 20, 22 and alongside the implantable device, until the snare or lasso 3141 is disposed under the distal end of the implantable device 3100. Optionally, a stabilization component 3150 can be secured around a portion of the implantable device 3100, such as by placing a stabilization snare 3152 around the collar 3111, before or after the cutting device 3140 is deployed. Alternatively or additionally, the cutting device 3140 can first be used to stabilize the implantable device 3100 such as by securing the snare or lasso 3141 around a portion of the implantable device 3100, such as the cap 3114.

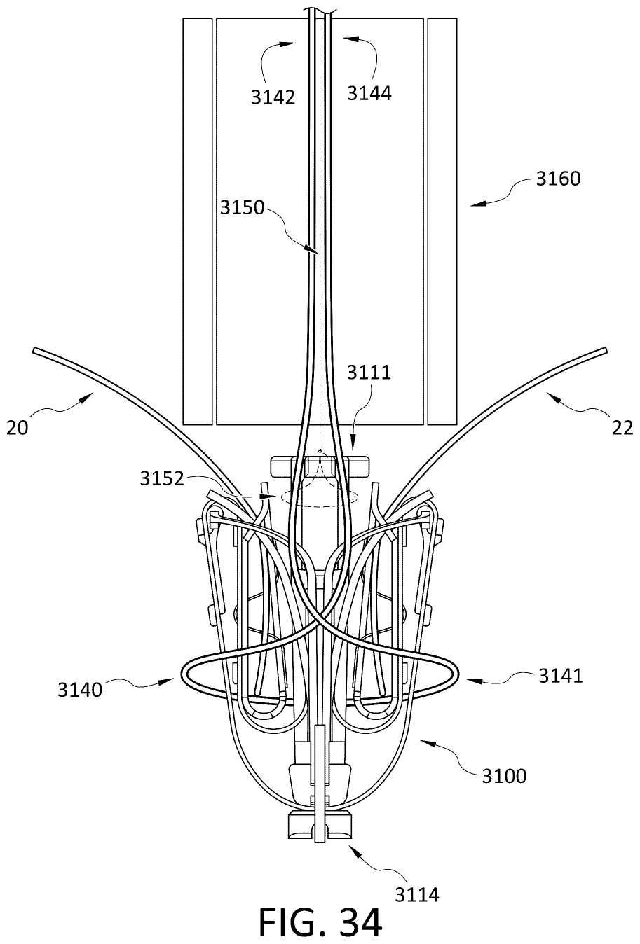

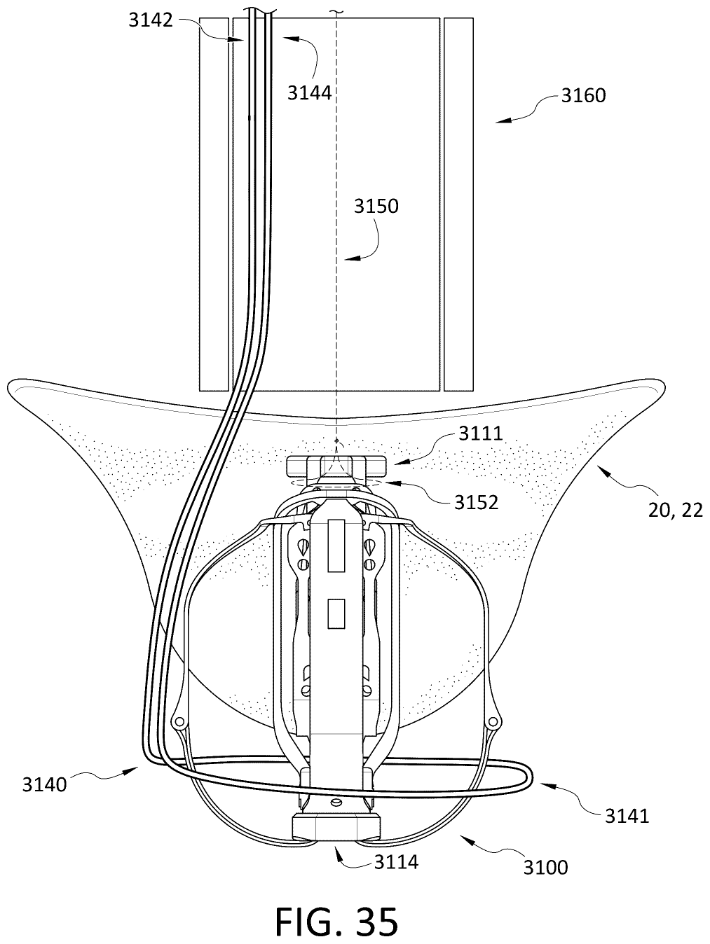

As shown in FIGS. 34 and 35, in some implementations at least one of the cutting device 3140 and the stabilization component 3150 can be used to retract the implantable device 3100 upwards towards the distal end of the retrieval catheter 3160 and/or the distal end of the retrieval catheter 3160 can be advanced toward at least one of the cutting device 3140 and the stabilization component 3150. As stated above, the distal end of the retrieval catheter 3160 can be used to further stabilize the anterior and posterior leaflets 20, 22 and/or the implantable device 3100 during resection. In some implementations, prior to resection, the implantable device 3100 is first retracted and/or the distal end of the retrieval catheter 3160 is advanced such that the anterior and posterior leaflets 20, 22 are proximate to the distal end of the retrieval catheter 3160. In some implementations, the snare or lasso 3141 is retracted upwards such that it surrounds the implantable device 3100. As the snare or lasso 3141 continues to be retracted around the implantable device 3100, the snare or lasso 3141 will come in contact with the tissue of the anterior and posterior leaflets 20, 22 on either side of and/or around the implantable device 3100. Using any of the methods of cutting or resecting described above, such as electrocautery, the snare or lasso 3141 will sever the anterior and posterior leaflets 20, 22 proximate to the implantable device 3100, such that the portion of the anterior and posterior leaflets 20, 22 grasped by the implantable device 3100 will remain attached thereto. The implantable device 3100 can then be retracted upwards towards the distal end of the retrieval catheter 3160 by at least one of the cutting device 3140 or the stabilization component 3150.

As shown in FIGS. 36 and 37, in some implementations two cutting devices 3140A, 3140B are deployed by the retrieval catheter 3160 and extended through the native heart valve between the anterior and posterior leaflets 20, 22, under the distal end of the implantable device 3100. In some implementations, the cutting devices 3140A, 3140B each comprise a snare or lasso 3141A, 3141B. The cutting devices 3140A, 3140B can each have a first end 3142A, 3142B and a second end 3144A, 3144B, wherein at least one end, and in some implementations, all ends 3142A, 3142B, 3144A, 3144B, extend proximally through the distal end of the retrieval catheter 3160 and are secured to a retrieval component or device at the proximal end of the retrieval catheter 3160, such that the user can extend or retract the cutting devices 3140A, 3140B within the retrieval catheter 3160.

In some implementations, the cutting devices 3140A, 3140B are deployed on opposite sides of the implantable device 3100. Optionally, a stabilization component 3150 can be secured around a portion of the implantable device 3100, such as by placing a stabilization snare 3152 around the collar 3111, before or after the cutting device 3140 is deployed. Alternatively or additionally, at least one of the cutting devices 3140A, 3140B can first be used to stabilize the implantable device 3100 such as by securing at least one of the snare or lasso 3141A, 3141B of each cutting device 3140 around a portion of the implantable device 3100, such as the cap 3114.

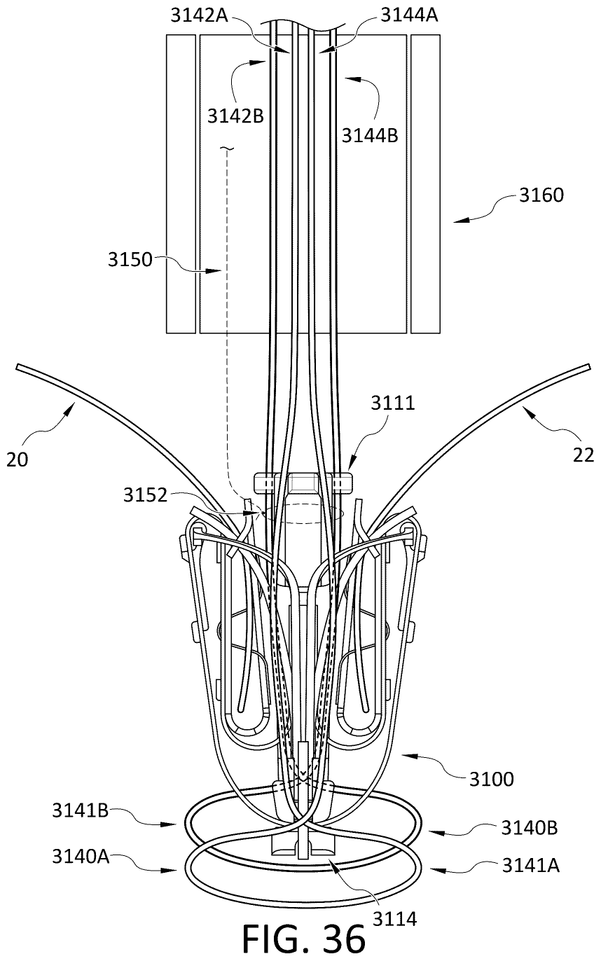

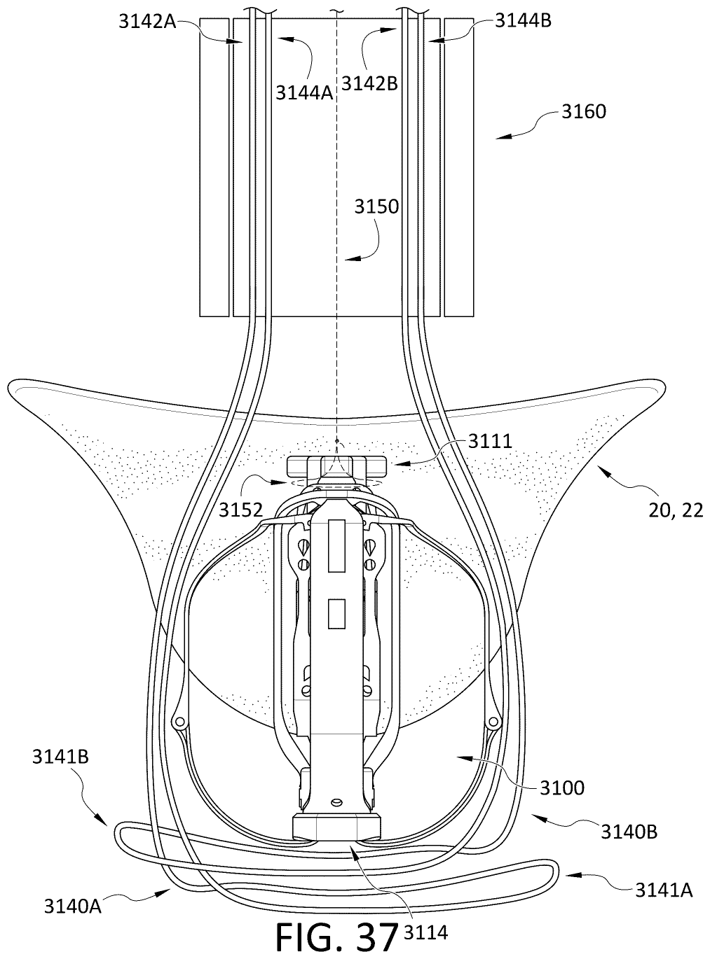

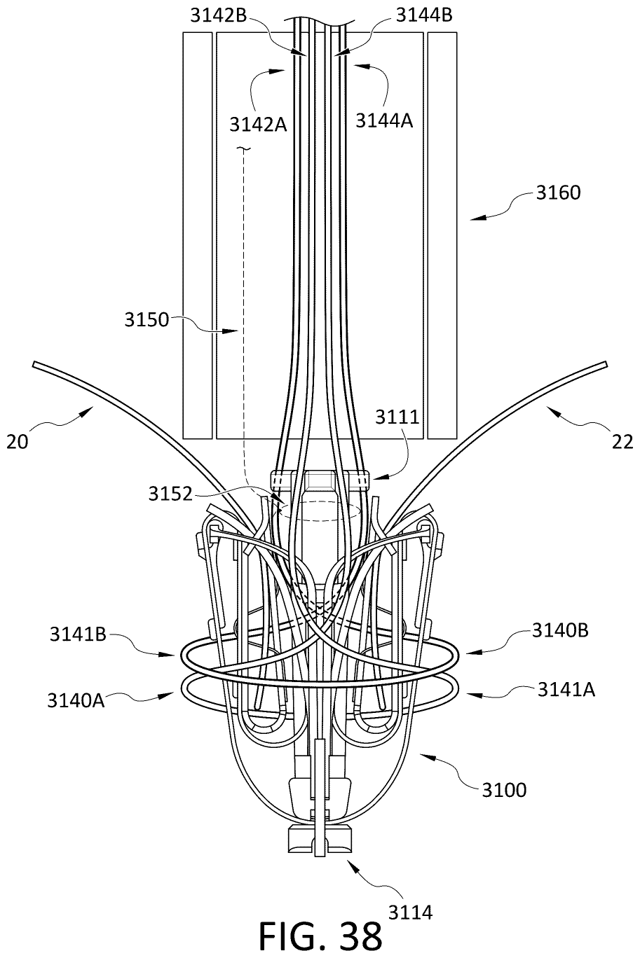

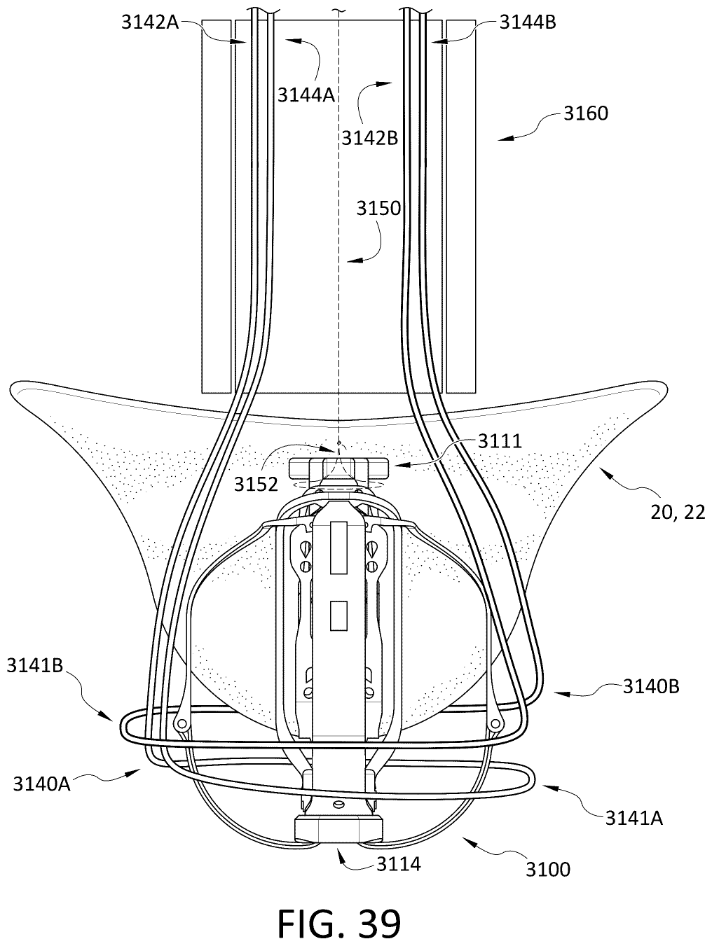

As shown in FIGS. 38 and 39, in some implementations at least one of the cutting devices 3140A, 3140B and the stabilization component 3150 can be used to retract the implantable device 3100 proximally towards the distal end of the retrieval catheter 3160 and/or the distal end of the retrieval catheter 3160 can be advanced toward at least one of the cutting device 3140 and the stabilization component 3150. As stated above, the distal end of the retrieval catheter 3160 can be used to further stabilize the anterior and posterior leaflets 20, 22 and/or the implantable device 3100 during resection. In some implementations, prior to resection, the implantable device 3100 is first retracted and/or the retrieval catheter 3160 is advanced such that the anterior and posterior leaflets 20, 22 are proximate to the distal end of the retrieval catheter 3160.

In some implementations, at least one of the snares or lassos 3141A, 3141B, are retracted upwards such that they surround the implantable device 3100. As the snares or lassos 3141A, 3141B continue to be retracted around the implantable device 3100, the snares or lassos 3141A, 3141B will come in contact with the tissue of the anterior and posterior leaflets 20, 22 on either side of the implantable device 3100. In some implementations, a first snare or lasso 3141A resects the anterior leaflet 20, and a second snare or lasso 3141B resects the posterior leaflet 22. Using any of the methods of cutting or resecting described above, such as electrocautery, the snares or lassos 3141A, 3141B will sever the anterior and posterior leaflets 20, 22 proximate to the implantable device 3100, such that the portion of the anterior and posterior leaflets 20, 22 grasped by the implantable device 3100 will remain attached thereto. The implantable device 3100 can then be retracted proximally towards the distal end of the retrieval catheter 3160 by at least one of the cutting devices 3140A, 3140B or the stabilization component 3150.

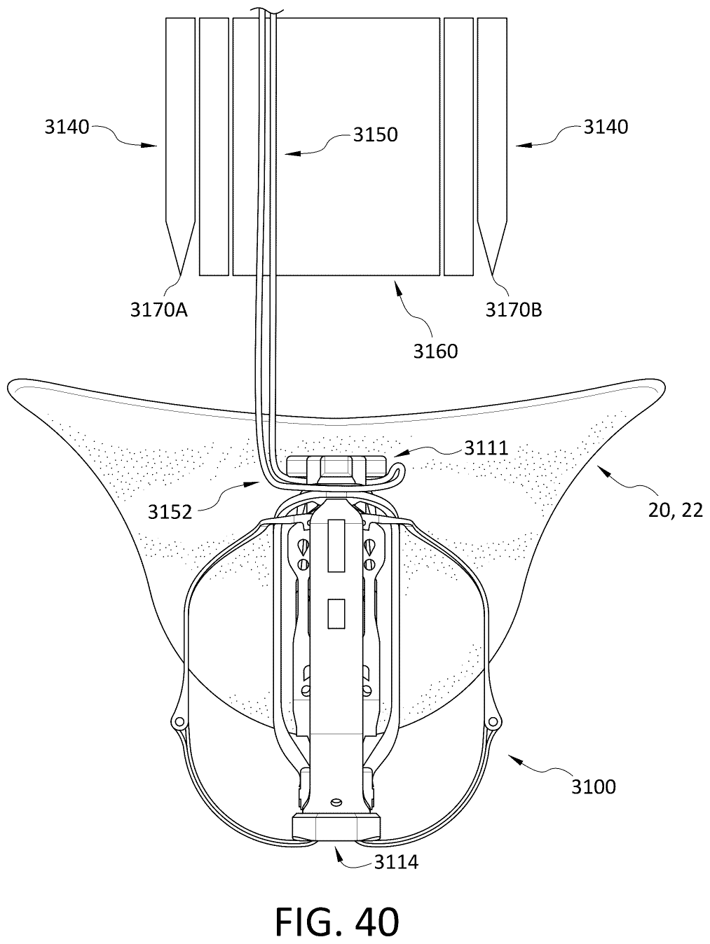

FIGS. 40-43 illustrate an example device and method for retrieving an implantable device 3100 using at least one cutting device 3140, wherein the cutting device 3140 comprises at least one of a coring element 3170A, 3170B. FIG. 40 shows a side view of an example retrieval catheter 3160, a cutting device 3140 comprising at least one coring element 3170A, 3170B, and a stabilization component 3150. As shown in FIG. 40, in some implementations the implantable device 3100 can comprise a cap 3114 at the distal end and a collar 3111 at the proximal end of the implantable device 3100. The example retrieval catheter 3160 provides the conduit through which the cutting device 3140 and stabilization component 3150 are delivered to the implantable device 3100. In some implementations, the implantable device 3100 is removed from the native heart valve via retrieval catheter 3160.

In some implementations, the distal end of the retrieval catheter 3160 can also be used to provide a downward force on the proximal side of the anterior and posterior leaflets 20, 22 and/or the implantable device 3100 in order to keep the leaflets 20, 22 taut and/or the implantable device 3100 stable during the resecting process. The inside diameter of the retrieval catheter 3160 can have an increased inner diameter such that an implantable device 3100 and portions of the dissected anterior and posterior leaflets 20, 22 can be retracted therethrough.

In some implementations, the at least one coring element 3170A, 3170B is deployed along the external surface of the retrieval catheter 3160. In some implementations, the at least one coring element 3170A, 3170B is deployed from an internal surface of the retrieval catheter 3160. In some implementations, the cutting device comprises two coring elements 3170A, 3170B deployed on opposing sides of the retrieval catheter 3160. In some implementations, the cutting device 3140 comprises a single coring element (reference numbers 3170A, 3170B represent two portions of the single coring element in this implementation) that surrounds the entirety of the retrieval catheter 3160. For example, the coring element(s) 3170A, 3170B can be annular or semi-annular or can include an annular or semi annular cutting portion. In some implementations, the coring elements 3170A, 3170B are equipped with a feature for cutting, severing, or ablating the anterior and posterior leaflets 20, 22, such as through the use of friction, electrocautery, vibration, serration, or the like. In some implementations, the at least one coring element 3170A, 3170B can be circular, arc-shaped, or annular. In some implementations, the at least one coring element 3170A, 3170B can enter the tissue of the anterior and posterior leaflets 20, 22, severing the tissue as they rotate. In some implementations, the at least one coring element 3170A, 3170B can be equipped with blades or serrations to cut the tissue of the anterior and posterior leaflets 20, 22. In some implementations, the coring elements 3170A, 3170B comprises an electrosurgical tip or blade formed of electrodes that can be comprised of a metallic element allowing current to flow. In some implementations, the coring elements 3170A, 3170B are made of nitinol to allow for shape-memory characteristics. In some implementations, the coring elements 3170A, 3170B can be comprised of surfaces which allow for radiofrequency energy to ablate the tissue around the implantable device 3100.

In some implementations, the stabilization component 3150 can be deployed through the retrieval catheter 3160 or a separate catheter (not shown). In some implementations, the stabilization component 3150 comprises an element for grasping and stabilizing the implantable device 3100, such as a stabilizing snare 3152. The stabilizing snare 3152 can be secured around a portion of the implantable device 3100, such as the collar 3111 or the cap 3114. In some implementations, the portion of the implantable device 3100 that the stabilizing snare 3152 attaches to, such as the cap 3114 or collar 3111, can be configured to enhance imaging such that it allows the user to more easily secure the stabilization component 3150 to the implantable device 3100. In some implementations, the stabilization component 3150 can comprise pincers, graspers, vacuum suction devices, or any other means of docking. In some implementations, the stabilization component 3150 stabilizes the implantable device 3100 and also allows the implantable device 3100 to be drawn up into the retrieval catheter 3160.

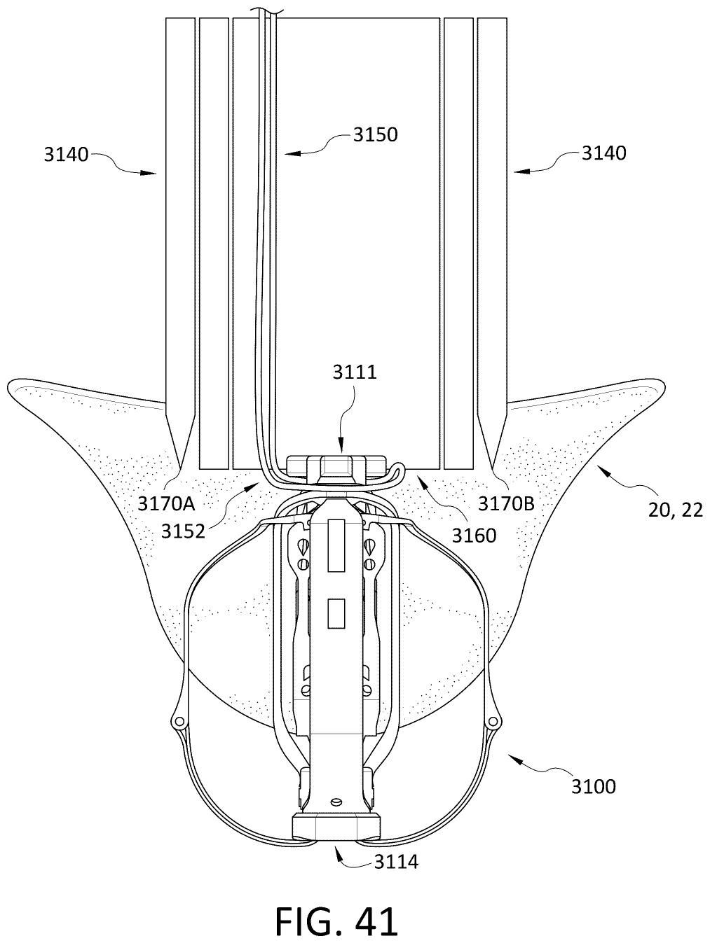

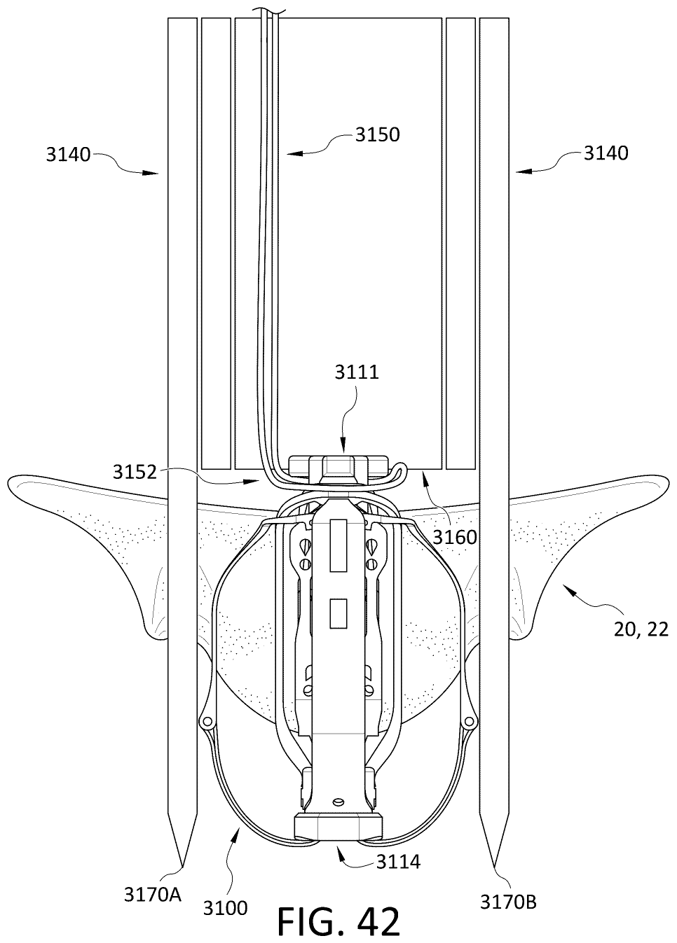

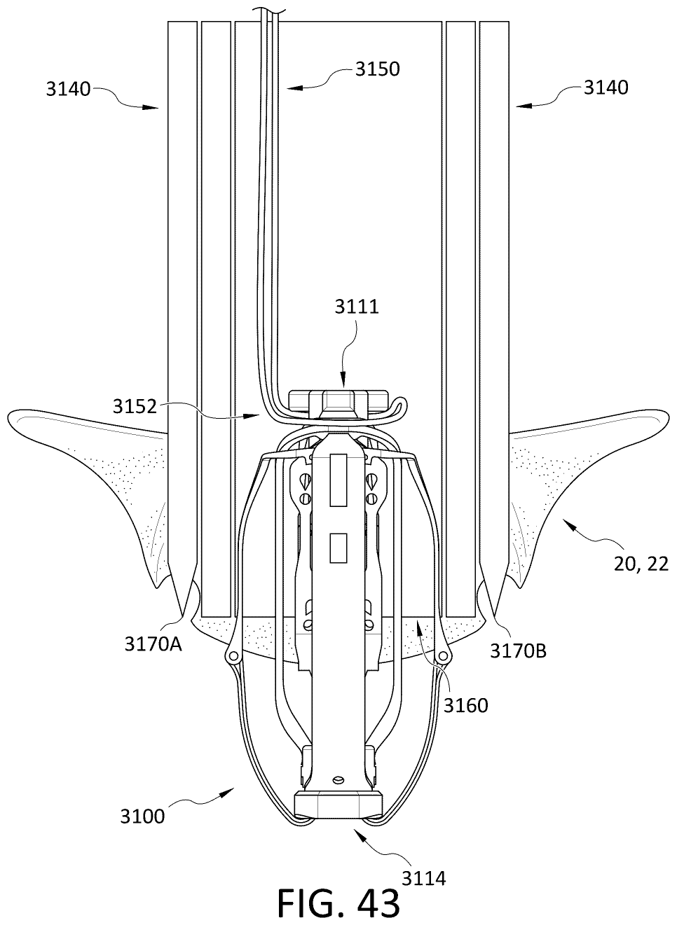

As shown in FIG. 40, in some implementations a stabilization component 3150 is secured around a portion of the implantable device 3100, such as by placing a stabilization snare 3152 around the collar 3111. As shown in FIG. 41, the stabilization component 3150 can be used to retract the implantable device 3100 proximally towards the distal end of the retrieval catheter 3160 and the coring elements 3170A, 3170B and/or the retrieval catheter 3160 and/or the coring elements 3170A, 3170B can be advanced towards the implantable device 3100. As stated above, the distal end of the retrieval catheter 3160 can be used to further stabilize the anterior and posterior leaflets 20, 22 and/or the implantable device 3100 during resection. In some implementations, prior to resection, the implantable device 3100 is first retracted such that the anterior and posterior leaflets 20, 22 are proximate to the distal end of the retrieval catheter 3160. As shown in FIG. 42, as the stabilization component 3150 continues to retract the implantable device 3100 upwards into the distal end of the retrieval catheter 3160 and/or the retrieval catheter 3160 and the coring elements 3170A, 3170B are further advanced, the coring elements 3170A, 3170B will sever the anterior and posterior leaflets 20, 22 proximate to the implantable device using any of the methods of cutting or resecting described above, such as with a sharp blade. A portion of the anterior and posterior leaflets 20, 22 grasped by the implantable device 3100 will remain attached thereto. As shown in FIG. 43, in some implementations, the coring elements 3170A, 3170B can further be deployed distally such that they can fully sever the anterior and posterior leaflets 20, 22. In some implementations, the coring elements 3170A, 3170B can move in several directions, such as longitudinally, laterally, transversally, or radially, such as in a radial path around the entirety of the implantable device 3100, to sever the implantable device 3100 completely from the anterior and posterior leaflets 20, 22. The implantable device 3100 can then be retracted upwards towards the distal end of the retrieval catheter 3160 by the stabilization component 3150.

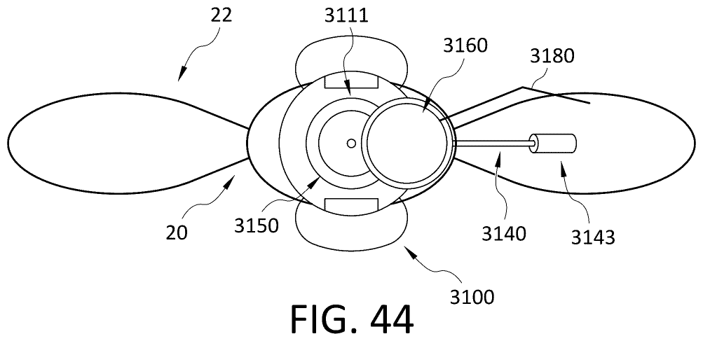

FIGS. 44-47 illustrate example devices and methods for retrieving an implantable device 3100 using at least one cutting device 3140, where the cutting device 3140 comprises a moveable cutting tip 3143, such as a blade or an electrosurgical tip. The retrieval catheter is shown as being offset from the central axis of the implantable device 3100 for the purpose of illustration. In some implementations, the retrieval catheter 3160 can be aligned directly above the proximal end of the implantable device 3100. As shown in FIG. 44, in some implementations the implantable device 3100 can comprise a collar 3111 at the proximal end of the implantable device 3100.

FIG. 44 shows a top view of an example retrieval catheter 3160, a cutting device 3140 comprising a cutting tip 3143, a stabilization component 3150, and an optional indicator or gauge 3180. The indicator or gauge 3180 can be configured to be in the same path as the moveable cutting tip but lead the moveable cutting tip 3143 to engage the tissue to be cut before the cutting tip 3143. The engagement of the tissue with the indicator or gauge 3180 can provide the user with an indication that tissue is about to be cut. In some implementations, the indicator or gauge 3180 can be configured to differentiate between different types of tissue. For example, the indicator or gauge 3180 can be configured to differentiate valve leaflet tissue from other types of tissue, such as chordae tendinea.

In some implementations, the retrieval catheter 3160 provides the conduit through which the cutting device 3140, stabilization component 3150, and the indicator or gauge 3180 are delivered to the implantable device 3100. In some other some implementations, each of the cutting device 3140, stabilization component 3150, and/or indicator or gauge 3180 can be deployed via separate catheters. In some implementations, the implantable device 3100 is removed from the native heart valve via retrieval catheter 3160. In some implementations, the distal end of the retrieval catheter 3160 can also be used to provide a downward force on the proximal side of the anterior and posterior leaflets 20, 22 and/or the implantable device 3100 in order to keep the anterior and posterior leaflets 20, 22 taut and/or the implantable device 3100 stable during the resecting process. The inside diameter of the retrieval catheter 3160 can have an increased inner diameter such that an implantable device 3100 and portions of the dissected anterior and posterior leaflets 20, 22 can be retracted therethrough.

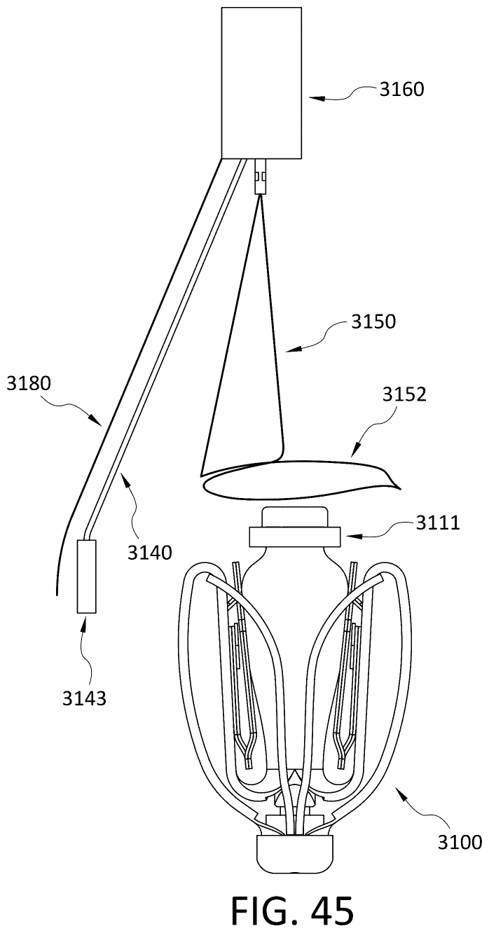

As shown in FIG. 45, in some implementations, the cutting device 3140 comprises a single cutting tip 3143 formed of electrodes that can be comprised of a metallic element allowing current to flow. In some implementations, the cutting device 3140 is made of nitinol to allow for shape-memory characteristics. In some implementations, the stabilization component 3150 and indicator or gauge 3180 can be deployed through the retrieval catheter 3160 or a separate catheter (not shown). In some implementations, the stabilization component 3150 comprises an element for grasping and stabilizing the implantable device 3100, such as a stabilizing snare 3152. The stabilizing snare 3152 can be secured around a portion of the implantable device 3100, such as the collar 3111. In some implementations, the portion of the implantable device 3100 that the stabilizing snare 3152 attaches to, such as the collar 3111, can be configured to enhance imaging such that it allows the user to more easily secure the stabilization component 3150 to the implantable device 3100. In some implementations, the stabilization component 3150 can comprise pincers, graspers, vacuum suction devices, or any other means of docking. In some implementations, the stabilization component 3150 stabilizes the implantable device 3100 and also allows the implantable device 3100 to be drawn up into the retrieval catheter 3160.

An indicator or gauge 3180 can be used prior to or concurrently with the cutting device 3140 in order to indicate the location of the anterior and posterior leaflets 20, 22, and specifically any portion of the anterior and posterior leaflets 20, 22 that remain uncut. For example, the indicator or gauge 3180 can comprise a radiopaque feature. In some implementations, the indicator or gauge 3180 can be a radiopaque feature and/or a depth gauge. In some implementations, the indicator or gauge can be made of a long, compliant wire or rod. In some implementations, the indicator or gauge 3180 will make contact with the tissue within the ventricle, including with the anterior and posterior leaflets 20, 22. This indicator or gauge 3180 can be employed in any of the examples of the devices and methods for the retrieval of an implantable device shown and described in FIGS. 31-59B. The indicator or gauge 3180 can be deployed from the same or separate catheter as the cutting device 3140 and/or the stabilization component 3150.

In some implementations, the cutting tip 3143 of the cutting device 3140 are deployed between the anterior and posterior leaflets 20, 22 parallel to the implantable device 3100. Then, the cutting tip 3143 will rotate around the implantable device 3100, severing the anterior and posterior leaflets 20, 22 proximate to the implantable device. The indicator or gauge 3180 can move along with the cutting tip 3143 to guide the severing process. A portion of the anterior and posterior leaflets 20, 22 grasped by the implantable device 3100 will remain attached thereto. In some implementations, the cutting tip 3143 can move in several directions, such as longitudinally, laterally, transversally, or radially, such as in an arcuate 360-degree path around the entirety of the implantable device 3100, to sever the implantable device 3100 completely from the anterior and posterior leaflets s 20, 22. The implantable device 3100 can then be retracted upwards towards the distal end of the retrieval catheter 3160 by the stabilization component 3150. In some implementations, the cutting tip 3143 can move in an arcuate 180-degree or similar path around enough of the implantable device 3100 to cut the implantable device 3100 off of one of the leaflets, while leaving the device attached to the other leaflet. Any of the implementations disclosed herein can be configured to cut the implantable device 3100 off of one of the leaflets while leaving the device attached to the other leaflet.

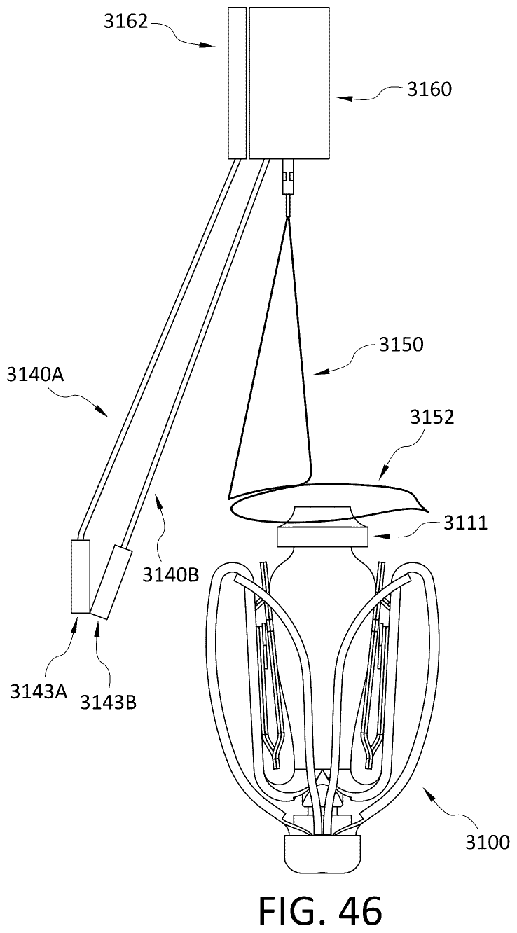

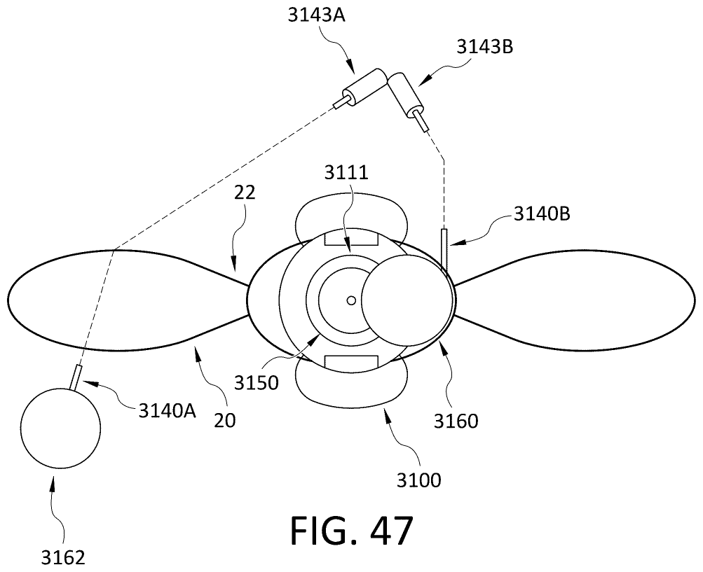

Two cutting devices 3140A, 3140B each with a cutting tip 3143A, 3143B can also be used. FIG. 47 shows a top view of an example retrieval catheter 3160, and two cutting devices 3140A, 3140B each comprising a cutting tip 3143A, 3143B and a stabilization component 3150. In some implementations, the cutting tips 3143A, 3143B are attached such that the cutting devices 3140A, 3140B form a loop or snare. An optional indicator or gauge (not shown) as described above can also be used. The retrieval catheter is shown as being offset from the central axis of the implantable device 3100 for the purposes of illustration. In some implementations, the retrieval catheter 3160 can be aligned directly above the proximal end of the implantable device 3100. As shown in FIG. 46-47, in some implementations the implantable device 3100 can comprise a collar 3111 at the proximal end of the implantable device 3100.

In some implementations, the example retrieval catheter 3160 provides the conduit through which the cutting devices 3140A, 3140B, stabilization component 3150, and the optional indicator or gauge are delivered to the implantable device 3100. In some other some implementations, each of the cutting devices 3140A, 3140B and the stabilization component 3150 can be deployed via separate catheters, such as a second catheter 3162. In some implementations, the implantable device 3100 is removed from the native heart valve via retrieval catheter 3160. In some implementations, the distal end of the retrieval catheter 3160 can also be used to provide a downward force on the proximal side of the leaflets 20, 22 and/or the implantable device 3100 in order to keep the anterior and posterior leaflets 20, 22 taut and/or the implantable device 3100 stable during the resecting process. The inside diameter of the retrieval catheter 3160 can have an increased inner diameter such that an implantable device 3100 and portions of the dissected anterior and posterior leaflets 20, 22 can be retracted therethrough.

As shown in FIG. 46, in some implementations, the cutting devices 3140A, 3140B each comprises a single cutting tip 3143A, 3143B formed of electrodes that can be comprised of a metallic element allowing current to flow. In some implementations, the cutting devices 3140A, 3140B are made of nitinol to allow for shape-memory characteristics. In some implementations, the stabilization component 3150 can be deployed through the retrieval catheter 3160 or a separate catheter 3162. In some implementations, the stabilization component 3150 comprises an element for grasping and stabilizing the implantable device 3100, such as a stabilizing snare 3152. The stabilizing snare 3152 can be secured around a portion of the implantable device 3100, such as the collar 3111. In some implementations, the portion of the implantable device 3100 that the stabilizing snare 3152 attaches to, such as the collar 3111, can be configured to enhance imaging such that it allows the user to more easily secure the stabilization component 3150 to the implantable device 3100. In some implementations, the stabilization component 3150 can comprise pincers, graspers, vacuum suction devices, or any other means of docking. In some implementations, the stabilization component 3150 stabilizes the implantable device 3100 and also allows the implantable device 3100 to be drawn up into the retrieval catheter 3160.

In some implementations, a first cutting tip 3143A of the first cutting device 3140A are deployed from the retrieval catheter 3160, into the space between the anterior and posterior leaflets 20, 22 below the center of the anterior leaflet 20 and/or above the clasp of the valve repair device that is attached to the anterior leaflet. Then (or at the same time), a second cutting tip 3143B of the second cutting device 3140B are deployed from a second catheter 3162, into a second space between the anterior and posterior leaflets 20, 22 below the center of the anterior leaflet 20 and/or above the clasp of the valve repair device that is attached to the anterior leaflet. Then, in some implementations, the cutting tips 3143A, 3143B will cut away from one another towards the outer edges of the anterior leaflet 20, each severing the anterior leaflet 20 proximate to the implantable device. In some implementations, the cutting tips 3143A, 3143B are attached to one another either before or after deployment to form a snare or loop, wherein the cutting devices 3140A, 3140B are looped around the anterior leaflet 20 using the snare formed by the cutting tips 3143A, 3143B.

In some implementations, the implantable device 3100 can remain attached to the second leaflet, such as the posterior leaflet 22, or the process described above can optionally be repeated on the second leaflet, such as the posterior leaflet 22, to sever the implantable device 3100 completely from the anterior and posterior leaflets 20, 22. In order to sever the posterior leaflet 22, each of the cutting tips 3143A, 3143B can be retracted upwards into the ventricle and then deployed at the posterior leaflet 22. In some implementations, the cutting devices 3140A, 3140B can remain attached via the cutting tips 3143A, 3143B and thus looped around and underneath the posterior leaflet 22, wherein the posterior leaflet 22 would be severed by moving the cutting tips 3143A, 3143B upwards towards the distal end of the retrieval catheter 3160. A portion of the anterior and posterior leaflets 20, 22 grasped by the implantable device 3100 will remain attached thereto. In some implementations, the cutting tips 3143A, 3143B can move in several directions, such as longitudinally, laterally, transversally, and/or radially, to sever the implantable device 3100 from one or both of the leaflets. If severed from both leaflets, the implantable device 3100 can be retracted upwards towards the distal end of the retrieval catheter 3160 by the stabilization component 3150.

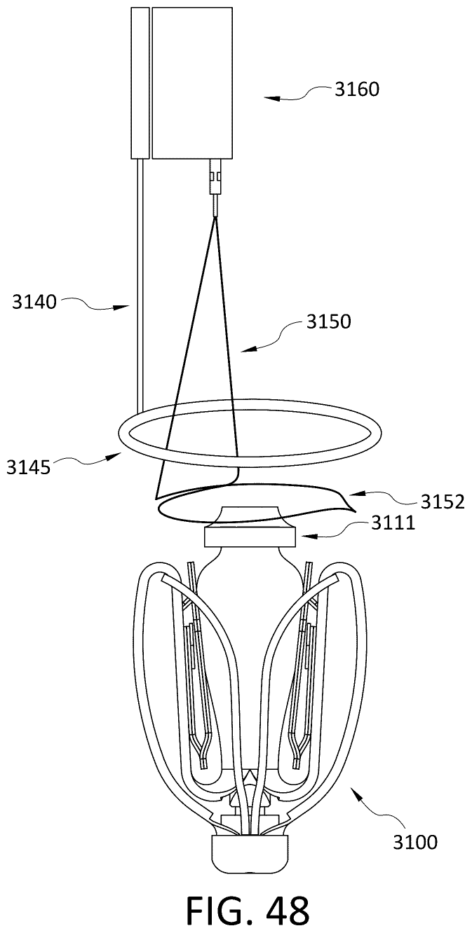

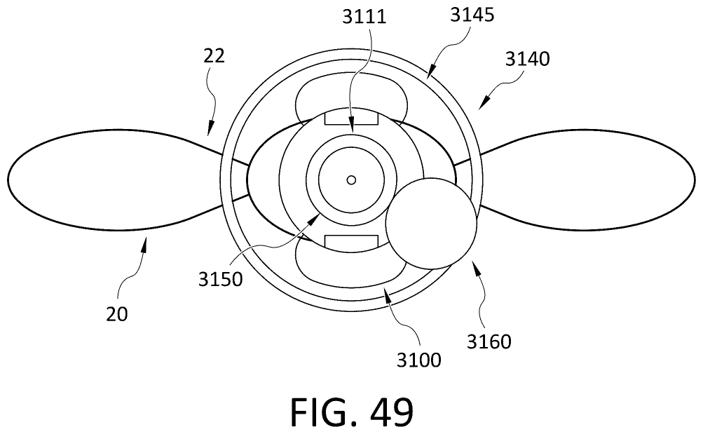

FIG. 49 shows a top view of an example retrieval catheter 3160, a cutting device 3140 comprising an electrosurgical ring 3145, a stabilization component 3150, and an optional indicator or gauge (not shown). The retrieval catheter is shown as being offset from the central axis of the implantable device 3100 for the purposes of illustration. In some implementations, the retrieval catheter 3160 can be aligned directly above the proximal end of the implantable device 3100. As shown in FIGS. 48-49, in some implementations the implantable device 3100 can comprise a collar 3111 at the proximal end of the implantable device 3100. In some implementations, the example retrieval catheter 3160 provides the conduit through which the cutting device 3140, stabilization component 3150, and the indicator or gauge are delivered to the implantable device 3100. In some other some implementations, each of the cutting device 3140, stabilization component 3150, and indicator or gauge can be deployed via separate catheters, such as a second catheter 3162. In some implementations, the implantable device 3100 is removed from the native heart valve via retrieval catheter 3160. In some implementations, the distal end of the retrieval catheter 3160 can also be used to provide a downward force on the proximal side of the leaflets 20, 22 and/or the implantable device 3100 in order to keep the leaflets 20, 22 taut and/or the implantable device 3100 stable during the resecting process. The inside diameter of the retrieval catheter 3160 can have an increased inner diameter such that an implantable device 3100 and portions of the dissected anterior and posterior leaflets 20, 22 can be retracted therethrough.

As shown in FIG. 48, in some implementations, the cutting device 3140 comprises an electrosurgical ring 3145 formed of one or more electrodes that can be comprised of a metallic element allowing current to flow. In some implementations, the cutting device 3140 is made of nitinol to allow for shape-memory characteristics. In some implementations, the stabilization component 3150 and indicator or gauge can be deployed through the retrieval catheter 3160, and the electrosurgical ring 3145 can be deployed using an optional second catheter 3162. In some implementations, the stabilization component 3150 comprises an element for grasping and stabilizing the implantable device 3100, such as a stabilizing snare 3152. The stabilizing snare 3152 can be secured around a portion of the implantable device 3100, such as the collar 3111. In some implementations, the portion of the implantable device 3100 that the stabilizing snare 3152 attaches to, such as the collar 3111, can be enhanced for imaging such that it allows the user to more easily secure the stabilization component 3150 to the implantable device 3100. In some implementations, the stabilization component 3150 can comprise pincers, graspers, vacuum suction devices, or any other means of docking. In some implementations, the stabilization component 3150 stabilizes the implantable device 3100 and also allows the implantable device 3100 to be drawn up into the retrieval catheter 3160.

In some implementations, the electrosurgical ring 3145 of the cutting device 3140 are deployed distally towards the anterior and posterior leaflets 20, 22 directly above the implantable device 3100. When the electrosurgical ring 3145 makes contact with the anterior and posterior leaflets 20, 22 the electrosurgical ring 3145 can be made to sever the anterior and posterior leaflets 20, 22 radially around the implantable device 3100. A portion of the anterior and posterior leaflets 20, 22 grasped by the implantable device 3100 will remain attached thereto. In some implementations, the electrosurgical ring 3145 can move in several directions, such as longitudinally, laterally, transversally, or radially to sever the implantable device 3100 completely from the anterior and posterior leaflets 20, 22. The implantable device 3100 can then be retracted upwards towards the distal end of the retrieval catheter 3160 by the stabilization component 3150.

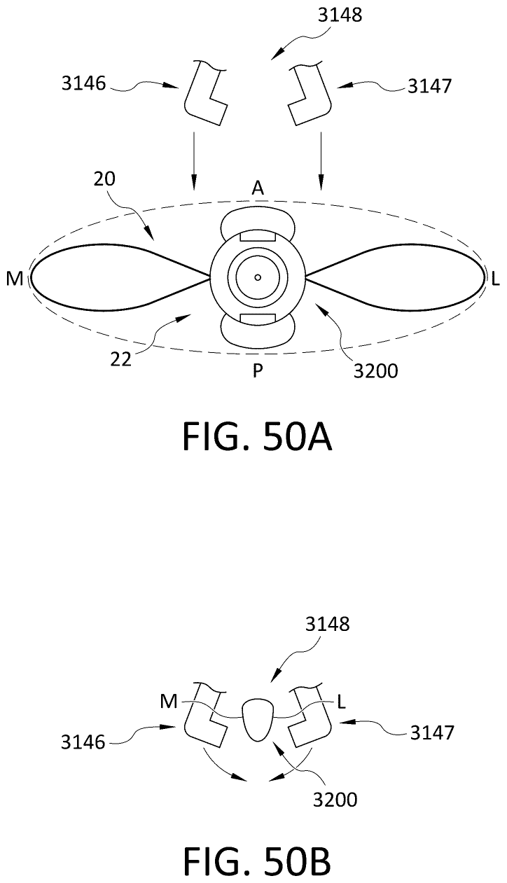

FIGS. 50A-50C illustrate example devices and methods for retrieving an implantable device 3200 using a clamp 3148 with a first grasping arm 3146 and a second grasping arm 3147. FIG. 50A shows the first and second grasping arms 3146, 3147 of a clamp 3148, and an implantable device 3200 secured to the anterior and posterior leaflets 20, 22. In some implementations, each grasping arm 3146, 3147 has a c-shaped profile and is substantially cup-shaped or shell-shaped, such that when the clamp 3148 closes, the two grasping arms 3146, 3147 form a complete encapsulation with a void in the center thereof to secure the implantable device 3200 during removal. The clamp 3148 can be equipped with various features for cutting, severing, and/or resecting the anterior and posterior leaflets 20, 22, such as serrated edges, blades, electrocautery, ultra-sonic, mechanical vibration, or friction. In some implementations, the grasping arms 3146, 3147 are equipped with serrated edges or blades to cut through the anterior and posterior leaflets 20, 22 like scissors. In some implementations, the grasping arms 3146, 3147 will each be at least partially formed of electrodes that can be comprised of a metallic element allowing current to flow, such that the grasping arms 3146, 3147 form electrocautery tools that will remove the tissue of the anterior and posterior leaflets 20, 22 as the grasping arms 3146, 3147 close around the implantable device 3200.

As shown in FIGS. 50A and 50B, the first and second grasping arms 3146, 3147 will each extend into the spaces formed by the anterior and posterior leaflets 20, 22 on either side of the implantable device 3200. In some implementations, the clamp 3148 can be guided into position using a variety of methods, such as an indicator or gauge described above, or echocardiography. In some implementations, the grasping arms 3146, 3147 are aligned to the implantable device 3200 using fluoroscopy to ensure the implantable device 3200 is positioned between the grasping arms 3146, 3147 prior to cutting the anterior and posterior leaflets 20, 22. As shown in FIGS. 50B-50C, once the grasping arms 3146, 3147 are in position around the implantable device 3200, the grasping arms 3146, 3147 will move towards one another, simultaneously cutting through the tissue of the anterior and posterior leaflets 20, 22 using one of the methods described above. Once the clamp 3148 is closed, and the grasping arms 3146, 3147 have come in contact with one another, the implantable device 3200 can be completely encapsulated within the clamp 3148 such that the implantable device 3200 is securely stored after it is completely severed from the anterior and posterior leaflets 20, 22. In some implementations, the grasping arms 3146, 3147 move in a medial-lateral direction, each cutting both the anterior and posterior leaflets 20, 22 as the clamp 3148 moves to the closed position.

In some implementations, the grasping arms 3146, 3147 equipped with blades will also be equipped with a blade guard or sheath that can cover the blades while the grasping arms 3146, 3147 are placed in position, preventing, or inhibiting any inadvertent resecting from occurring. Once the grasping arms 3146, 3147 are positioned correctly, the sheath (not shown) can be removed and the grasping arms 3146, 3147 can close around the implantable device 3200, severing the anterior and posterior leaflets 20, 22. The sheath can cover the entire clamp 3148, or each grasping arm 3146, 3147 individually.

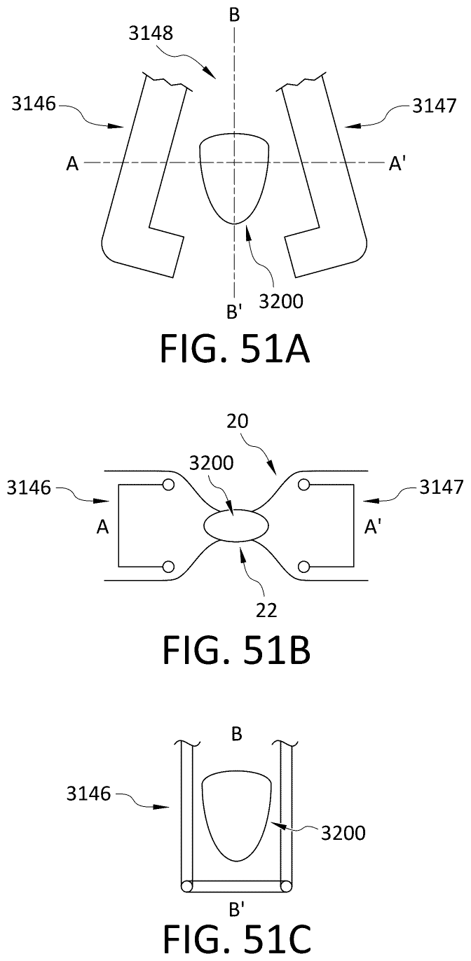

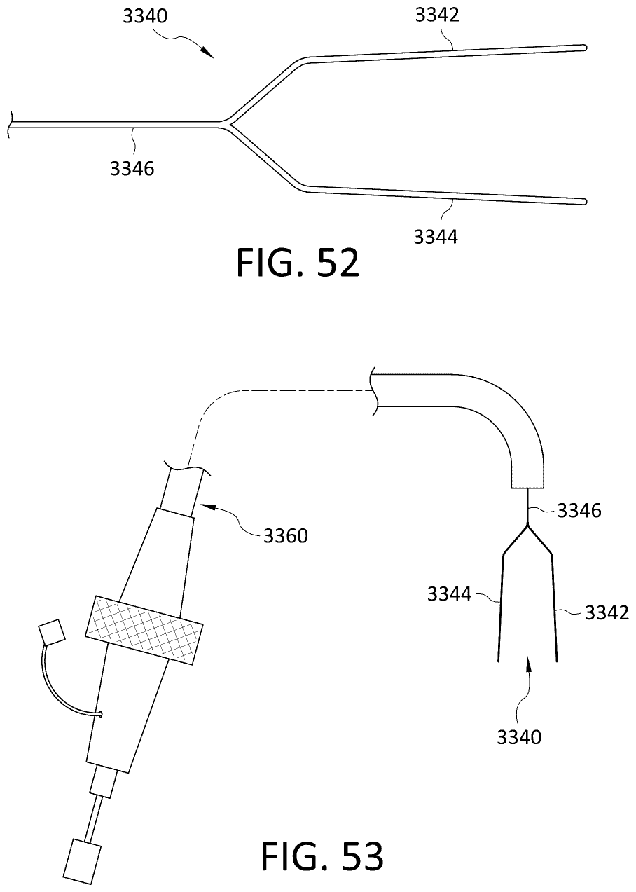

FIG. 51A shows a front view of the clamp 3148, the first grasping arm 3146 and the second grasping arm 3147, wherein an implantable device 3200 is positioned within the void space between the grasping arms 3146, 3147. FIG. 51B shows a top view along cross-section A-A′ of the implantable device 3200 secured to the anterior and posterior leaflets 20, 22 wherein the grasping arms 3146, 3147 are located within the spaces between the anterior and posterior leaflets 20, 22. FIG. 51C shows a side view of a grasping arm 3146 along cross-section B-B′ showing the implantable device 3200 encapsulated within the grasping arm 3146. FIGS. 52-56 illustrate example devices and methods for retrieving an implantable device using a cutting device 3340 shaped like a tuning fork. FIG. 52 shows a cutting device 3340 comprising a central wire 3346, a first prong 3342 and a second prong 3344. In some implementations, the first and second prongs 3342, 3344 are equipped with a feature for cutting, severing, or ablating the anterior and posterior leaflets 20, 22, such as through the use of friction, heat, electrocautery, vibration, blades, serration, or the like. In some implementations, the first and second prongs 3342, 3344 are formed of extremely sharp blades. In some implementations, the first and second prongs 3342, 3344 are formed of electrodes that can be comprised of a metallic element allowing current to flow. In some implementations, the cutting device 3340 is connected to an infrared generator, such that the heat can be used to sever the native leaflets. In some implementations, the cutting device 3340 is made of nitinol or spring wire to allow for shape-memory characteristics. In some implementations, the cutting device 3340 can be comprised of surfaces which allow for radiofrequency energy to ablate the tissue around the implantable device 3300. The grasping arms 3146, 3147 are closed to cut through the leaflets and retain the detached implantable device.

FIGS. 52 and 53 illustrate a system for removing a valve repair device 3300 from one or more leaflets 20, 22 of a native heart valve. In the implementation illustrated by FIGS. 53, a cutting device 3340 is delivered from a steerable sheath, such as a retrieval catheter 3360 that is controlled by the attached control handle. The control handle can be used to advance the catheter 3360, steer the catheter, advance the cutting device 3340 relative to the catheter, and/or control the cutting device 3340. In some implementations, the cutting device 3340 is deployed to the heart valve via the retrieval catheter 3360. In some implementations, a stabilization component, as described above, can also be deployed through the retrieval catheter 3360 or a separate catheter (not shown). The example retrieval catheter 3360 provides the conduit through which the cutting device 3340 and optional stabilization component (not shown) are delivered to an implantable device.

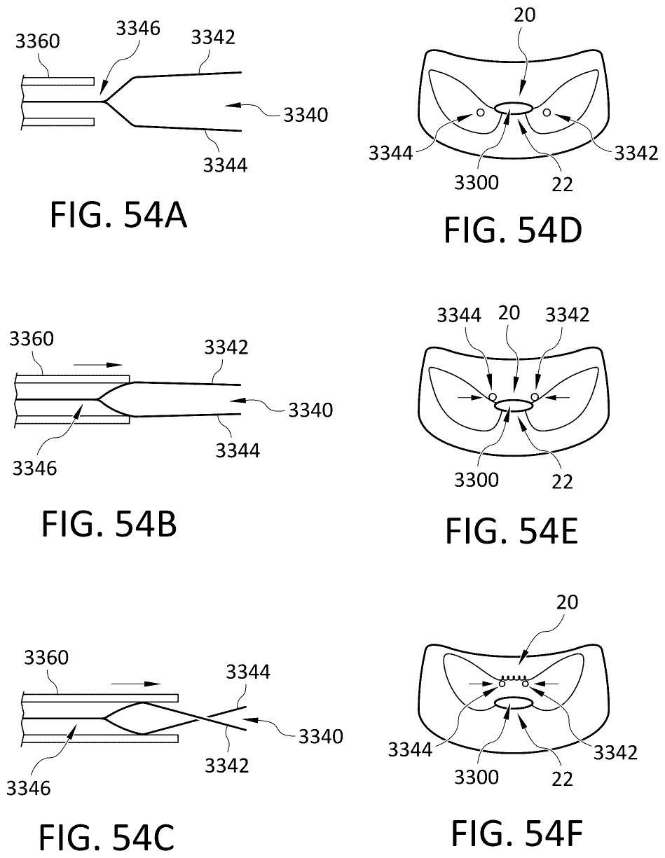

FIGS. 54A-54F illustrate operation of the cutting device 3340 with the catheter 3360 and/or the control handle. FIG. 54A is a side view of the cutting device 3340 in an extend position, wherein the first and second prongs 3342, 3344 are outside the retrieval catheter 3360, but at least a portion of the central wire 3346 remains within the retrieval catheter 3360. FIG. 54B is a side view of the cutting device 3340 in a closed position, wherein the retrieval catheter 3360 is extended distally towards the confluence of the central wire 3346 and the first and second prongs 3342, 3344, and/or the cutting device 3340 is retracted within the retrieval catheter 3360, such that the first and second prongs 3342, 3344 are forced closer together by the walls of the retrieval catheter 3360. FIG. 54C is a side view of the cutting device 3340 in a scissored position, wherein the retrieval catheter 3360 is further extended distally over a portion of the first and second prongs 3342, 3344 and/or the cutting device 3340 is further retracted within the retrieval catheter 3360, such that the first and second prongs 3342, 3344 are forced into a crisscrossed, traversed, or scissored position.

FIGS. 54D-54F illustrate how the positions of the cutting device 3340 shown in FIGS. 54A-54C function to sever the implantable device 3300 from the anterior and posterior leaflets 20, 22. FIG. 54D shows a top view of an implantable device 3300 secured to the anterior and posterior leaflets 20, 22. The cutting device 3340 can be deployed such that the first and second prongs 3342, 3344 each enter one of the openings formed by the anterior and posterior leaflets 20, 22 on either side of the implantable device 3300. As shown in FIG. 54E, as the cutting device 3340 enters the closed position, the first and second prongs 3342, 3344 move inwards from the openings towards one of the anterior and posterior leaflets 20, 22, such as the anterior leaflet 20. Once the first and second prongs 3342, 3344 make contact with the sides of the anterior leaflet 20, the cutting device 3340 begins to sever the tissue thereof using any one of the methods for severing the tissue described above, such as via electrocautery, infrared, or radiofrequency ablation. As shown in FIG. 54F, as the retrieval catheter 3360 continues to advance over the cutting device 3340, the first and second prongs 3342, 3344 traverse one another, completely severing the anterior leaflet 20.

The process shown in FIGS. 54D-54E can then be repeated on the posterior leaflet 22. In some implementations, the implantable device 3300 is removed from the native heart valve via retrieval catheter 3360. In some implementations, the distal end of the retrieval catheter 3360 can also be used to provide a downward force on the proximal side of the anterior and posterior leaflets 20, 22 and/or the implantable device 3300 in order to keep the anterior and posterior leaflets 20, 22 taut and/or the implantable device 3300 stable during the resecting process. The inside diameter of the retrieval catheter 3160 can have an increased inner diameter such that an implantable device 3100 and portions of the dissected anterior and posterior leaflets 20, 22 can be retracted therethrough.

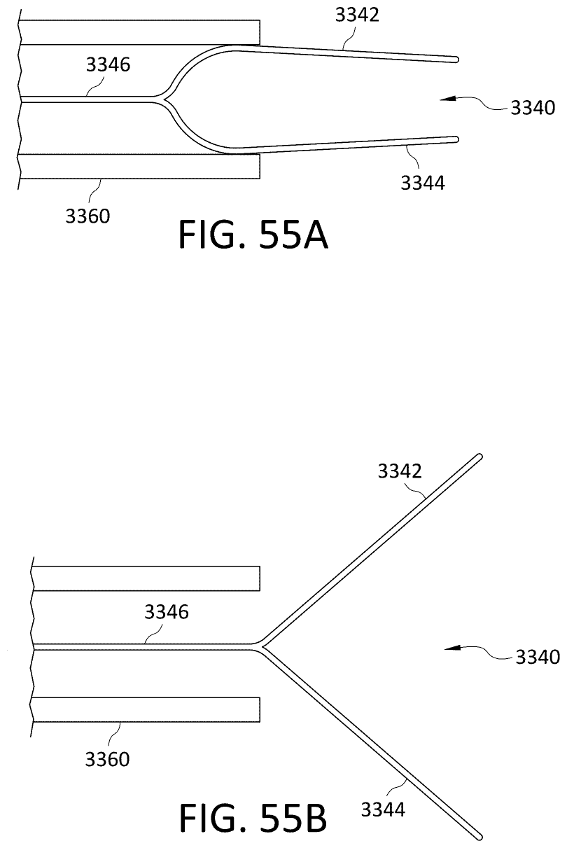

Alternatively, as shown in FIGS. 55A-55B, in some implementations the cutting device 3340 can sever the anterior and posterior leaflets 20, 22 by moving the first and second prongs 3342, 3344 away from one another instead of towards one another. FIG. 55A is a side view of the cutting device 3340 in a closed position, wherein the retrieval catheter 3360 is extended distally towards the confluence of the central wire 3346 and the first and second prongs 3342, 3344, and/or the cutting device 3340 is retracted within the retrieval catheter 3360, such that the first and second prongs 3342, 3344 are forced closer together by the walls of the retrieval catheter 3360. FIG. 55B is a side view of the cutting device 3340 in an extended position, wherein the first and second prongs 3342, 3344 are outside the retrieval catheter 3360, but at least a portion of the central wire 3346 remains within the retrieval catheter 3360.

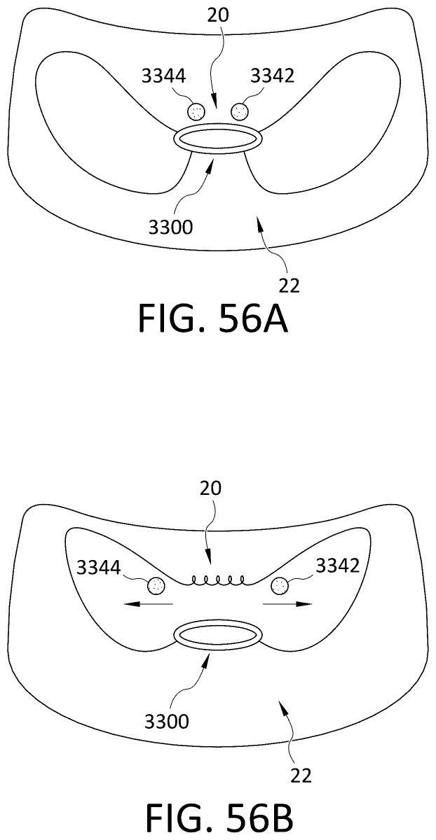

FIGS. 56A and 56B illustrate how the positions of the cutting device 3340 shown in FIGS. 55A-55B function to sever the implantable device 3300 from the anterior and posterior leaflets 20, 22. FIG. 56A shows a top view of an implantable device 3300 secured to the anterior and posterior leaflets 20, 22. The cutting device 3340 can be deployed such that the first and second prongs 3342, 3344 each enter the center of one of the anterior and posterior leaflets 20, 22 in the closed position, such as the anterior leaflet 20. A space is shown between the first and second prongs 3342, 3344 to illustrate the presence of two prongs. However, in some implementations, the two prongs can contact one another, cross each other and/or form a single hole in the leaflet tissue, such that there is no tissue bridge or only a small/tearable tissue bridge between the prongs.

As shown in FIG. 56B, as the cutting device 3340 moves to the extended position, the first and second prongs 3342, 3344 move outwards from the center of the anterior leaflet 20 towards the edges of the anterior leaflet 20. Once the first and second prongs 3342, 3344 make contact with the sides of the anterior leaflet 20, the cutting device 3340 will have severed the tissue thereof using any one of the methods for severing the tissue described above, such as via electrocautery, infrared, or radiofrequency ablation.

In some implementations, a balloon (not shown) can be inserted into the perforation in the anterior and posterior leaflets 20, 22 caused by the first and second prongs 3342, 3344. The balloon can be inflated within the perforation, passively tearing the anterior and posterior leaflets 20, 22 as it inflates. The balloon (not shown) can also be equipped with a feature for cutting, severing, or ablating the tissue such as through the use of electrocautery, vibration, blades, heat, or the like.

The process shown in FIGS. 56A-56B can then be repeated on the posterior leaflet 22. In some implementations, the implantable device 3300 is removed from the native heart valve via retrieval catheter 3360. In some implementations, the distal end of the retrieval catheter 3360 can also be used to provide a downward force on the proximal side of the anterior and posterior leaflets 20, 22 and/or the implantable device 3300 in order to keep the anterior and posterior leaflets 20, 22 taut and/or the implantable device 3300 stable during the resecting process. The inside diameter of the retrieval catheter 3160 can have an increased inner diameter such that an implantable device 3100 and portions of the dissected anterior and posterior leaflets 20, 22 can be retracted therethrough.

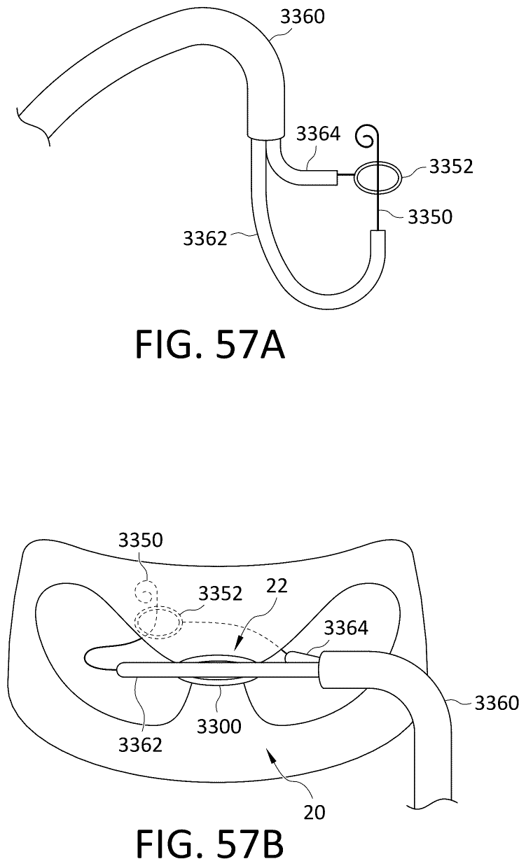

FIGS. 57A-57B illustrate an example device and method for removing an implantable device using a hook 3350 and loop 3352. As shown in FIG. 57A, an example device comprises a retrieval catheter 3360. Disposed in the retrieval catheter are a first catheter 3362 through which a hook 3350 is deployed and a second catheter 3364 through which a loop is deployed. In some implementations, the hook 3350 and loop 3352 are equipped with a feature for cutting, severing, or ablating the anterior and posterior leaflets 20, 22, such as through the use of friction, heat, electrocautery, vibration, blades, serration, or the like. In some implementations, the hook 3350 and loop 3352 are formed of electrodes that can be comprised of a metallic element allowing current to flow. In some implementations, at least one of the hook 3350 and loop 3352 is connected to an infrared generator, such that heat can be used to sever the anterior and posterior leaflets 20, 22. In some implementations, at least one of the hook 3350 and loop 3352 is made of nitinol or spring wire to allow for shape-memory characteristics. In some implementations, at least one of the hook 3350 and loop 3352 can be comprised of surfaces which allow for radiofrequency energy to ablate the tissue around the implantable device 3300. In some implementations, the shape memory properties of the hook 3350 and loop 3352 are such that when the hook 3350 is deployed from the first catheter it aligns perpendicularly to the loop 3352 when it deploys from the second catheter 3364, wherein the hook 3350 is aligned to enter the loop 3352 creating a loop or lasso that can be used to sever the anterior and posterior leaflets 20, 22.

FIG. 57B shows an implantable device 3300 secured to the anterior and posterior leaflets 20, 22. In some implementations, a hook 3350 is first deployed from a retrieval catheter 3360, or from the first catheter 3362 disposed therein, to an opening between the anterior and posterior leaflets 20, 22. Then, a loop 3352 is deployed from the retrieval catheter 3360, or from the second catheter 3364 disposed therein through the opposite opening between the anterior and posterior leaflets 20, 22. The hook 3350 then enters the loop 3352 below the anterior and posterior leaflets 20, 22 and the implantable device 3300, forming a loop or lasso around one of the native leaflets, such as the anterior leaflet 20. Then, the hook 3350 and loop 3352 can be simultaneously retracted into their respective catheters 3360, 3362, 3364, causing the loop or lasso formed by the hook 3350 and loop 3352 to make contact with the side of the anterior leaflet 20. The hook 3350 and loop 3352 will sever the tissue thereof using any one of the methods described above, such as via electrocautery, infrared, or radiofrequency ablation. This process can then be repeated on the posterior leaflet 22.

In some implementations, the implantable device 3300 is removed from the native heart valve via retrieval catheter 3360. In some implementations, the distal end of the retrieval catheter 3360 can also be used to provide a downward force on the proximal side of the anterior and posterior leaflets 20, 22 and/or the implantable device 3300 in order to keep the anterior and posterior leaflets 20, 22 taut and/or the implantable device 3300 stable during the resecting process.

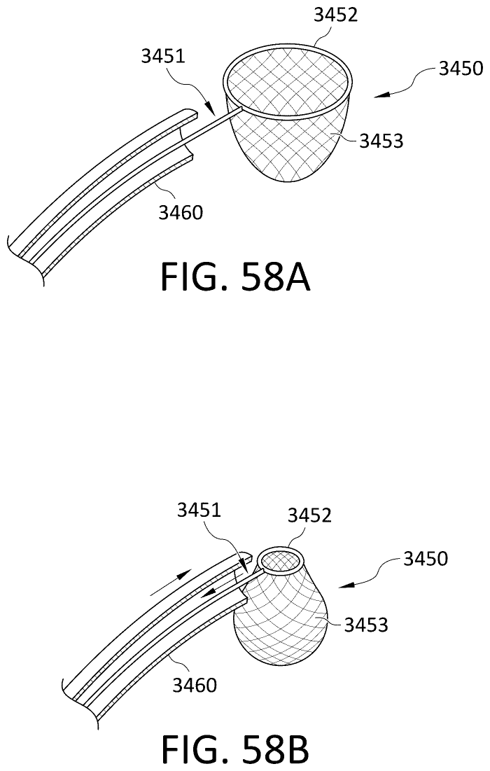

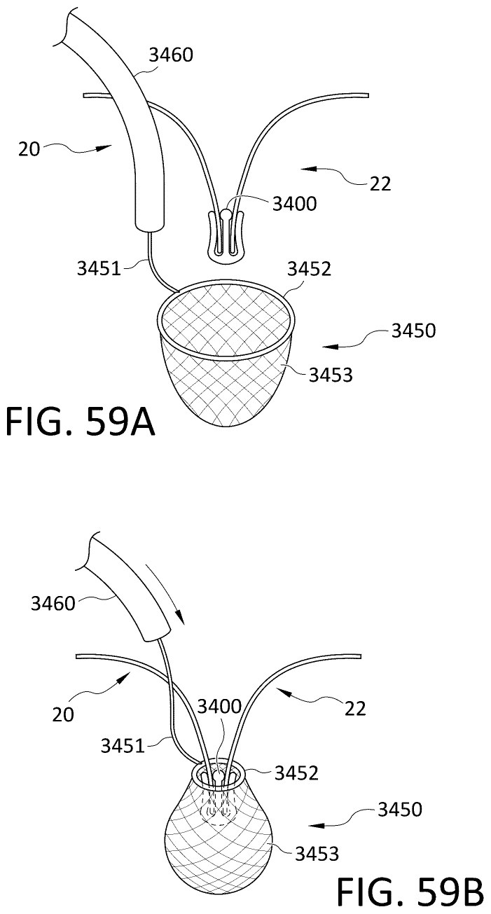

FIGS. 58A-59B illustrate an example device and method for the removal of an implantable device using a snare bag. FIG. 58A shows a retrieval catheter 3460 and a retrieval device 3450 disposed therethrough. The retrieval device 3450 can comprise a positioning element 3451 (e.g., a wire, rod, line, pusher, tether, etc.), a snare 3452, and/or a bag 3453. In some implementations, a bag 3453 can be mesh, netted, or solid material.

In some implementations, the retrieval device (e.g., snare 3452, etc.) is equipped with a feature for cutting, severing, or ablating the anterior and posterior leaflets 20, 22, such as through the use of friction, heat, electrocautery, vibration, blades, serration, or the like. In some implementations, the retrieval device (e.g., snare 3452, etc.) is formed of electrodes that can be comprised of a metallic element allowing current to flow. In some implementations, the retrieval device (e.g., snare 3452, etc.) is connected to an infrared generator, such that the heat can be used to sever the anterior and posterior leaflets 20, 22. In some implementations, at least one of a snare 3452 and a bag 3453 of the retrieval device is made of nitinol or spring wire to allow for shape-memory characteristics. In some implementations, the retrieval device (e.g., snare 3452, etc.) can be comprised of surfaces which allow for radiofrequency energy to ablate the tissue around an implantable device. As shown in FIG. 58B, the retrieval device (e.g., snare 3452, etc.) can be moved from the open to a closed position by retracting the positioning element 3451 and/or extending the retrieval catheter 3460.

FIGS. 59A and 59B illustrate an example method of using the retrieval device 3450 to remove an implantable device 3400 secured to native leaflets (e.g., the anterior and posterior leaflets 20, 22, etc.). As shown in FIG. 59A, the retrieval device 3450 can be deployed via the retrieval catheter 3460 into one of the openings between the leaflets (e.g., between the anterior and posterior leaflets 20, 22), such that the snare 3452 and bag 3453 can be positioned directly below the distal end of the implantable device 3400.

In some implementations, the positioning element 3451 is made of nitinol such that it can have shape memory properties to position the retrieval device 3450 properly once the retrieval catheter 3460 is retracted and/or the positioning element 3451 is extended therefrom.

In some implementations, as shown in FIG. 59B, once the retrieval device 3450 is in place, the retrieval catheter 3460 can then be extended distally towards the retrieval device 3450 and/or the positioning element 3451 can be retracted into the retrieval catheter 3460, such that the retrieval device 3450 moves upwards to encompass the implantable device 3400 within the bag 3453. Once the implantable device 3400 is completely within the bag 3453, the positioning element 3451 can be further retracted within the retrieval catheter 3460, causing the snare 3452 to cinch into a closed position. As the snare 3452 closes, it comes in contact with the anterior and posterior leaflets 20, 22 and severs the tissue thereof using any one of the methods described above, such as via electrocautery, infrared, or radiofrequency ablation. Once the anterior and posterior leaflets 20, 22 are completely severed and the snare 3452 is completely closed, the implantable device 3400 are enclosed securely within the retrieval device 3450 and can then be removed from the native heart valve via retrieval catheter 3460. In some implementations, the distal end of the retrieval catheter 3360 can also be used to provide a downward force on the proximal side of the anterior and posterior leaflets 20, 22 and/or the implantable device 3400 in order to keep the anterior and posterior leaflets 20, 22 taut and/or the implantable device 3400 stable during the resecting process. The inside diameter of the retrieval catheter 3160 can have an increased inner diameter such that an implantable device 3100 and portions of the dissected anterior and posterior leaflets 20, 22 can be retracted therethrough.

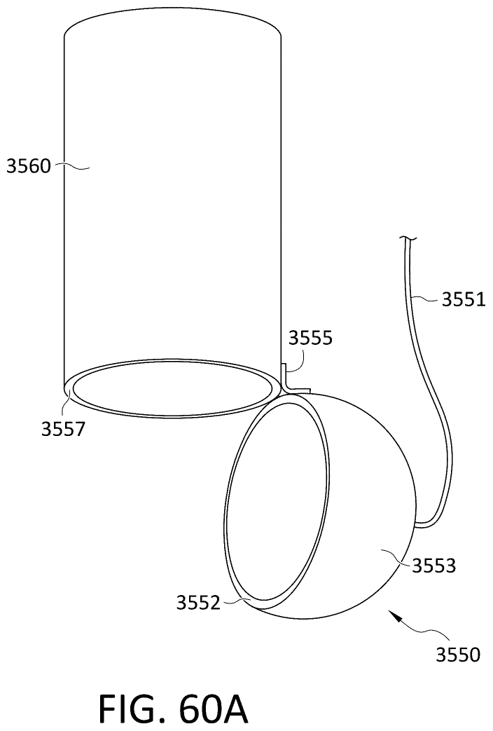

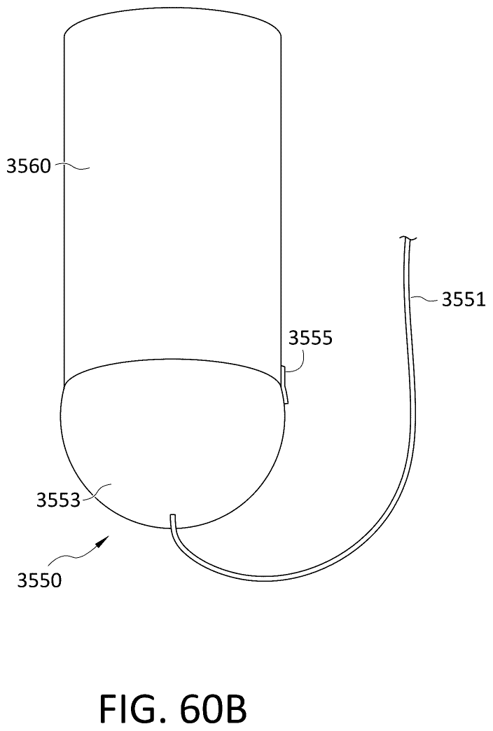

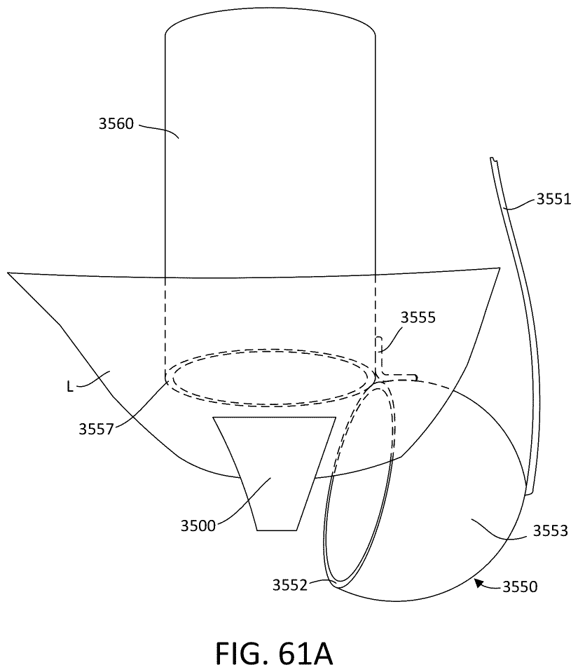

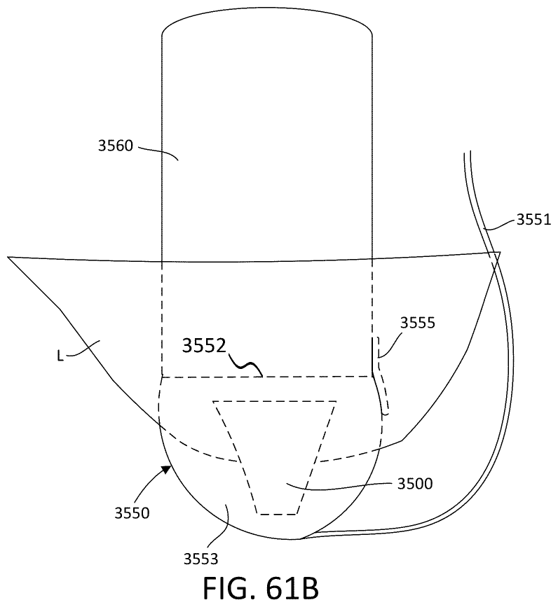

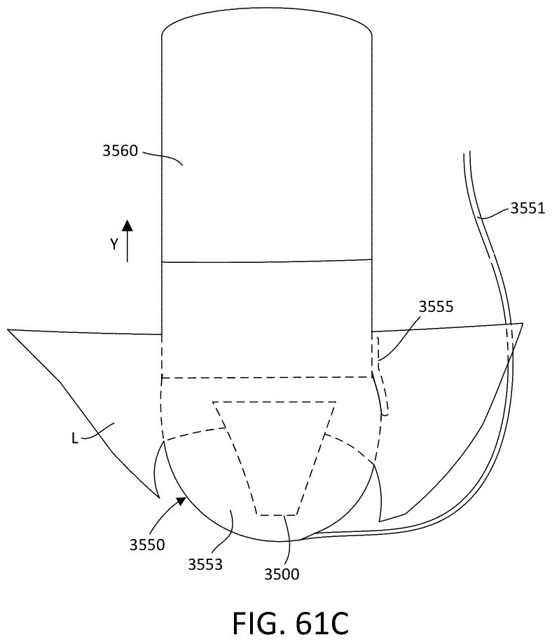

FIGS. 60A-61C illustrate an example system, device, and method for the removal of an implantable device using a retrieval catheter 3560, a retrieval device 3550, and an actuation element 3551. The retrieval device 3550 can be attached to the retrieval catheter 3560 such that the retrieval device 3550 can be moved between an open position (as shown in FIG. 60A) and a closed position (as shown in FIG. 60B) relative to the retrieval catheter 3560 by the actuation element 3551. In implementations, the retrieval device 3550 is attached to the retrieval catheter 3560 by a hinged connection, such as by hinge connector 3555. The actuation element 3551 can be, for example, a suture, a wire, rod, line, and/or any other suitable element that is capable of moving the retrieval device 3550 to open and/or closed positions.

In some implementations, the retrieval device 3550 can be biased in the closed position, and the actuation element 3551 can be used to move the retrieval device to the open position. In some implementations, the retrieval device 3550 can be biased in the open position, and the actuation element 3551 can be used to move the retrieval device to the closed position. In some implementations, the retrieval device 3550 is further attachable to the retrieval catheter 3560 to maintain the retrieval device 3550 in the closed position. That is, the retrieval device 3550 can attach to the retrieval catheter 3560 with a cap-like connection, such as a friction fit connection, a tongue and groove connection, a latch-type connection, a threaded connection, or any other suitable type of connection. The actuation element 3551 can be used to move the retrieval device 3550 from the open position to the closed position and/or between the closed position and the open position.

The retrieval device 3550 can include a cutting element 3552 and a cap 3553. In some implementations, the cap 3553 can be solid material, mesh material, netted material, etc. In some implementations, the cutting element 3552 is configured for cutting, severing and/or ablating one or more leaflets L (e.g., the anterior and/or posterior leaflets of the mitral valve or the anterior, posterior, and/or septal leaflets of the tricuspid valve) of a native heart valve, such as through the use of friction, heat, electrocautery, vibration, blades, serration, cold cutting, or the like. In some implementations, the cutting element 3552 is formed of electrodes that can be comprised of a metallic element allowing current to flow. In some implementations, the cutting element 3552 is connected to an infrared generator, such that the heat can be used to sever the leaflets L of the native heart valve. In some implementations, at least one of the cutting element 3552 and the cap 3553 is made of nitinol or spring wire to allow for shape-memory characteristics. In some implementations, the cutting element 3552 can include surfaces which allow for radiofrequency energy to ablate the tissue around an implantable device. In some implementations, the cutting element 3552 can include cryogenic cutting features that can be used to cut the leaflets L of the native heart valve.

In some implementations, the cutting element 3552 can be disposed on the cap 3553 as illustrated, on or at a distal end 3557 of the retrieval catheter 3560, or both on the cap 3553 and the distal end of the retrieval catheter 3560. When included on both the cap 3553 and the distal end 3557 of the retrieval catheter 3560, the same or different types of cutting mechanisms can be used on the cap 3553 and the distal end 3557 of the catheter 3560. A cutting element 3552 on the distal end 3557 of the retrieval catheter can be configured for cutting, severing, or ablating leaflets L of a native heart valve, such as through the use of friction, heat, electrocautery, vibration, blades, serration, cold cutting, or the like. In some implementations, a cutting element on the distal end 3557 is formed of electrodes that can be comprised of a metallic element allowing current to flow. In some implementations, the cutting element on the distal end 3557 is connected to an infrared generator, such that the heat can be used to sever the leaflets L of the native heart valve. In some implementations, the cutting element on the distal end 3557 can include surfaces which allow for radiofrequency energy to ablate the tissue around an implantable device. In some implementations, the cutting element on the distal end 3557 can include cryogenic cutting features that can be used to cut the leaflets L of the native heart valve.

In some implementations, only one of the distal end 3557 of the retrieval catheter 3560 and the cap 3553 of the retrieval device 3550 have a cutting element 3552 for severing the leaflets L of the native heart valve. In some implementations, both the distal end 3557 of the retrieval catheter 3560 and the cap 3553 of the retrieval device 3550 have cutting elements 3552 for severing leaflets L of the native heart valve.

FIGS. 61A-61C illustrate a method of using the retrieval device 3550 to remove an implantable device 3500 that is secured to one or more leaflets L of a native heart valve. The implantable device 3500 can take any suitable form, such as, for example, any form described in the present application. As shown in FIG. 61A, the retrieval catheter 3560 can be placed through a native heart valve (e.g., by any means described in the present application) such that, when the retrieval device 3550 is in the open position, the implantable device 3500 is positioned between the distal end 3557 of the retrieval catheter 3560 and the retrieval device 3550. When in this position, the implantable device can be below the distal end 3557 of the retrieval catheter 3560 (as shown in the illustrated example), and/or a portion of the implantable device can extend into the retrieval catheter 3560.

Referring to FIG. 61B, subsequently, the retrieval device 3550 is moved to the closed position such that the implantable device 3500 is encapsulated by the retrieval device 3550 and the retrieval catheter 3560. Once the implantable device 3500 is encapsulated by the retrieval device 3550 and the retrieval catheter 3560, the cutting element 3552 on at least one of the retrieval device 3550 and the retrieval catheter 3560 can be used to sever the leaflets L of the native heart valve.

Referring to FIG. 61C, after the leaflet(s) L are severed, the retrieval catheter 3560 is removed from the native heart valve (in the direction Y in the illustrated example) such that the implantable device 3500 and portions of the leaflet(s) L can be removed from the heart of the patient.

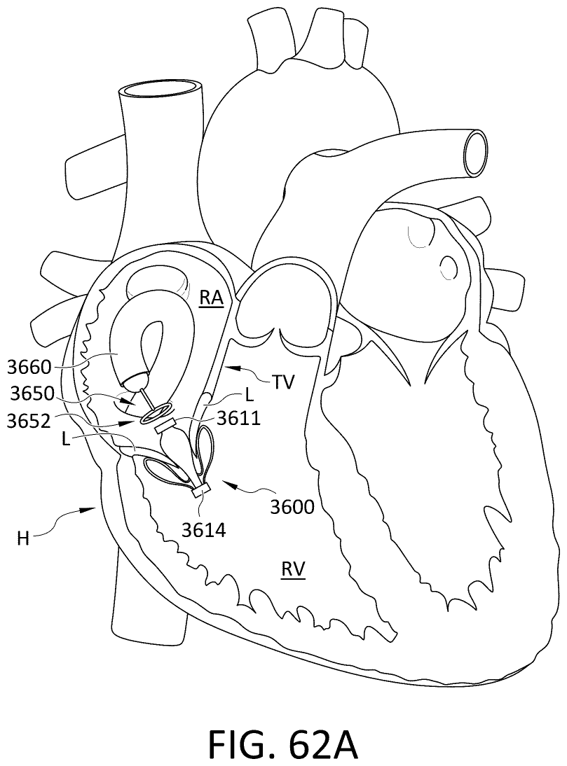

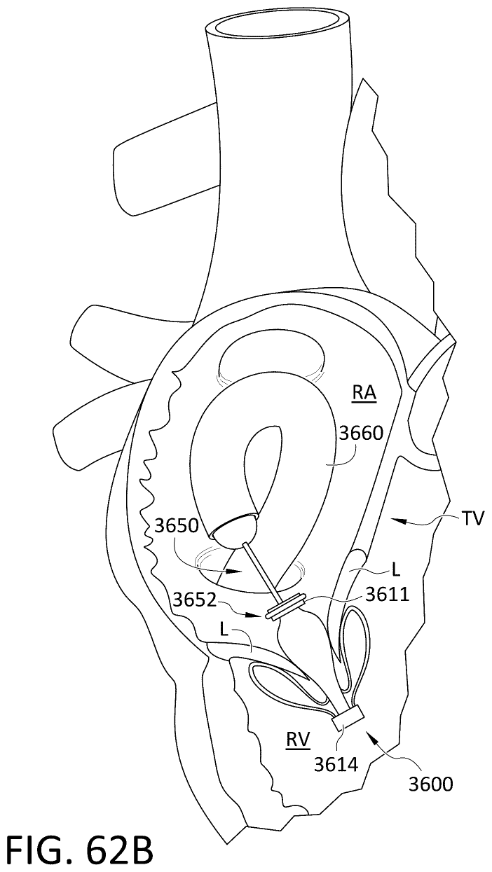

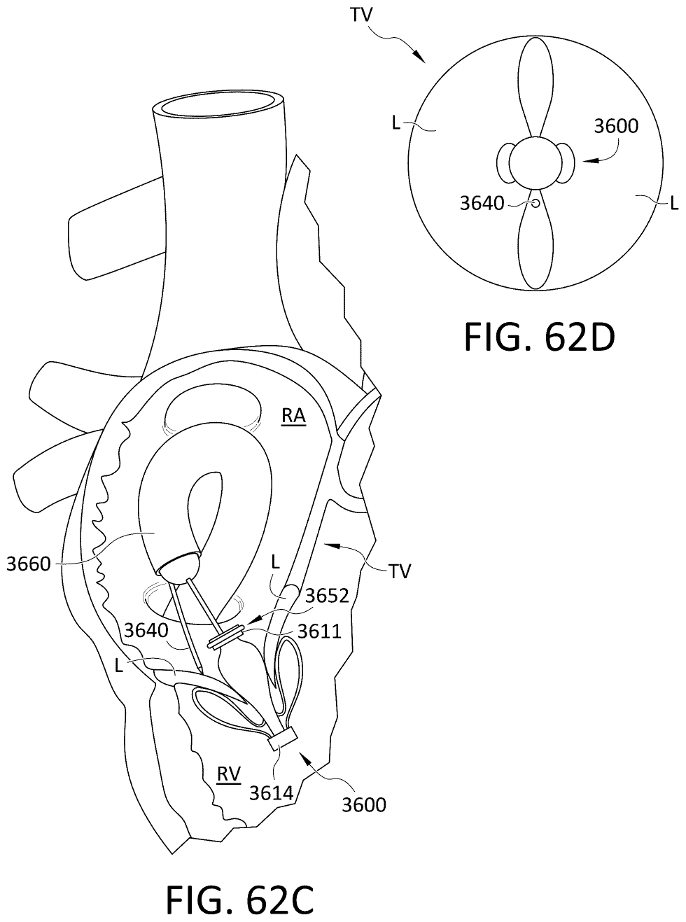

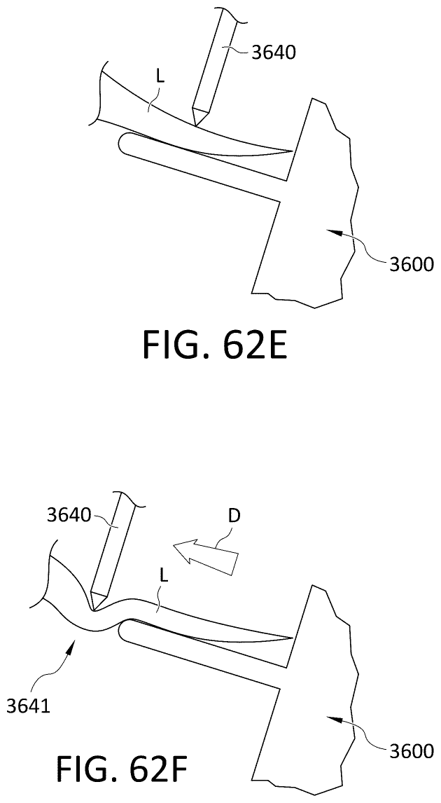

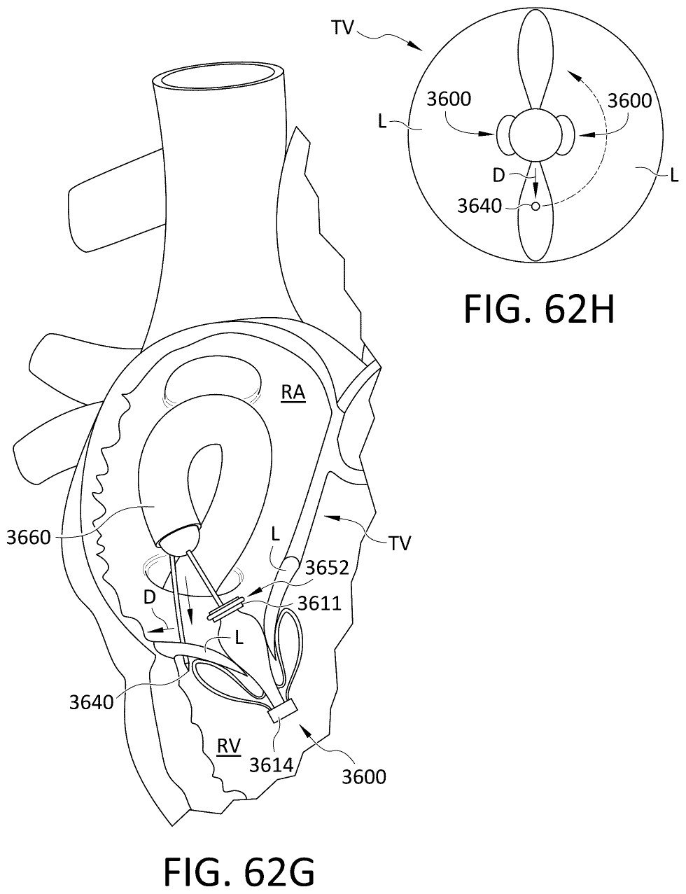

FIGS. 62A-62H illustrate a method, system, and apparatus for removing an implantable device 3600 from at least one leaflet L of a native heart valve using an example cutting device 3640 (See FIG. 62C). The cutting device 3640 can include, for example, a wire, blades, razors, energy-based cutting devices, scissors, any other suitable member for cutting, severing, or ablating one or more leaflets L of a native heart valve, or any combination thereof. The cutting device 3640 can cut the leaflets L, for example, through the use of friction, electrocautery, vibration, serration, cold cutting, or the like. In some implementations, the cutting device 3640 includes an electrosurgical tip or blade formed of electrodes that can be comprised of a metallic element allowing current to flow. In some implementations, the cutting device 3640 is connected to an infrared generator, such that the heat can be used to sever the leaflets L of the native heart valve. In some implementations, the cutting device 3640 can include surfaces which allow for radiofrequency energy to ablate the tissue around an implantable device. In some implementations, at least a portion of the cutting device 3640 can be made of nitinol to allow for shape-memory characteristics. In some implementations, the cutting device 3640 can include cryogenic cutting features that can be used to cut the leaflets L of the native heart valve. The implantable device 3600 can take any suitable form, such as, for example, any form described in the present application.

A catheter 3660 can be used to provide the conduit through which the cutting device 3640 is delivered to the implantable device 3100. In some implementations, the distal end of the retrieval catheter 3660 can also be used to provide a downward force on the proximal side of the leaflet(s) L and/or the implantable device 3600 in order to keep the leaflet(s) taut and/or the implantable device 3600 stable during the resecting process. In some implementations, the inside diameter of the retrieval catheter 3660 can have an inner diameter that allows the implantable device 3600 and portions of the dissected leaflet(s) L to be retracted therethrough.

An optional stabilization component 3650 can be used to secure and hold the implantable device 3600 during cutting of the one or more leaflets L. The stabilization component 3650 can be deployed through the catheter 3660 or a separate catheter (not shown). In some implementations, the stabilization component 3650 has an element for grasping and stabilizing the implantable device 3600, such as a stabilizing snare 3652 (or a lasso, tether, latch, clasp, or other element). The stabilizing snare 3652 can be secured around a portion of the implantable device 3600, such as the collar 3611 (e.g., any collar described in the present application) or the cap 3614 (e.g., any cap described in the present application).

In some implementations, the portion of the implantable device 3600 that the stabilizing snare 3652 attaches to, such as the cap 3614 or collar 3611, can be configured to enhance imaging such that it allows the user to more easily secure the stabilization component 3650 to the implantable device 3600. In some implementations, the stabilization component 3650 can include pincers, graspers, vacuum suction devices, or any other means of docking. In some implementations, the stabilization component 3650 stabilizes the implantable device 3600 such that the cutting device 3640 can more easily be positioned to cut one or more leaflets L of the native heart valve.

In some implementations, when the implantable device is being fully removed from the native heart valve, the stabilization component 3650 can be used to draw the implantable device 3600 into the catheter 3660 such that the implantable device 3600 can be removed from the heart of the patient. In some implementations, a separate retrieval device (e.g., any retrieval device described in the present application) can be used with the catheter 3660 and/or the stabilization component 3650 to remove the implantable device 3600 from the patient's heart.

Referring to FIG. 62A, the catheter 3660 is shown being delivered proximate the tricuspid valve TV of the heart H. The catheter 3660 can be inserted into the right atrium RA by any suitable means, such as, for example, any means described in the present application. Referring to FIG. 62B, the optional stabilization component 3650 is shown being attached to a collar 3611 of the implantable device 3600 to secure and hold the implantable device 3600.

Referring to FIGS. 62C and 62D, the cutting device 3640 is shown being deployed from the catheter 3660 and engaging a leaflet L of the tricuspid valve TV. The cutting device 3640 can be positioned in a coapting region of the tricuspid valve TV. Referring to FIGS. 62E and 62F, in some implementations, the leaflet L can be tented to ensure proper placement of the cutting device 3640 prior to the resecting process. For example, referring to FIG. 62E, the cutting device 3640 is shown engaging the leaflet L, but the leaflet L does not deform or tent due to this engagement because of the implantable device 3600 being positioned on the other side of the leaflet L. Referring to FIG. 62F, in one implementation, moving the cutting device 3640 in an outward direction D allows the engagement between the cutting device 3640 and the leaflet L to cause a deforming or tenting 3641 that indicates the cutting device 3640 is in a proper placement for the resecting process. That is, the implantable device 3600 is no longer in a position relative to the cutting device 3640 that would affect the cutting of the leaflet L by the cutting device 3640. While the illustrated implementation is showing the implantable device preventing or inhibiting tenting 3641 of the leaflet L, it should be understood that other objects can be positioned to prevent or inhibit tenting 3641 and proper cutting of the leaflet L, such as, for example chordae tendinea in the left ventricle LV of the heart H.

Referring to FIGS. 62G and 62H, the cutting device 3640 is shown being moved in the outward direction D such that the cutting device 3640 is positioned away from the implantable device 3600. Referring to FIG. 62H, the cutting device 3640 is shown cutting through a leaflet L in the direction C such that the implantable device 3600 and a cut portion of the leaflet L are removed from one of the leaflets, while the implantable device 3600 remains connected to the other leaflet L. In some implementations, the implantable device 3600 can be removed from two or more leaflets L of the native heart valve.

While the method shown in FIGS. 62A-62H is described with reference to the tricuspid valve TV, it should be understood that this method can also be used for removing the implantable device 3600 from at least one leaflet L of the mitral valve using the example cutting device 3640.

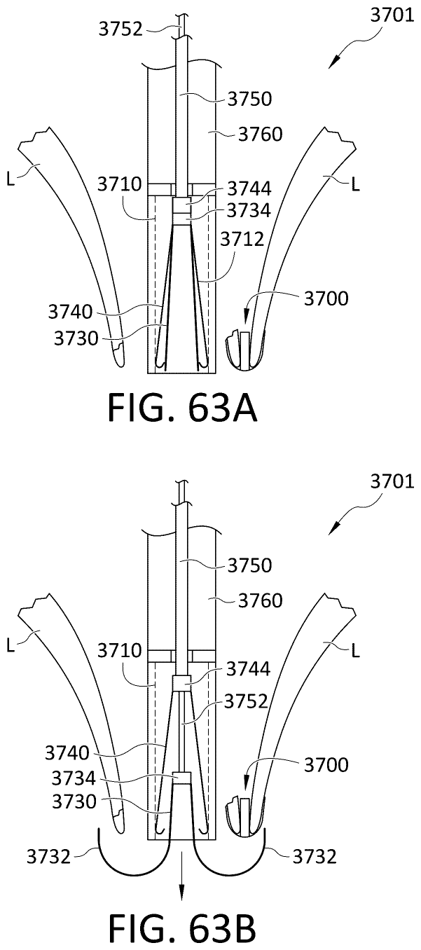

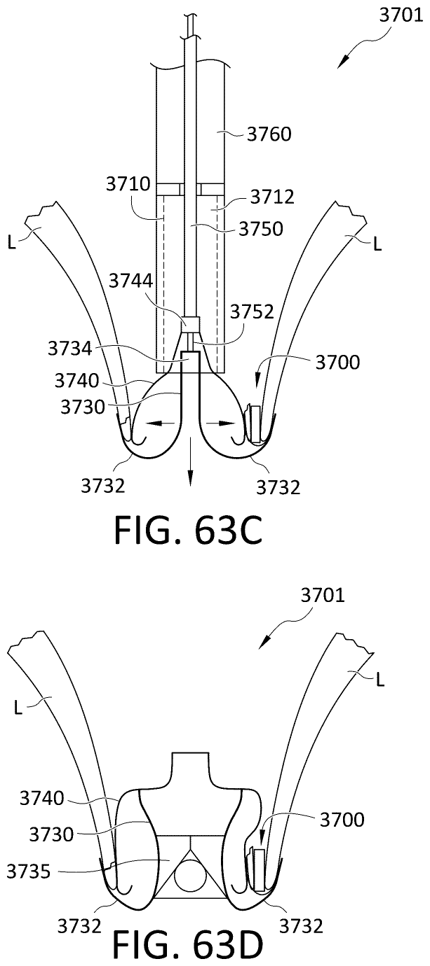

FIGS. 63A-63D illustrate a method of implanting a replacement valve 3701 onto a native valve such that the valve captures an implantable device 3700 that is attached to a native valve. For example, the implantable device 3700 can be attached to at least one leaflet, but not all of the leaflets, of a native heart valve, because the implantable device 3700 was severed from one or more leaflets, such as by using the cutting device 3640 described with reference to FIGS. 62A-62H or any other cutting device or method disclosed in the present application. The implantable device 3700 can take any suitable form, such as, for example, any form described in the present application.

The replacement heart valve 3701 can be configured to be implanted via a delivery system or other means for delivery. The delivery system can comprise one or more of a guide/delivery sheath, a delivery catheter, a steerable catheter, an implant catheter, tube, combinations of these, etc. The replacement heart valve 3701 can be removably coupled to a delivery catheter 3760. The replacement heart valve 3701 can be coupled to the catheter 3760 in a variety of ways, including a releasable coupler, a releasable press fit, friction fit, magnetic fit, a threaded connection, etc. In implementations in which the implantable device is severed from one of the leaflets using the cutting device 3640 and catheter 3660 of FIGS. 62A-62H, the same catheter can optionally be used to deliver both the cutting device 3640 and the replacement heart valve 3701. In some implementations, different catheters can be used to deliver the cutting device 3640 and the replacement heart valve 3701.

The replacement heart valve 3701 can be positioned in a native heart valve between opposing leaflets L. The replacement heart valve 3701 can take any suitable form, such as, for example, any form described in the present application, as well as any form described in U.S. Published Patent Application Pub. Nos. 2020/0368015 and 2020/0297481, and U.S. Pat. Nos. 10,799,938; 10,758,348; and 10,166,097, which are incorporated herein by reference in their entireties. In some implementations, the replacement heart valve can include an inner body 3730, an outer body 3740, and anchors 3732 or the replacement heart valve 3701 can include the body 3740 attached to anchors 3732 and the inner body can be omitted. When included, the inner body 3730 can include a one-way valve 3735 (see FIG. 63D). The optional inner body 3730 can be movable relative to the outer body 3740 or the optional inner body 3730 can be fixed to the outer body 3740. In implementations that do not include the optional inner body 3730, the outer body 3740 can include the one-way valve 3735. The replacement heart valve 3701 can be delivered from an optional capsule 3710 that is attached to a distal end of the catheter 3760 or from the catheter itself.

The inner body or frame 3730 and the outer body or frame 3740 can comprise a variety of shapes and be made from a variety of materials or substances. For example, the inner body or frame 3730 and/or the outer body or frame 3740 can be made from a flexible and/or expandable material. The inner body or frame 3730 and the outer body or frame 3740 can be configured to expand and contract. For example, the inner body or frame 3730 and the outer body or frame 3740 can contract or be compressed to fit within a cavity 3712 of the capsule 3710. In some implementations when the inner body or frame 3730 and/or the outer body or frame 3740 are removed from the cavity 3712 of the capsule 210, the inner body or frame 3730 and the outer body or frame 3740 are configured to expand. For example, the inner body or frame 3730 and/or the outer body or frame 3740 can have a stent or stent-like configuration with struts that allow expansion and contraction.

Referring to FIGS. 63B-63D, the anchors 3732 can take a wide variety of forms, such as, for example, hooks, paddles, gripping elements, or the like. The anchors can be jointed and/or flexible. The anchors 3732 can be curved or rounded such that a leaflet can fit into the curve and be secured between the anchor 3732 and the outer body 3740. In some implementations, the anchors 3732 can include attachment portions or gripping members. The gripping members can comprise clasps, optional barbs, friction-enhancing elements, or other means for securing (e.g., protrusions, ridges, grooves, textured surfaces, adhesive, etc.).

One or more actuation elements can be used during deployment of the replacement heart valve 3701. In the illustrated example, the outer body or frame 3740 is removably coupled to an outer actuation element 3750. In some implementations, the outer body or frame 3740 can be covered by a removable or retractable retaining sleeve. The outer actuation element 3750 can be disposed radially inward of the catheter 3760. The outer actuation element 3750 can be slidable relative to the catheter 3760. In some examples, the outer actuation element 3750 can attach to the outer body or frame 23740 at an outer collar 3744.

The optional inner body or frame 3730 can be removably coupled to an optional inner actuation element 3752 or the inner body or frame 3730 can be fixed to the outer body or frame 3740. When the inner body or frame 3730 is fixed to the outer body or frame 3740, the inner actuation element 3752 can be omitted. The optional inner actuation element 3752 can be disposed radially inward of the outer actuation element 3750. The inner actuation element 3752 can be slidable relative to the outer actuation element 3750. In some examples, the inner actuation element 3752 can attach to the inner body or frame 3730 at an inner collar 3734. The optional inner actuation element 3752, outer actuation element 3750, and the catheter 3760 can all be moved simultaneously and independently of one another.

The outer actuation element 3750 and optional inner actuation element 3752 can take a wide variety of different forms, including a wire, rod, shaft, tube, screw, suture, line, strip, or a combination of these. The outer actuation element 3750 and optional inner actuation element 3752 can be made of a variety of different materials and have a variety of configurations. As one example, the actuation element(s) can be threaded such that rotation of the actuation element moves the valve 3701 or one or more portions of the valve relative to the capsule. Or, the actuation elements can be unthreaded, such that pushing or pulling the actuation element moves the valve 3701 or one or more portions of the valve relative to the capsule.

With reference to FIGS. 63A-63D, the device 3701 can be positioned in a heart valve between opposing leaflets L. With reference to FIG. 63B, the device 3701 can be configured for the inner body or frame 3730 to be moved along the longitudinal axis of the inner actuation element 3752 away from the capsule 3710 in order to create a gap between the capsule 3710 and the anchors 3732. In the illustrated example, the inner body or frame 3730 is pushed out of the outer body or frame 3740 and the capsule 3710. The movement of the inner actuation element 3752 can push the anchors 3732 out of the capsule 3710 and into the ventricular or lower portion of the heart. In some implementations, the optional inner body or frame 3730, the outer body or frame 3740, and anchors 3732 are configured to self-expand as they move distally out from the capsule 3710. For example, the body or frame 3730 can have a self-expanding stent or stent-like configuration.

With reference to FIG. 63C, the device 3701 can be configured for the outer body or frame 3740 to be moved along the longitudinal axis of the outer actuation element 3750 away from the capsule 3710. The movement of the outer actuation element 3750 can push the outer body or frame 3740 out of the capsule 3710 and towards the leaflets L. As a result, the leaflets L can be captured between the outer body or frame 3740 and the anchors 3732. In the illustrated example, the anchors 3732 expand radially outward further than the outer body or frame 3740, such that the anchors 3732 are disposed on an outer or ventricular side of the leaflets L and the outer body or frame 3740 is disposed on an inner or atrial side of the leaflets L. In some implementations, the outer body or frame 3740 is configured to self-expand as it moves distally out from the capsule 3710 or the capsule 3710 is retracted off of the outer body or frame 3740.

Still referring to FIG. 63C, since the implantable device 3700 is attached to one of the leaflets L (e.g., due the resecting process described with reference to FIGS. 62A-62H), the implantable device 3700 is also positioned between the outer body or frame 3740 and the anchors 3732. In some implementations, the capsule 3710 is retracted proximally from the optional inner actuation element 3752 and/or outer actuation element 3750. The outer body or frame 3740 expands radially outward as the outer body or frame extends further out of the capsule 3710 to secure the leaflets L and device 3700 between the outer body or frame 3740 and the anchors.

With reference to FIG. 63D, as the outer body or frame 3740 is completely advanced out of the capsule, the leaflets L and, consequently, the implantable device 3700 are secured against the outer body or frame 3740 and the anchors 3732. In implementations, the expanded outer body or frame 3740 and/or anchors can conform around the device 3700. The actuation element(s) 3750, 3752 and delivery system or catheter 3760 can be decoupled from the device 3701, leaving the device 3701 attached to the native valve leaflets L. Once the device 3701 is implanted on the native valve leaflets L, the implantable device 3700 attached to one or more of the leaflets L is secured in a position against the leaflet L, the anchors 3732 and/or the outer body or frame 3734 such that the device is prevented or inhibited from becoming disconnected. The one-way valve 3735 replaces the function of the native valve. That is, the replacement valve allows normal flow through the native valve and prevents or inhibits retrograde flow through the native valve.

In some implementations configured for use in the tricuspid valve, the replacement heart valve 3701 is configured to be secured to the three tricuspid leaflets such that the one-way valve 3735 of the replacement valve 3701 is positioned between all three native leaflets. In some implementations configured for use in the mitral valve, the replacement heart valve 3701 is configured to be secured to the two mitral leaflets such that the one-way valve of the replacement valve 3701 is positioned between both native leaflets.

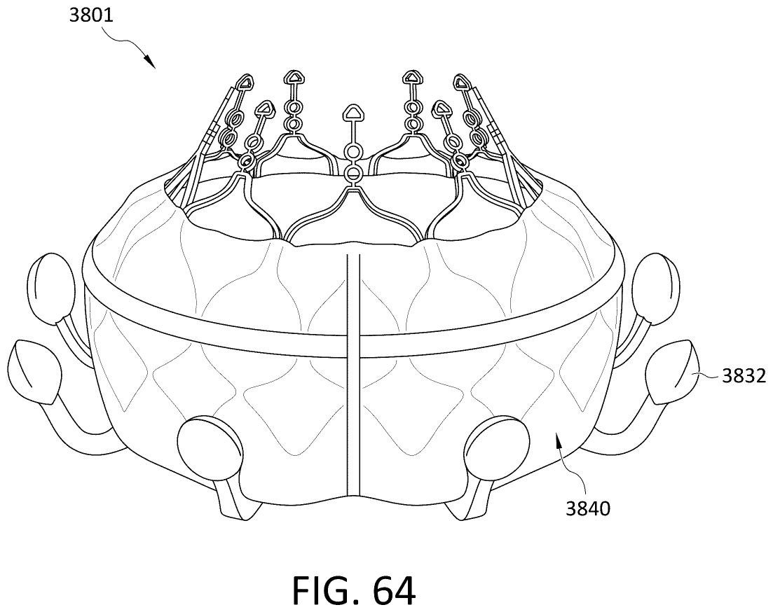

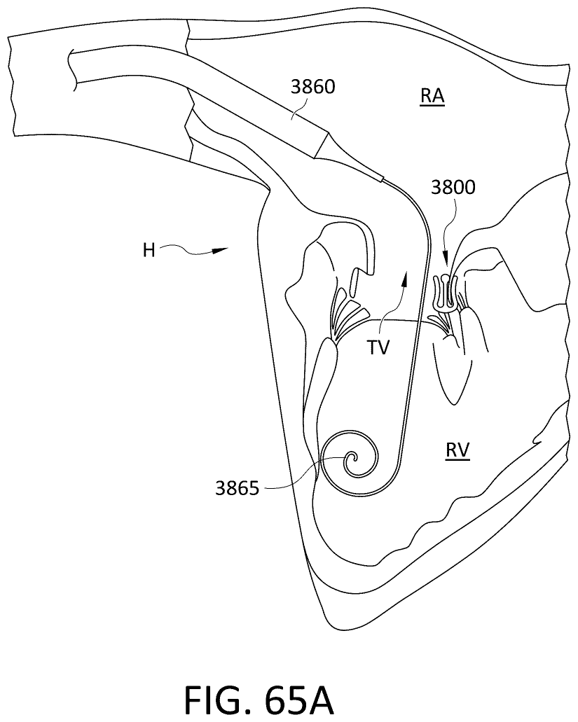

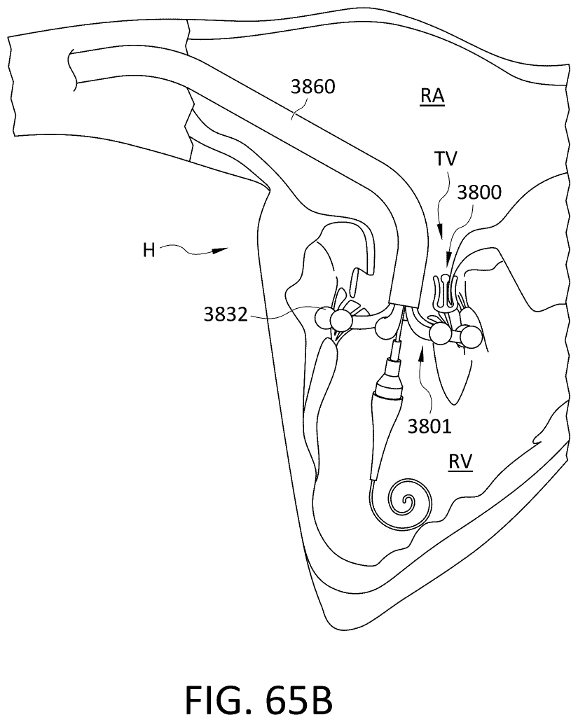

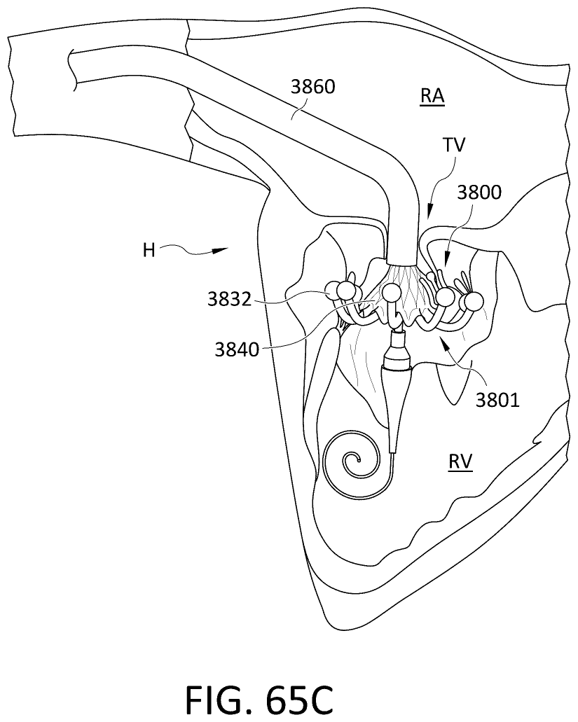

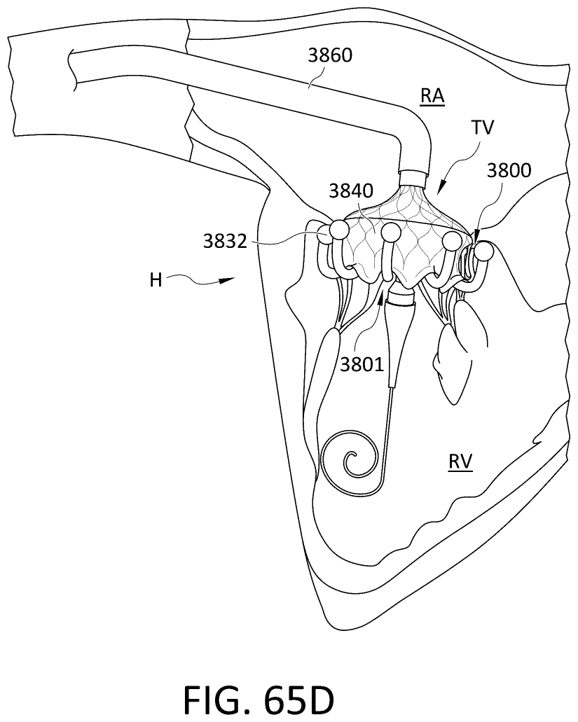

FIG. 64 illustrates an example valve 3801, and FIGS. 65A-65E illustrate a method of implanting the valve 3801 onto a native valve such that the valve 3801 captures an implantable device 3800 that is attached to the native valve. In some implementations, the valve 3801 can optionally be the EVOQUE™ valve from Edwards Lifesciences.

In some implementations, as shown in FIG. 64, the valve 3801 can have anchors 3832 and a body or frame 3840. One or more leaflets of a native heart valve and an implantable device (e.g., implantable device 3800 shown in FIGS. 65A-65E) that is attached to the leaflet(s) can be secured between the anchors 3832 and the body or frame 3840 when the valve is implanted on a native heart valve (e.g., the mitral valve or the tricuspid valve).

Additional details and example designs for a valve, such as the valve 3801 shown in FIG. 64, are described in U.S. Pat. Nos. 8,403,983, 8,414,644, 8,652,203, 10,813,757, and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. The implantable device 3800 can take any suitable form, such as, for example, any form described in the present application.

Referring now to FIGS. 65A-65E, in some implementations, the valve 3801 can be delivered to the native tricuspid valve TV of the heart H such that the valve 3801 can be secured to leaflets L and secure the implantable device 3800 to the leaflet(s) because the implantable device 3800 remains connected to one or more leaflets after a resecting process (e.g., the resecting process shown in FIGS. 62A-62H).

The heart valve 3801 can be configured to be implanted via a delivery system or other means for delivery. The delivery system can comprise one or more of a guidewire, a guide/delivery sheath, a delivery catheter, a steerable catheter, an implant catheter, tube, combinations of these, etc. The heart valve 3801 can be removably coupled to a delivery catheter 3860. The replacement heart valve 3801 can be coupled to the catheter 3860 in a variety of ways, including a releasable coupler, a releasable press fit, friction fit, magnetic fit, a threaded connection, etc. In implementations in which the implantable device is severed using the cutting device 3640 and catheter 3660 of FIG. 62A-62H, the same catheter can optionally be used to deliver both the cutting device 3640 and the replacement heart valve 3801. In some implementations, different catheters can be used to deliver the cutting device 3640 and the replacement heart valve 3801.

Referring to FIG. 65A, an optional guidewire 3865 is inserted through the right atrium RA, through the tricuspid valve TV, and into the right ventricle RV A delivery sheath and/or catheter 3860 is inserted over the optional guidewire 3865 into the right atrium RA. Referring to FIG. 65B, the catheter 3860 is moved into position within the tricuspid valve TV, into the right ventricle RV. The anchors 3832 are partially extended from the catheter 3860 and are thereby partially opened so that the leaflets L can be captured. For example, the delivery catheter 3860 can be advanced and steered or flexed to position the valve 3801 as illustrated by FIG. 65B. An actuation element (not shown) can be advanced from inside the steerable catheter 3860 to engage the valve 3801 and/or the catheter 3860 can be retracted to cause the anchors 3832 of the valve to extend from the catheter 3860. The actuation element can take any suitable form, such as, for example, any form described in the present application.

Referring now to FIG. 65C, the valve 3801 can be further advanced from the catheter 3860 (via the actuation element and/or retraction of the catheter) such that the body or frame 3840 and anchors 3832 are positioned to capture the leaflets L and the attached device 3800. Referring to FIG. 65D, the valve 3801 is fully deployed from the catheter 3860 such that the body or frame 3840 moves to an expanded position that causes an engagement with the anchors 3832. This engagement between the body or frame 3840 and the anchors 3832 captures the leaflets L and, consequently, the implantable device 3800 between the body or frame 3840 and the anchors 3832. In implementations, a portion of the body or frame 3840 and/or some of the anchors 3832 conform to the shape of the device 3800. Referring to FIG. 65E, after the valve is secured to the leaflets L, the catheter 3860 is removed from the heart H with the valve 3801 remaining secured to the leaflets L and capturing the implantable device 3800.

While the method shown in FIGS. 65A-65E is described with reference to the tricuspid valve TV, it should be understood that this method can also be used for securing the valve 3801 to the mitral valve such that the replacement valve captures an implantable device connected to a leaflet of the mitral valve.

Certain conditions or circumstances may necessitate detaching the implantable device from one or more leaflets of the native heart valve. In some implementations, a device and method are provided for detaching the implantable device from a first leaflet while the device remains attached to a second leaflet. In some implementations, a device and method are provided for detaching the implantable device from both a first and second leaflet such that the device can be removed using a retrieval catheter.

In some implementations, the device is configured to resect a native valve leaflet to detach the leaflet from the implantable device. In some implementations, the device is a cutting device. The cutting device can be formed of electrodes that can be comprised of a metallic element allowing current to flow. The cutting device can be made of nitinol. The cutting device can comprise a surface which allows for radiofrequency energy, ultrasonic, or another type of energy to ablate and/or cut the native leaflet.

FIG. 80 illustrates an example implantable device (e.g., the implantable device 3100) deployed between anterior and posterior leaflets 20, 22 of the mitral valve MV. The implantable device 3100 is illustrated having captured both the anterior and posterior leaflet 20, 22 and is positioned generally in the middle of the valve such that a dual orifice valve is formed (i.e., a first or lateral orifice 40 on a first side of the implantable device 3100 and a second or medial orifice 42 on a second side of the implantable device 3100 opposite the first orifice 40).

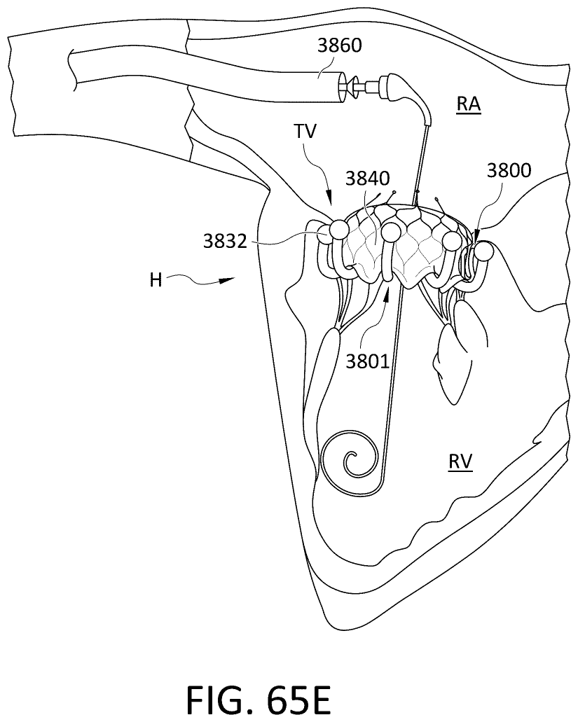

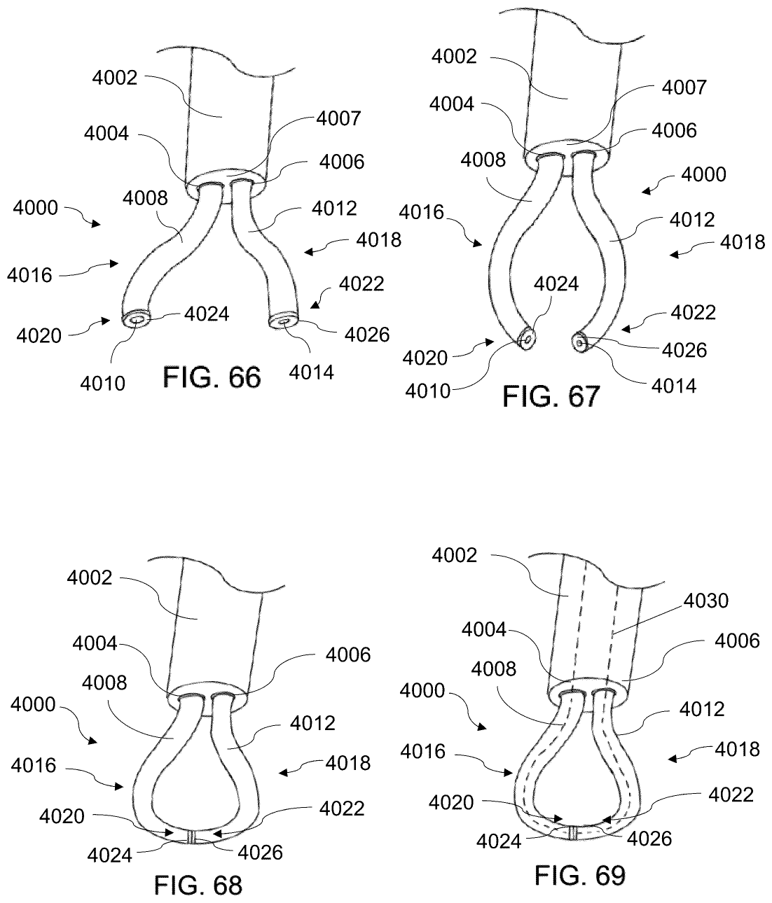

FIGS. 66-72 illustrates an example cutting device 4000 for resecting a native leaflet. The cutting device 4000 can be delivered to the valve by any suitable delivery system, such as any delivery system disclosed herein. The cutting device 4000 can be configured in a variety of ways. In the illustrated implementation, the cutting device 4000 can be delivered via a catheter 4002 (e.g., a steerable catheter or a non-steerable catheter passed through one or more steerable catheters) having a first lumen 4004 and a second lumen 4006 separate from the first lumen 4004 that are shown opening to a distal end 4007 of the catheter 4002.

In the illustrated example, the cutting device 4000 includes a first cutter delivery catheter 4008 having a third lumen 4010 and configured to be delivered through the first lumen 4004 and a second cutter delivery catheter 4012 having a fourth lumen 4014 and configured to be delivered through the second lumen 4006. The first cutter delivery catheter 4008 includes a bendable and/or flexible first distal end portion 4016 and the second cutter delivery catheter 4012 includes a bendable and/or flexible second distal end portion 4018. In some implementations, the first distal end portion 4016 and/or the second distal end portion 4018 are steerable. The first distal end portion 4016 and/or the second distal end portion 4018 can be steerable by any suitable means. In some implementations, the first distal end portion 4016 and/or the second distal end portion 4018 include a shape memory alloy, such as for example, nitinol, to allow for shape-memory characteristics. Thus, the first distal end portion 4016 and/or the second distal end portion 4018 can be shape set to a desired position. For example, the first distal end portion 4016 and the second distal end portion 4018 can be shape set to the positions illustrated by FIGS. 68 and 69 such that the first distal end portion 4016 and the second distal end portion 4018 do not need to be steerable.

The first cutter delivery catheter 4008 includes a first distal tip 4020 and the second cutter delivery catheter 4012 includes a second distal tip 4022. In some implementations, the first distal tip 4020 is configured to releasably couple to the second distal tip 4022 such that the third lumen 4010 aligns with (e.g., is operatively connected to) the fourth lumen 4014. In some implementations, the first distal tip 4020 can include a first coupling element 4024 and the second distal tip 4022 can include a second coupling element 4026. The first coupling element 4024 and the second coupling element 4026 can be configured in a variety of ways, such as male/female connectors, magnets, hook and loop fasteners, detents, a threaded coupling, clasps, grippers, or other suitable couplers.

In the illustrated implementation, the first coupling element 4024 is a first annular magnet and the second coupling element 4026 is a second annular magnet. The first coupling element 4024 and the second coupling element 4026 are arranged on the first distal tip 4020 and the second distal tip 4022, respectively, to attract each other (i.e., unlike poles facing distally) such that the first distal tip 4020 and the second distal tip 4022 couple to each other when in proximity to each other.

Referring to FIG. 66, the first cutter delivery catheter 4008 is illustrated extending from the first lumen 4004 of the catheter 4002. The first cutter delivery catheter 4008 can extend from the first lumen 4004 through the first or lateral orifice 40 to the ventricular side of the valve and the second cutter delivery catheter 4012 can extend from the second lumen 4006 through the second or medial orifice 42 to the ventricular side of the valve (See FIG. 80). In some implementations, instead of both the first cutter delivery catheter 4008 and the second cutter delivery catheter 4012 extending to the ventricular side of the valve, one of the first cutter delivery catheter 4008 or the second cutter delivery catheter 4012 can remain on the atrial side of the valve and the other of the first cutter delivery catheter 4008 or the second cutter delivery catheter 4012 can extend from the atrial side to the ventricular side and back to the atrial side. For example, the first cutter delivery catheter 4008 can extend from the first lumen 4004 through the first or lateral orifice 40 to the ventricular side of the valve and through the second or medial orifice 42 to the atrial side of the valve to connect to the second cutter delivery catheter 4012.

Referring to FIGS. 67-68, once the first distal end portion 4016 of the first cutter delivery catheter 4008 and the second distal end portion 4018 of the second cutter delivery catheter 4012 are on the ventricular side of the valve, the first distal tip 4020 and the second distal tip 4022 couple to each other such that the third lumen 4010 and the fourth lumen 4014 are aligned or operatively connected. In some implementations, the first distal end portion 4016 and the second distal end portion 4018 are steerable and thus can be steered to bring the first distal tip 4020 and the second distal tip 4022 into proximity with each other such that the first coupling element 4024 and the second coupling element 4026 couple together. In some implementations, the first distal end portion 4016 and the second distal end portion 4018 are shape-set such that when extended from the catheter 4002 bend into a position that places the first distal tip 4020 and the second distal tip 4022 into proximity with each other such that the first coupling element 4024 and the second coupling element 4026 couple together. In some implementations, the flexibility of the first distal end portion 4016 and the second distal end portion 4018, without being shape set or steerable, results in the first distal tip 4020 and the second distal tip 4022 being movable into proximity with each other such that the first coupling element 4024 and the second coupling element 4026 couple together.

Referring to FIG. 69, once the first distal tip 4020 and the second distal tip 4022 are connected, a cutting element 4030 for cutting, severing, or ablating the anterior leaflet 20 and/or the posterior leaflet 22 can be advanced through the third lumen 4010 and back through the fourth lumen 4014 or vice versa. The cutting element 4030 can be configured to cut, sever, or ablate the leaflets in a variety of ways, such as through the use of friction, heat, electrocautery, vibration, blades, serration, or the like. (Illustrated in dashed line in FIG. 69). In the illustrated implementation, the cutting element 4030 is a conductive wire (e.g., made of metal or, having a metallic element, allowing current to flow).

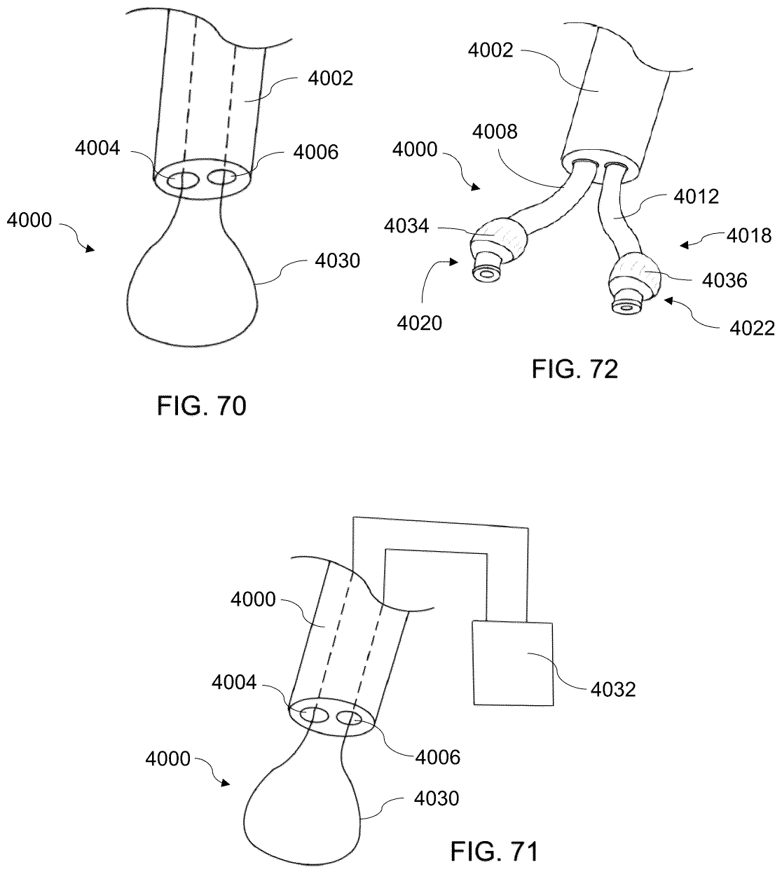

Referring to FIG. 70, once the cutting element is advanced through the third lumen 4010 and back through the fourth lumen 4014, or vice versa, the first cutter delivery catheter 4008 and the second cutter delivery catheter 4012 can be withdrawn, over the cutting element 4030, through the first lumen 4004 and the second lumen 4006 of the catheter 4002, respectively. The first coupling element 4024 and the second coupling element 4026 can be decoupled by pulling the first cutter delivery catheter 4008 and the second cutter delivery catheter 4012 apart (e.g., applying tension to one, or both, of the first cutter delivery catheter 4008 and the second cutter delivery catheter 4012).

Referring to FIG. 71, before or after the first cutter delivery catheter 4008 and the second cutter delivery catheter 4012 have been withdrawn through the catheter 4002, the cutting element 4030 of the illustrated implementation, can be connected to an activation source 4032 configured to energize, heat up, vibrate, or otherwise activate the cutting element 4030. In the illustrative implementation, the cutting element 4030, which is configured as a conductive wire, the activation source 4032 is a radiofrequency generator connected to the cutting element 4030 to supply radiofrequency energy through the cutting element 4030. The radiofrequency energy activates (i.e., heats up) the cutting element 4030. The activated cutting element 4030 can then be pulled through the portion of the anterior leaflet 20, or the posterior leaflet 22, adjacent to where the implantable device 3100 grasps the anterior leaflet 20, or the posterior leaflet 22. As a result, the cut and/or ablated anterior leaflet 20, or cut and/or ablated posterior leaflet 22, is detached from the implantable device 3100.

Referring to FIG. 72, in some implementations, the first cutter delivery catheter 4008 includes a first inflatable balloon 4034 at, or adjacent, the first distal tip 4020 and the second cutter delivery catheter 4012 includes a second inflatable balloon 4036 at, or adjacent, the second distal tip 4022. The first inflatable balloon 4034 can be inflated when the first distal tip 4020 extends through the first or lateral orifice 40 and past the chordae tendineae CT (e.g., FIG. 3). When inflated, the first inflatable balloon 4034 can displace the chordae tendineae CT away from the implantable device 3100 such that the first distal tip 4020 does not extend between individual chordae tendineae CT or minimizes the number of individual chordae tendineae CT between the first distal end portion 4016 of the first cutter delivery catheter 4008 and the implantable device 3100. In doing so, few or no chordae tendineae CT are severed when the cutting element 4030 detaches the leaflet 20, 22 from the implantable device 3100. Similarly, when inflated, the second inflatable balloon 4036 can displace the chordae tendineae CT away from the implantable device 3100 so that the second distal tip 4022 does not extend between individual chordae tendineae CT or minimizes the number of individual chordae tendineae CT between the second distal end portion 4018 of the second cutter delivery catheter 4012 and the implantable device 3100.

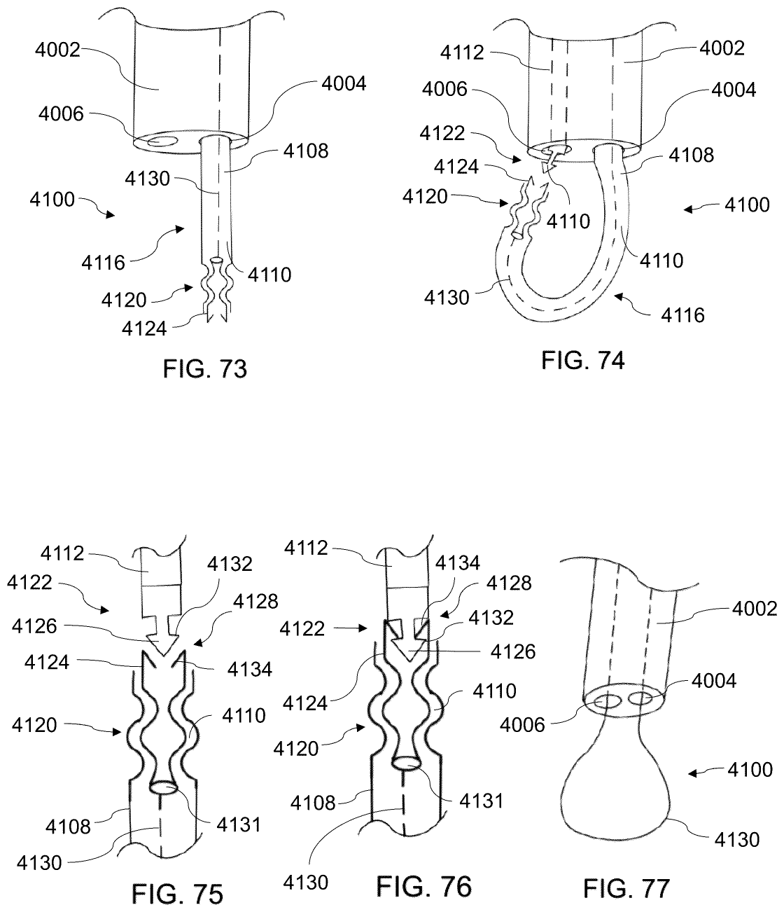

FIGS. 73-77 illustrates an example cutting device 4100 for resecting a native leaflet. The cutting device 4100 can be delivered to the valve by any suitable delivery system, such as any delivery system disclosed herein. The cutting device 4100 can be configured in a variety of ways. In the illustrated implementation, the cutting device 4100 can be delivered via the catheter 4002 having the first lumen 4004 and the second lumen 4006.

In the illustrated example, the cutting device 4100 includes a first cutter delivery catheter 4108 having a third lumen 4110 and configured to be delivered through the first lumen 4004 and a second cutter delivery element 4112 (e.g., catheter, pusher, shaft, rod, etc.) configured to be delivered through the second lumen 4006. The first cutter delivery catheter 4108 includes a bendable and/or flexible first distal end portion 4116. In some implementations, the first distal end portion 4116 is steerable. The first distal end portion 4116 can be steerable by any suitable means. In some implementations, the first distal end portion 4116 can include a shape memory alloy, such as for example, nitinol, to allow for shape-memory characteristics. Thus, the first distal end portion 4116 can be shape set to a desired position. In some implementations, the second cutter delivery element 4112 can be configured similar to the first cutter delivery catheter 4108. For example, the second cutter delivery element 4112 can have a bendable or flexible portion, or a steerable portion, or a shape-set portion.

The first cutter delivery catheter 4108 includes a first distal tip 4120 and the second cutter delivery element 4112 includes a second distal tip 4122. In some implementations, the first distal tip 4120 is configured to couple to the second distal tip 4122. In some implementations, the first distal tip 4120 can include a first coupling element 4124 and the second distal tip 4122 can include a second coupling element 4126. The first coupling element 4124 and the second coupling element 4126 can be configured in a variety of ways, such as male/female connectors, magnets, hook and loop fasteners, detents, a threaded coupling, clasps, grippers, or other suitable couplers.

In the illustrated implementation, the first coupling element 4124 is a female connector releasably coupled to the first cutter delivery catheter 4108. The first coupling element 4124 can be releasably coupled to the first cutter delivery catheter 4108 in a variety of ways. In the illustrative implementation, the first coupling element 4124 is received within the third lumen 4110 at, or adjacent, the first distal tip 4120. In some implementations, the first coupling element 4124 is received within the third lumen 4110 via a friction fit and/or detent and is releasable from the third lumen 4110 with application of sufficient axial force to separate the first coupling element 4124 from the first cutter delivery catheter 4108. In some implementations, an exterior surface of the first coupling element 4124 and an inner surface of the third lumen 4110 can have complementary shapes that act as a detent. Any suitable complementary shapes can be used. In the illustrated example, the first coupling element 4124 and an inner surface of the third lumen 4110 has a complementary shape that is wavy or hourglass shaped.

Referring to FIGS. 75-76, the first coupling element 4124 includes a distal end 4128 and a proximal end 4131 opposite the distal end 4128 (FIGS. 75-76). In the illustrated implementation, the second coupling element 4126 is a male connector fixed the second cutter delivery element 4112 to the form the second distal tip 4122. The second coupling element 4126 is configured to be received within the distal end 4128 of the first coupling element 4124 and the distal end 4128 of the first coupling element 4124 is configured to retain the second coupling element 4126 within the distal end 4128 once received. The second coupling element 4126 and the distal end 4128 of the first coupling element 4124 can be configured in a variety of ways. In the illustrated implementation, the second coupling element 4126 includes one or more radially outward extending projections 4132 (e.g., barbs) and the distal end 4128 includes one or more radially inward extending projections 4134.

In the illustrated example, the cutting device 4100 includes a cutting element 4130 for cutting, severing, or ablating the anterior leaflet 20 and/or the posterior leaflet 22. The cutting element 4130 can be configured to cut, sever, or ablate the leaflets in a variety of ways, such as using friction, heat, electrocautery, vibration, blades, serration, or the like (illustrated in dashed line in FIGS. 73 and 74). In the illustrated implementation, the cutting element 4130 is a conductive wire (e.g., made of metal or, having a metallic element, allowing current to flow) having an end attached to the first coupling element 4124.

Referring to FIGS. 73-74, the first cutter delivery catheter 4108 is illustrated extending from the first lumen 4004 of the catheter 4002. The first cutter delivery catheter 4108 can extend from the first lumen 4004 through one of the first orifice 40 or second orifice 42 (see FIG. 80) to the ventricular side of the heart valve (e.g., mitral valve MV). Once the first distal end portion 4116 of the first cutter delivery catheter 4108 extends through one of the first orifice 40 or second orifice 42, the first distal end portion 4116 can bend (e.g., be steered or shape-set) around the implanted device 3100 and back through the other of the first orifice 40 or second orifice 42. FIG. 74 illustrates the second cutter delivery element 4112 extending through the second lumen 4006 such that the second coupling element 4126 extends out of the second lumen 4006 to connect to the first coupling element 4124.

Referring to FIGS. 75-76, to connect the first coupling element 4124 and the second coupling element 4126, the second coupling element 4126 is pressed into the distal end 4128 of the first coupling element 4124, or the distal end 4128 of the first coupling element 4124 is pressed over the second coupling element 4126. When the second coupling element 4126 extends into the distal end 4128 the radially inward extending projections 4134 are displaced (e.g., flexed or bent) to allow the radially outward extending projections 4132 to pass by. Once the radially outward extending projections 4132 have moved axially past the radially inward extending projections 4134, the radially inward extending projections 4134 flex back to an original position, as shown in FIG. 76, to capture the second coupling element 4126 and connect the first cutter delivery catheter 4108 to the second cutter delivery element 4112.

Once the first coupling element 4124 and the second coupling element 4126 are connected, the first coupling element 4124 can be detached from the first cutter delivery catheter 4108. For example, sufficient tension can be applied to the first cutter delivery catheter 4108 (e.g., pulled back through the first lumen 4004) to pull the first distal tip 4120 over the first coupling element 4124, which is held in place by the second cutter delivery element 4112 via the connection between the first coupling element 4124 and the second coupling element 4126. Once the first cutter delivery catheter 4108 is decoupled from the first coupling element 4124, the first cutter delivery catheter 4108 can be withdrawn through the first lumen 4004 exposing the cutting element 4130.

Further, once the first cutter delivery catheter 4108 is decoupled from the first coupling element 4124, the second cutter delivery element 4112 can be pulled back through the second lumen 4006 which pulls the first coupling element 4124 and cutting element 4130 with it. As a result, the cutting element 4130 can be looped through the first and second lumen 4004, 4006, as shown in FIG. 77. In a manner like the cutting device 4100 illustrated in FIG. 71, the cutting element 4130 can be connected to an activation source (e.g., activation source 4032) configured to energize, heat up, vibrate, or otherwise activate the cutting element 4130. In the illustrative implementation, the cutting element 4130, which is configured as a conductive wire, the activation source is a radiofrequency generator connected to the cutting element 4130 to supply radiofrequency energy through the cutting element 4130. The radiofrequency energy activates (i.e., heats up) the cutting element 4130.

The activated cutting element 4130 can then be pulled through the portion of the anterior leaflet 20, or the posterior leaflet 22, adjacent to where the implantable device 3100 grasps the anterior leaflet 20, or the posterior leaflet 22. As a result, the cut and/or ablated anterior leaflet 20, or cut and/or ablated posterior leaflet 22, is detached from the implantable device 3100.

In some implementations, the second cutter delivery element 4112 can include a conductive element or be a conductive element and the first coupling element 4124 and the second coupling element 4126 can electrically couple the cutting element 4130 to the conductive element. In some implementations, the first coupling element 4124 and the second coupling element 4126 can be electrically conductive such that when coupled together form an electrical connection between the second cutter delivery element and the cutting element 4130. Therefore, in some implementations, the first coupling element 4124 does not need to be pulled back through the second lumen 4006 by second cutter delivery element 4112 in order to be connected to the activation source 4032 since an electrical circuit is formed when the first coupling element 4124 and the second coupling element 4126 are joined.

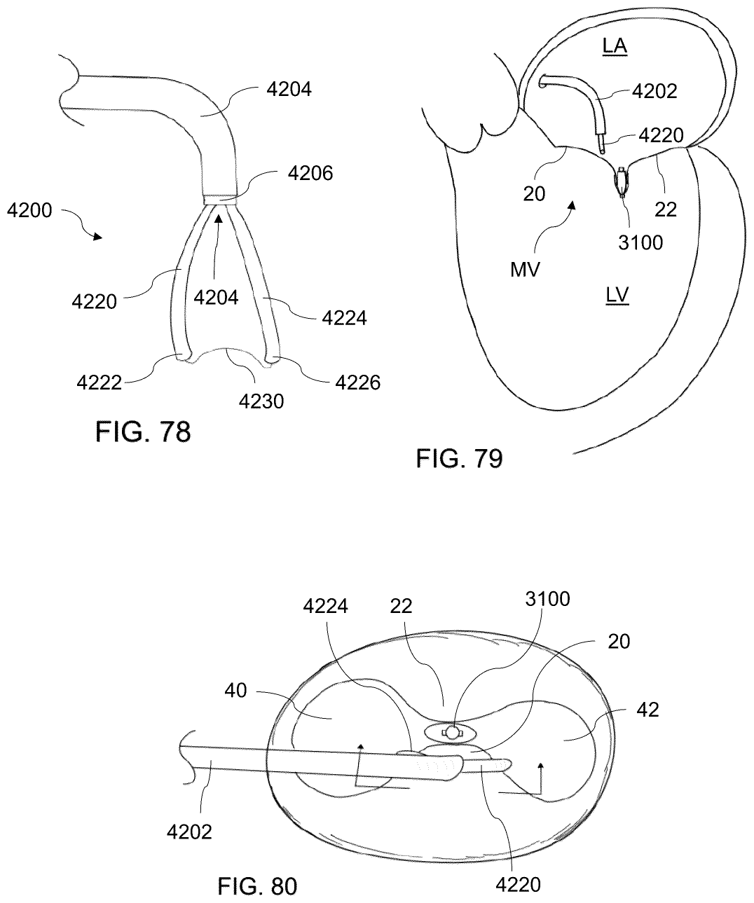

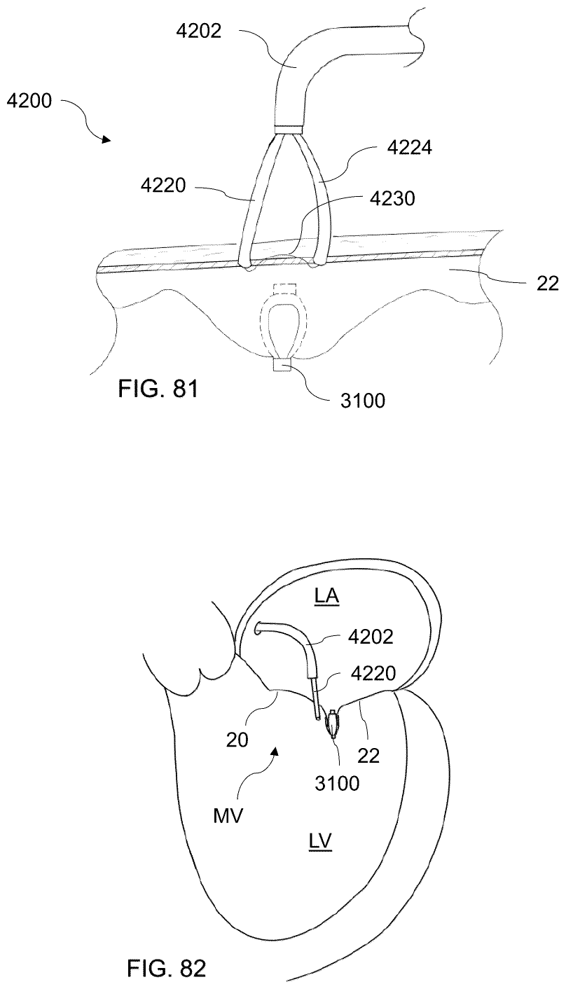

FIGS. 78-84 illustrates an example cutting device 4200 for resecting a native leaflet. The cutting device 4200 can be delivered to the valve by any suitable delivery system, such as any delivery system disclosed herein. The cutting device 4200 can be configured in a variety of ways. In the illustrated implementation, the cutting device 4200 can be delivered via a catheter 4202 having one or more lumens 4204 and a distal tip 4206.

In the illustrated example, the cutting device 4200 is formed as a fork or V-shape having a first arm or prong 4220 with a first distal end 4222 and second arm or prong 4224 with a second distal end 4226 that is spaced apart from the first distal end 4222. Extending between the first distal end 4222 and the second distal end 4226 is a cutting element 4230 for cutting, severing, or ablating the anterior leaflet 20 and/or the posterior leaflet 22. The cutting element 4230 can be configured to cut, sever, or ablate the leaflets in a variety of ways, such as using friction, heat, electrocautery, vibration, blades, serration, or the like. In the illustrated implementation, the cutting element 4230 is a conductive wire (e.g., made of metal or, having a metallic element, allowing current to flow). In some implementations, the cutting element 4230 extending between the first distal end 4222 and the second distal end 4226 is in a slack condition (i.e., not taut).

The first arm 4220 and the second arm 4224 can be configured in a variety of ways. For example, the first arm 4220 and the second arm 4224 can be tubes or catheters through which the cutting element 4230 extends. In some implementations, the first arm 4220 and the second arm 4224 can include a shape memory alloy (e.g., nitinol) or spring wire to allow for shape-memory characteristics. In some implementations, the first arm 4220 and the second arm 4224 can be insulated wire while the cutting element is formed from the same wire with the insulation removed.

Referring to FIGS. 79-81, the cutting device 4200 can be delivered to the left atrium LA via the catheter 4202. Once extended out of the catheter 4202, the first arm 4220 and second arm 4224 spread apart, as shown in FIG. 78. The cutting device 4200 can be positioned over one of the leaflets 20, 22 at a location adjacent the installed implantable device 3100 such that the cutting element 4230 extends across the atrial side of the leaflet 20, 22 (illustrated as the anterior leaflet 20 of the mitral valve MV in FIG. 80).

As shown in FIG. 84, the cutting element 4230 can be connected to an activation source (e.g., activation source 4032) configured to energize, heat up, vibrate, or otherwise activate the cutting element 4230. In the illustrative implementation, for the cutting element 4230, which is configured as a conductive wire, the activation source is a radiofrequency generator connected to the cutting element 4230 to supply radiofrequency energy through the cutting element 4230. The radiofrequency energy activates (i.e., heats up) the cutting element 4230.

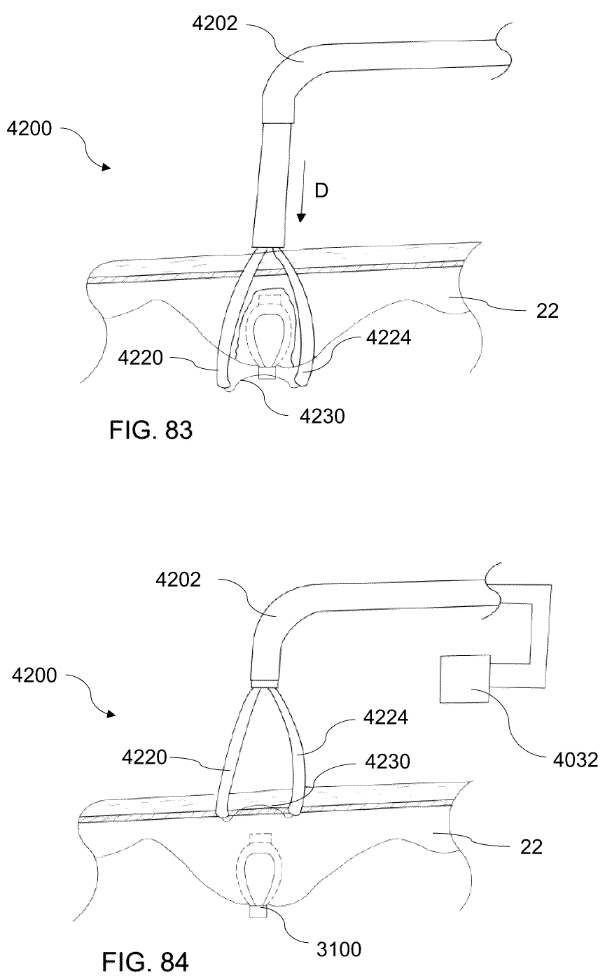

Referring to FIGS. 82-83, once the cutting element 4320 is positioned above the atrial side of the leaflet 20, 22 adjacent the installed implantable device 3100, the activated cutting element 4230 can then be moved downward, toward the left ventricle LV, through the portion of the leaflet (e.g., the anterior leaflet 20 in FIGS. 82-83) adjacent to where the implantable device 3100 grasps the anterior leaflet 20. As a result, the cut and/or ablated anterior leaflet 20 is detached from the implantable device 3100.

While many of the implementations herein are in the context of removing an implant, the systems, apparatuses, devices, methods, etc. herein can be adapted to cut or remove native tissue, even if no implant is present.

Examples (some non-limiting examples are disclosed below):

Example 1. A device for resecting a native leaflet comprising:
a catheter;
a cutting device disposed within the catheter, wherein the cutting device comprises a snare capable of severing or ablating the native leaflet; and
a stabilization component comprising an element for grasping an implantable device.

Example 2. The device of Example 1, wherein the cutting device is formed of electrodes that can be comprised of a metallic element allowing a current to flow.

Example 3. The device of Examples 1 or 2, wherein the cutting device is made of nitinol.

Example 4. The device of any one of Examples 1-3, wherein the cutting device comprises a surface which allows for radiofrequency energy to ablate the native leaflet.

Example 5. The device of any one of Examples 1-4, wherein the stabilization component is configured to attach to a collar of the implantable device.

Example 6. The device of any one of Examples 1-5, wherein the element for grasping the implantable device is a snare.

Example 7. The device of any one of Examples 1-5, wherein the element for grasping the implantable device is any one of a pincer, a grasper, or a vacuum suction device.

Example 8. The device of any one of Examples 1-7, further comprising a second cutting device comprising a second snare capable of severing or ablating the native leaflet.

Example 9. The device of Example 8, wherein the second cutting device is formed of electrodes that can be comprised of a metallic element allowing a current to flow.

Example 10. The device of any one of Examples 8-9, wherein the second cutting device comprises a surface that allows for radiofrequency energy to ablate the native leaflet.

Example 11. The device of any one of Examples 8-10, wherein the second cutting device serves as the stabilization component.

Example 12. A device for resecting a native leaflet comprising:
a catheter;
a cutting device comprising at least one coring element disposed proximate to the catheter, wherein the at least one coring element comprises a feature capable of severing or ablating the native leaflet; and
a stabilization component comprising an element for grasping an implantable device.

Example 13. The device of Example 12, wherein the at least one coring element is disposed along an external surface of the catheter.

Example 14. The device of Example 13, wherein the device comprises a single coring element surrounding the external surface of the catheter.

Example 15. The device of any one of Examples 12-13, wherein the device comprises a first coring element and a second coring element.

Example 16. The device of any one of Examples 12-15, wherein the at least one coring element is arc shaped.

Example 17. The device of any one of Examples 12-16, wherein the feature capable of severing or ablating the native leaflet is a blade.

Example 18. The device of any one of Examples 12-16, wherein the feature capable of severing or ablating the native leaflet is a cutting tip formed of electrodes that can be comprised of a metallic element allowing current to flow.

Example 19. The device of any one of Examples 12-18, wherein the at least one coring element is made of nitinol.

Example 20. The device of any one of Examples 12-16, wherein the feature capable of severing or ablating the native leaflet is a cutting tip formed of electrodes that can be comprised of a metallic element allowing current to flow.

Example 21. The device of any one of Examples 12-18, wherein the at least one coring element further comprises a surface which conducts radiofrequency energy.

Example 22. The device of any one of Examples 12-21, wherein the element for grasping the implantable device is a snare.

Examples 23. The device of any one of Examples 12-21, wherein the element for grasping the implantable device is any one of a pincer, a grasper, or a vacuum suction device.

Example 24. A device for resecting a native leaflet comprising:
a catheter;
a cutting device comprising at least one electrosurgical element;
a stabilization component comprising an element for grasping an implantable device; and
an indicator or gauge.

Example 25. The device of Example 24, wherein the cutting device is made of nitinol.

Example 26. The device of any one of Examples 24-25, wherein the at least one electrosurgical element is a cutting tip or blade.

Example 27. The device of any one of Examples 24-25, wherein the at least one electrosurgical element is an electrosurgical ring.

Example 28. The device of any one of Examples 24-27, wherein the element for grasping the implantable device is a snare.

Example 29. The device of any one of Examples 24-27, wherein the element for grasping the implantable device is any one of a pincer, a grasper, or a vacuum suction device.

Example 30. The device of any one of Examples 25-29, further comprising a second cutting device comprising a second cutting tip.

Example 31. The device of any one of Examples 25-30, wherein the indicator or gauge is a radiopaque feature.

Example 32. The device of any one of Examples 25-31, wherein the indicator or gauge is a depth gauge.

Example 33. The device of any one of Examples 25-31, wherein the indicator or gauge is a long, compliant positioning wire or rod.

Examples 34. The device of any one of Examples 25-31, wherein the indicator or gauge is configured to lead the cutting device and engage tissue to be cut before the cutting device.

Example 35. A method of resecting a native leaflet comprising:
deploying a catheter to an implantable device secured to at least one leaflet of a native heart valve;
deploying a cutting device disposed within the catheter to the at least one leaflet;
securing at least one of the cutting device and a stabilization component to a portion of the implantable device;
resecting the native leaflet with the cutting device;
resecting a second native leaflet with the cutting device; and
removing the implantable device from the native heart valve using at least one of the cutting device and the stabilization component via the catheter.

Example 36. A device for resecting an implantable device from a native leaflet comprising:
a catheter;
a clamp comprising a first grasping arm and a second grasping arm disposed within the catheter, and
wherein the clamp further comprises an element capable of cutting, severing, or resecting the native leaflet.

Example 37. The device of Example 36, wherein upon closure of the first grasping arm and the second grasping arm of the clamp, the first grasping arm and the second grasping arm surround the implantable device.

Example 38. The device of any of Examples 36-37, wherein the first grasping arm and the second grasping arm each further comprise serrated edges or blades.

Example 39. The device of any of Examples 36-38, wherein the first grasping arm and the second grasping arm each further comprise an electrocautery element.

Example 40. A device for resecting a native leaflet comprising:
a catheter;
a cutting device comprising a central wire, a first prong, and a second prong, wherein the cutting device is capable of cutting, severing, or ablating the native leaflet; and
a stabilization component comprising an element configured to grasp an implantable device.

Example 41. The device of Example 40, wherein the first prong and the second prong comprise sharp blades.

Example 42. The device of Example 40, wherein the first prong and the second prong comprise an electrocautery element.

Example 43. The device of Example 40, wherein the cutting device is connected to an infrared generator, such that heat can be used to sever the native leaflet.

Example 44. The device of Example 40, wherein the first prong and the second prong comprise a surface that allows radiofrequency energy to ablate the native leaflet.

Example 45. The device of any of Examples 40-44, wherein the cutting device is made of nitinol.

Example 46. The device of any of Examples 40-45, further comprising a balloon coupled to the cutting device.

Example 47. The device of Example 46, wherein the balloon is configured to cut, sever, or ablate the native leaflet.

Example 48. A device for resecting a native leaflet comprising:
a first catheter;
a first cutting device comprising a hook disposed within the first catheter;
a second catheter;
a second cutting device comprising a loop disposed within the second catheter; and
a stabilization component comprising an element for grasping an implantable device.

Example 49. The device of Example 48, wherein the hook and the loop are capable of cutting, severing, or ablating the native leaflet, such as through the use of friction, heat, electrocautery, vibration, blades, or serration.

Example 50. The device of any of Examples 48-49, wherein the hook and the loop are formed of electrodes that can be comprised of a metallic element allowing current to flow.

Example 51. The device of any of Examples 48-50, wherein the hook and the loop are connected to an infrared generator, such that heat is generated to sever the native leaflet.

Example 52. The device of any of Examples 48-51, wherein the hook and the loop are made of nitinol or spring wire.

Example 53. The device of any of Examples 48-52, wherein the hook and the loop are comprised of surfaces which allow for radiofrequency energy to ablate the native leaflet.

Example 54. The device of any of Examples 48-53, wherein the hook aligns perpendicularly to the loop when it deploys from the second catheter, and wherein the hook is aligned to enter the loop creating a lasso that can be used to sever the native leaflet.

Example 55. A device for resecting a native leaflet comprising:
a catheter;
a retrieval device comprising a positioning element, a snare, and/or a bag, wherein the retrieval device is capable of cutting, severing, or ablating the native leaflet such as through the use of friction, heat, electrocautery, vibration, blades, serration; and
wherein the retrieval device is configured to capture a valve repair device.

Example 56. The device of Example 55, wherein the snare is comprised of an electrocautery element.

Example 57. The device of Example 55, wherein the snare is connected to an infrared generator, such that the heat can be used to sever the native leaflet.

Example 58. The device of any of Examples 55-57, wherein at least one of the snare and the bag is made of nitinol or spring wire to allow for shape-memory characteristics.

Example 59. The device of Example 55 or 58, wherein the snare is comprised of surfaces which allow for radiofrequency energy to ablate the native leaflet.

Example 60. A device for resecting a native leaflet comprising:
a catheter having a distal end;
a cap attached to the catheter such that the cap is movable between an open position and a closed position relative to the catheter;
one or more cutting elements connected to one or more of the distal end of the catheter and the cap;
an actuation element configured to move the cap between the open position and the closed position;
wherein the one or more cutting elements is configured to cut, sever, or ablate the native leaflet; and
wherein the cap is configured to capture a valve repair device.

Example 61. The device of Example 60, wherein the one or more cutting elements is configured to cut, sever, or ablate the native leaflet by the use of at least one of friction, heat, electrocautery, vibration, blades, and serration.

Example 62. The device of Example 60 or 61, wherein the one or more cutting elements comprise a single cutting element on the cap.

Example 63. The device of Example 62, wherein the cutting element is comprised of an electrocautery element.

Example 64. The device of Example 62, wherein the cutting element is connected to an infrared generator, such that the heat can be used to sever the native leaflet.

Example 65. The device of Example 62, wherein the cutting element comprises surfaces that allow for radiofrequency energy to ablate the native leaflet.

Example 66. The device of any of Examples 60-65, wherein the one or more cutting elements comprise a single cutting element disposed on the distal end of the catheter.

Example 67. The device of Example 66, wherein the cutting element is comprised of an electrocautery element.

Example 68. The device of Example 66, wherein the cutting element is connected to an infrared generator, such that the heat can be used to sever the native leaflet.

Example 69. The device of Example 66, wherein the cutting element comprises one or more surfaces configured to apply radiofrequency energy to ablate the native leaflet.

Example 70. The device of any of Examples 60-69, wherein at least one of the cutting element and the cap is made of nitinol or spring wire to provide shape-memory characteristics.

Example 71. The device of any of Examples 60-70, wherein the cap is connected to the catheter by a hinge connector.

Example 72. The device of any of Examples 60-71, wherein the cap is biased in the closed position, and wherein the actuation element is used to move the cap from the closed position to the open position.

Example 73. The device of any of Examples 60-72, wherein the actuation element comprises a wire.

Example 74. A method for resecting one or more native leaflets of a native valve and implanting a replacement valve to the native valve comprising:
deploying a cutting device such that the cutting device is positioned proximate the native valve;
cutting the one or more native leaflets with the cutting device such that a valve repair device is removed from the one or more native leaflets, wherein the valve repair device remains connected to at least one other native leaflet of the native valve;
deploying the replacement valve such that the replacement valve is positioned proximate the native valve, the replacement valve having a body and one or more anchors; and
attaching the replacement valve to at least a portion of the one or more native leaflets and the at least one other native leaflet of the native valve such that the valve repair device is captured by the replacement valve between the body and the one or more anchors.

Example 75. The method of Example 74, further comprising engaging the one or more native leaflets with the cutting device prior to cutting the one or more native leaflets and repositioning the cutting device relative to the one or more native leaflets if tenting of the one or more native leaflets is not detected.

Example 76. The method of any of Examples 74-75, wherein the cutting comprises the use of at least one of friction, electrocautery, vibration, and serration.

Example 77. The method of any of Examples 74-76, wherein the cutting device comprises one or more blades.

Example 78. The method of any of Examples 74-76, wherein the cutting device comprises an electrosurgical tip formed of electrodes that includes a metallic element configured to allow current to flow.

Example 79. The method of any of Examples 74-76, wherein the cutting device comprises an electrosurgical tip formed of electrodes that includes a metallic element configured to allow current to flow.

Example 80. The method of any of Examples 74-76, wherein the cutting device is connected to an infrared generator such that heat can be used to sever the one or more native leaflets.

Example 81. The method of any of Examples 74-76, wherein the cutting device comprises one or more surfaces that allow for radiofrequency energy to ablate the one or more native leaflets.

Example 82. The method of any of Examples 74-81, further comprising positioning a delivery system proximate the native valve, the delivery system being configured to deploy the cutting device and the replacement valve.

Example 83. The method of Example 82, further comprising deploying a stabilization component to connect the valve repair device to the delivery system prior to cutting the one or more native leaflets.

Example 84. The method of any of Examples 82-83, wherein the cutting device is deployed from a first catheter of the delivery system and the replacement valve is deployed from a second catheter of the delivery system.

Example 85. The method of any of Examples 74-84, wherein the body comprises an inner body and an outer body.

Example 86. A system for resecting a native leaflet, comprising:
a catheter having a first lumen and a second lumen;
a first cutter delivery catheter configured to be delivered through the first lumen, the first cutter delivery catheter having a third lumen and a first distal tip;
a second cutter delivery catheter configured to be delivered through the second lumen, the second cutter delivery catheter having a second distal tip; and
a cutting element configured to extend through the third lumen;
wherein the first distal tip includes a first coupling element configured to connect to a second coupling element on the second distal tip such that the cutting element is advanceable through the second lumen.

Example 87. The system of Example 86, wherein the first coupling element is configured to magnetically couple to the second coupling element.

Example 88. The system of Example 86 or 87, wherein the second cutter delivery catheter includes a fourth lumen, and wherein when the first distal tip is connected to the second distal tip, the third lumen is aligned with the fourth lumen.

Example 89. The system of Example 88, wherein the cutting element is advanceable through the second lumen via the fourth lumen.

Example 90 The system of Example 89, wherein the first coupling element is a first annular magnet, and the second coupling element is a second annular magnet.

Example 91. The system of Example 86, wherein the first coupling element is configured to mechanically couple to the second coupling element.

Example 92. The system of Example 91, wherein the first coupling element is configured as a female connector and the second coupling element is configured as a male connector.

Example 93. The system of Example 92, wherein the first coupling element is configured to be received in the third lumen at the first distal tip.

Example 94. The system of Example 93, wherein the first coupling element has a first exterior surface having a complementary shape to an interior surface of the first distal tip.

Example 95. The system of any of Examples 92-94, wherein the first coupling element includes a distal end and a proximal end, wherein the cutting element is attached to the proximal end.

Example 96. The system of Example 95, wherein the distal end is configured to receive the second coupling element.

Example 97. The system of any of Examples 92-96, wherein the second coupling element includes one or more radially outward extending projections.

Example 98. The system of Example 97, wherein the first coupling element includes one or more radially inward extending projections configured to engage the one or more radially outward extending projections to resist separation of the second coupling element from the first coupling element.

Example 99. The system of any of Examples 92-98, wherein the first coupling element is configured to be removed from the first cutter delivery catheter once coupled to the second coupling element.

Example 100. The system of any of Examples 86-99, wherein the cutting element is a conductive wire.

Example 101. The system of Example 100, further comprising an activation source configured to energize the cutting element.

Example 102. The system of Example 101, wherein the activation source is a radio-frequency generator.

Example 103. The system of any of Examples 86-102, wherein the first cutter delivery catheter has a distal end portion that is steerable.

Example 104. The system of any of Examples 86-102, wherein the first cutter delivery catheter has a distal end portion that has shape-memory characteristics.

Example 105. The system of any of Examples 86-104, further comprising an inflatable balloon attached to an exterior surface of the first cutter delivery catheter adjacent the first distal tip.

Example 106. A system for resecting a native leaflet, comprising:
a delivery catheter;
a cutting device configured for delivery through the delivery catheter, the cutting device comprising:

a first arm having a first distal end;
a second arm having a second distal end spaced apart from the first distal end; and
a cutting element extending between the first distal end and the second distal end.

Example 107. The system of Example 106, wherein the cutting element is a conductive wire.

Example 108. The system of Example 107, wherein the conductive wire is in a slack condition between the first distal end and the second distal end.

Example 109. The system of any of Examples 106-108, wherein the first arm and the second arm form a V-shape.

Example 110. The system of any of Examples 106-108, wherein the first arm and the second arm are insulated conductive wire and the cutting element is uninsulated conductive wire.

Example 111. The system of Example 106, further comprising an activation source configured to energize the cutting element.

Example 112. The system of Example 111, wherein the activation source is a radio-frequency generator.

Example 113. A method of resecting a native valve leaflet captured by an implantable device, the method comprising:
extending a first cutter delivery catheter from an atrial side of the native valve leaflet to a ventricular side on a first side of the implantable device,
extending a second cutter delivery catheter from the atrial side of the native valve leaflet to the ventricular side on a second side of the implantable device opposite the first side;
connecting the first cutter delivery catheter to the second cutter delivery catheter on the ventricular side of the native valve leaflet;
extending a cutting element through a first lumen in the first cutter delivery catheter and a second lumen in the second cutter delivery catheter;
withdrawing the first cutter delivery catheter to expose the cutting element adjacent the native valve leaflet; and
moving cutting element through the native valve leaflet from the ventricular side to the atrial side to detach the native valve leaflet from the implantable device.

Example 114. The method of Example 113, wherein connecting the first cutter delivery catheter to the second cutter delivery catheter further comprises magnetically coupling the first cutter delivery catheter to the second cutter delivery catheter.

Example 115. The method of Example 114, wherein connecting the first cutter delivery catheter to the second cutter delivery catheter further comprises aligning the first lumen in the first cutter delivery catheter with the second lumen in the second cutter delivery catheter.

Example 116. The method of any of Examples 113-115 further comprising steering a first distal tip of the first cutter delivery catheter toward a second distal tip of the second cutter delivery catheter in the ventricular side.

Example 117. The method of any of Examples 113-116, further comprising connecting the cutting element to an activation source.

Example 118. The method of Example 117, wherein the activation source is a radio frequency generator.

Example 119. The method of any of Examples 113-118, further comprising activating the cutting element with radiofrequency energy.

Example 120. The method of any of Examples 113-119, wherein the cutting element is a conductive wire.

Example 121. The method of any of Examples 113-120, wherein extending the first cutter delivery catheter from the atrial side of the native valve leaflet to the ventricular side further comprises inflating a balloon on an exterior surface of the first cutter delivery catheter.

Example 122. A method of resecting a native valve leaflet captured by an implantable device, the method comprising:
extending a first cutter delivery catheter from an atrial side of the native valve leaflet to a ventricular side on a first side of the implantable device, wherein a cutting element is connected to a first coupling element associated with the first cutter delivery catheter;
extending a second cutter delivery catheter from the atrial side of the native valve leaflet to the ventricular side on a second side of the implantable device opposite the first side;
connecting the first cutter delivery catheter to the second cutter delivery catheter on the ventricular side of the native valve leaflet;
withdrawing the first cutter delivery catheter to expose the cutting element adjacent the native valve leaflet; and
moving cutting element through the native valve leaflet from the ventricular side to the atrial side to detach the native valve leaflet from the implantable device.

Example 123. The method of Example 122, wherein connecting the first cutter delivery catheter to the second cutter delivery catheter further comprises mechanically coupling the first cutter delivery catheter to the second cutter delivery catheter.

Example 124. The method of Example 123, wherein mechanically coupling the first cutter delivery catheter to the second cutter delivery catheter further comprises receiving a male connector into a female connector.

Example 125. The method of Example 124, wherein withdrawing the first cutter delivery catheter further comprises detaching the female connector from the first cutter delivery catheter.

Example 126. The method of Example 125, wherein detaching the female connector from the first cutter delivery catheter further comprises applying a tensile force to one or both of the first cutter delivery catheter and the second cutter delivery catheter.

Example 127. The method of any of Examples 122-126, further comprising connecting the cutting element to an activation source.

Example 128. The method of Example 127, wherein the activation source is a radio frequency generator.

Example 129. The method of any of Examples 122-128, further comprising activating the cutting element with radiofrequency energy.

Example 130. The method of any of Examples 122-129, wherein the cutting element is a conductive wire.

Example 131. The method of any of Examples 122-130, wherein extending the first cutter delivery catheter from the atrial side of the native valve leaflet to the ventricular side further comprises inflating a balloon on an exterior surface of the first cutter delivery catheter.

Example 132. The method of any of Examples 122-131, wherein connecting the first cutter delivery catheter to the second cutter delivery catheter on the ventricular side of the native valve leaflet further comprises steering a distal end portion of the first cutter delivery catheter toward the second cutter delivery catheter.

Example 133. A method of resecting a native valve leaflet captured by an implantable device, the method comprising:
delivering a distal tip of a delivery catheter to an atrial side of the native valve leaflet;
supporting a cutting element adjacent the atrial side of the native valve leaflet; and
moving cutting element through the native valve leaflet from the atrial side to a ventricular side to detach the native valve leaflet from the implantable device.

Example 134. The method of Example 133, wherein the cutting element is supported adjacent the atrial side of the native valve leaflet in a slack condition.

Example 135. The method of Example 133 or 134, further comprising connecting the cutting element to an activation source.

Example 136. The method of Example 135, wherein the activation source is a radio frequency generator.

Example 137. The method of any of Examples 133-136, further comprising activating the cutting element with radiofrequency energy.

Example 138. The method of any of Examples 133-137, wherein the cutting element is a conductive wire.

Example 139. The method of any of Examples 133-138, wherein supporting the cutting element adjacent the atrial side of the native valve leaflet further comprises extending the cutting element between a first distal end of a first arm and a second distal end of a second arm.

Example 140. The method of Example 139, further comprising extending the first arm and the second arm from the distal tip of the delivery catheter.

Any of the various systems, assemblies, devices, apparatuses, etc. in this disclosure (including in the above examples) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A method for resecting one or more native leaflets of a native valve and implanting a replacement valve in the native valve comprising:
deploying a cutting device such that the cutting device is positioned proximate the native valve;
cutting the one or more native leaflets with the cutting device such that a valve repair device is removed from the one or more native leaflets, wherein the valve repair device remains connected to at least one other native leaflet of the native valve;
deploying the replacement valve such that the replacement valve is positioned proximate the native valve, the replacement valve having a body and one or more anchors; and
attaching the replacement valve to at least a portion of the one or more native leaflets and the at least one other native leaflet of the native valve such that the valve repair device is captured by the replacement valve between the body and the one or more anchors.

2. The method of claim 1, further comprising engaging the one or more native leaflets with the cutting device prior to cutting the one or more native leaflets and repositioning the cutting device relative to the one or more native leaflets if tenting of the one or more native leaflets is not detected.

3. The method of claim 1, wherein the cutting device comprises a first cutter delivery catheter and a second cutter delivery catheter and the method further comprises magnetically connecting the first cutter delivery catheter to the second cutter delivery catheter.

4. The method of claim 1, wherein the cutting device comprising a central wire, a first prong, and a second prong and wherein the cutting device is capable of cutting, severing, or ablating the native leaflet.

5. The method of claim 4, wherein the first prong and the second prong each comprise an electrocautery element.

6. The method of claim 1, wherein the cutting device comprises at least one coring element disposed proximate to a catheter, wherein the at least one coring element comprises a feature capable of severing or ablating the native leaflet.

7. The method of claim 1, wherein the cutting device comprises a first snare capable of severing or ablating the native leaflet and a second snare capable of severing or ablating the native leaflet.

8. The method of claim 1, further comprising leading the cutting device with a gauge or indicator and engaging tissue to be cut with the gauge or indicator before the cutting device.

9. The method of claim 1, wherein the cutting comprises the use of at least one of friction, electrocautery, vibration, and serration.

10. The method of claim 1, wherein the cutting device comprises an electrosurgical tip formed of electrodes that includes a metallic element configured to allow current to flow.

11. The method of claim 1, wherein the cutting device is connected to an infrared generator such that heat can be used to sever the one or more native leaflets.

12. The method of claim 1, further comprising positioning a delivery system proximate the native valve, the delivery system being configured to deploy the cutting device and the replacement valve.

13. The method of claim 1, further comprising deploying a stabilization component to connect the valve repair device to a delivery system of the replacement valve prior to cutting the one or more native leaflets.

14. A device for resecting a native leaflet comprising:
a catheter;
a retrieval device capable of cutting, severing, or ablating the native leaflet such as through the use of friction, heat, electrocautery, vibration, blades, serration; and
wherein the retrieval device is configured to capture a valve repair device.

15. The device of claim 14, wherein the retrieval device comprises a positioning element, a snare, and a bag, wherein the bag is configured to capture the valve repair device.

16. The device of claim 14, wherein the retrieval device comprises a cap attached to the catheter such that the cap is movable between an open position and a closed position relative to the catheter, an actuation element configured to move the cap between the open position and the closed position, and wherein the cap is configured to capture the valve repair device.

17. The device of claim 14, wherein the retrieval device comprises a clamp comprising a first grasping arm and a second grasping arm disposed within the catheter.

18. The device of claim 17, wherein upon closure of the first grasping arm and the second grasping arm of the clamp, the first grasping arm and the second grasping arm surround the valve repair device.

19. The device of claim 18, wherein the first grasping arm and the second grasping arm each further comprise serrated edges or blades.

20. A method of implanting a replacement valve to a native valve comprising:
deploying the replacement valve to a native valve leaflet where a valve repair device is connected to the native valve leaflet;
wherein the replacement valve has a body and one or more anchors; and
attaching the replacement valve to the native valve leaflet such that the valve repair device is captured by the replacement valve between the body and the one or more anchors.
